(19) United States
(12) Patent Application Publication
(10) Pub. No.: US 2010/0192254 A1
(43) Pub. Date: Jul. 29, 2010

(54) METHOD OF INCREASING RESISTANCE AGAINST SOYBEAN RUST IN TRANSGENIC PLANTS

(75) Inventors: Markus Frank, Neustadt (DE); Holger Schultheiss, Neustadt (DE); Caroline Höfle, Wolfersdorf (DE)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 12/376,795

(22) PCT Filed: Aug. 9, 2007
(86) PCT No.: PCT/EP07/58268

§ 371 Date: Feb. 9, 2009

(30) Foreign Application Priority Data
Aug. 10, 2006 (EP) 06118753.0

(51) Int. Cl. A01H 1/00; C12N 15/82; A01H 5/00; C12N 5/10

(52) U.S. Cl. 800/279; 435/468; 800/301; 435/419

(57) ABSTRACT

The present invention relates to a method of increasing resistance against soybean rust in transgenic plants and/or plant cells, as well as to the use of a nucleic acid molecule for the production of these plants and/or plant cells. In these plants, the content and/or the activity of at least one MLO protein is altered in comparison to the wild-type plants. Furthermore, the invention relates to transgenic plants and/or plant cells having an increased resistance against soybean rust and to expression vectors comprising a sequence that is identical, homologous or complementary to a sequence encoding an functional or non-functional MLO or fragments thereof.

### BACKGROUND OF THE INVENTION

The present invention relates to a method of increasing resistance against soybean rust in transgenic plants and/or plant cells, as well as to the use of a nucleic acid molecule for the production of these plants and/or plant cells. In these plants, the content and/or the activity of at least one MLO protein is altered in comparison to the wild-type plants. Furthermore, the invention relates to transgenic plants and/or plant cells having an increased resistance against soybean rust and to expression vectors comprising a sequence that is identical, homologous or complementary to a sequence encoding an functional or non-functional MLO or fragments thereof.

Plants are permanently confronted with pathogenic microbes. Plant diseases caused by various pathogens, such as viruses, bacteria and fungi, can lead to substantial crop losses in the growing of cultivated plants, with economic consequences on the one hand, but also pose a threat for the safety of human food on the other hand. Chemical fungicides have been used since the last century to control fungi diseases. Although the use of these agents led to a reduction in the extent of plant diseases, up to now it cannot be ruled out that these compounds may have harmful effects on humans, animals and the environment. In order to reduce the use of traditional pesticides to a minimum, it is therefore important to examine the natural pathogen defense of various plants to different pathogens, and to make—in addition to the classical breeding methods—systematic use of genetic engineering, such as by introducing external resistance genes, or by manipulating endogenous gene expression in plants for the production of pathogen resistant plants.

Resistance generally means the ability of a plant to prevent, or at least curtail the infestation and colonization by a harmful pathogen. Different mechanisms can be discerned in the naturally occurring resistance, with which the plants fend off colonization by phytopathogenic organisms. These specific interactions between the pathogen and the host determine the course of infection (Schopfer and Brennicke (1999) Pflanzenphysiologie, Springer Verlag, Berlin-Heidelberg, Germany).

With regard to the race specific resistance, also called host resistance, a differentiation is made between compatible and incompatible interactions. In the compatible interaction, an interaction occurs between a virulent pathogen and a susceptible plant. The pathogen survives, and may build up reproduction structures, while the host dies off. An incompatible interaction occurs on the other hand when the pathogen infects the plant but is inhibited in its growth before or after weak development of symptoms. In the latter case, the plant is resistant to the respective pathogen (Schopfer and Brennick, vide supra). In both compatible and incompatible interactions a defensive and specific reaction of the host to the pathogen occurs.

In nature, however, this host resistance is often overcome because of the rapid evolutionary development of pathogens (Neu et al. (2003) American Cytopathol. Society, MPMI 16 No. 7: 626-633). As against this, the non-host resistance offers strong, broad, and permanent protection from phytopathogens. Non-host resistance means the phenomenon that a pathogen can induce a disease in a certain plant species, but not in other plant species (Heath (2002) Can. J. Plant Pathol. 24: 259-264).

Despite this interesting characteristic, the genetic and molecular biological bases for the non-host resistance have up to now only been poorly understood. There are indications that the non-host resistance is induced by unspecific agents, and also that individual pathogen proteins induce the non-host resistance reaction (Heath (1981) Phytopathology 71: 1121-1123; Heath (2001) Physiol. Mol. Plant. Pathol. 58: 53-54; Kamoun et al. (1998) Plant Cell 10: 1413-1425; Lauge et al. (2000) Plant J. 23: 735-745; Whalen et al. (1988) Proc. Natl. Acad. Sci. USA 85: 6743-6747). The phenomenon of non-host resistance might also be based on structural or chemical properties of the plant species, such as the thickness of the cuticle or the presence of inhibitory substances.

Besides resistance based on preformed physical barriers, the most effective non-host defense system of the plant is represented by the recognition of conserved molecular microbial structures, also termed PAMPs (pathogen associated molecular patterns). Recognition of PAMPs by a PAMP-receptor triggers a signaling cascade leading to the activation of a multitude of resistance mechanisms, including cell wall fortification, secretion of toxic compounds and programmed cell death. Those defense mechanisms suffice to effectively arrest the attempted assaults of most microbes. This innate immune response is thought to be an integral part of the genetically complex and durable set of non-host resistance defense mechanisms.

Only a few pathogen species appear to have evolved specific mechanisms to circumvent or to block the basic defense system of individual plant species, and, as a consequence, have become pathogens of these species. It is conceivable that targeting and manipulation of particular host proteins is a key step of this species-specific defense sabotage.

Powdery mildew is a common fungal disease of many monocotyledonous and dicotyledonous plants like beet, various cereals, cucumber, lettuce, carrot, pea, tomato, strawberry, apple, grapes etc. Powdery mildew fungi (Erysiphales) belong to the division of Ascomycota. Blumeria graminis is the fungus that causes powdery mildew in grasses. The barley powdery mildew fungus (Blumeria graminis f. sp. hordei, Bgh) is an obligate biotrophic pathogen that attacks epidermal cells of barley (Hordeum vulgare L.). After contact of the spore with the cuticle of the barley leaf, an appressorium is formed. The following and crucial step of fungal invasion is the penetration of the cell wall followed by the establishment of a specialized intracellular feeding structure called “haustorium” that does not destroy plasma membrane integrity.

In the monocotyledonous barley, the presence of isoforms of the family of heptahelical plasma membrane-localized MLO proteins is required for successful penetration of the host cell wall by the powdery mildew fungi. Absence of these MLO proteins, either caused by natural genetic variations or induced lesions in the respective mlo genes, results in failure of the fungal spores to penetrate the plant cell wall. All barley genotypes lacking a functional mlo gene are resistant to all known isolates of the barley powdery mildew. Additionally, the recessively inherited mlo resistance is extremely durable under field conditions. The mlo mutation has been used in most European spring barley varieties for the last 25 years.

The MLO protein is an integral plasma membrane-localized protein and contains seven hydrophobic transmembrane domains. The cytoplasmic C-terminus harbors an amphiphilic α-helix that serves as a calmodulin binding domain, which is necessary for full activity of the MLO protein. Calcium-dependent calmodulin binding to this peptide domain was shown both in vitro and in vivo and contributes about half of the susceptibility-conferring activity of mlo (Kim M. C. Nature. 2002 Mar. 28; 416(6879):447-51).

The gene ror2 (required for mlo resistance 2) that, when mutated, suppressed mlo-mediated resistance, was found to encode a plasma membrane-resident syntaxin, a protein belonging to the superfamily of SNARE proteins. Lack of wild-type ROR2 partially compromises penetration resistance in mlo genotypes, suggesting that syntaxin activity is required for effective mlo resistance (Freialdenhoven A. Plant Cell. 1996 January; 8(1):5-14). Both MLO and ROR2 seem to focally accumulate at sites of attempted fungal cell-wall penetration. Thus, it appears that MLO and ROR2 form a novel pathogen-triggered micro-domain at biotic stress sites (Bhat R. A. Proc. Natl. Acad. Sci. USA. 2005 Feb. 22; 102(8):3135-40). At these subcellular regions, interaction between MLO and the cytoplasmic calcium sensor calmodulin transiently increases during successful fungal host cell invasion (Bhat R. A., vide supra). Moreover, a direct physical interaction between MLO and ROR2 was suggested. The intensity of this interaction is drastically lowered between a subset of single amino acid substitution mlo mutant proteins and wild-type ROR2 as well as between wild-type MLO and the barley variant encoded by the barley ror2 mutant. MLO might modulate SNARE protein dependent and vesicle transport-associated processes at the plasma membrane. In conclusion, powdery mildew fungi appear to specifically corrupt MLO to modulate vesicle-associated processes at the plant cell periphery for successful pathogenesis.

This mechanism of MLO seems to be conserved in plants. The dicotyledonous plant Arabidopsis thaliana contains 15 homologues of barley MLO, called AtMlo1-AtMlo15. The knockout of AtMlo2 confers resistance to the powdery mildew fungi Erysiphe chichoracearum and Golvinomyces orontii. These data indicate that the powdery mildew infection mechanism is conserved between monocotyledonous and dicotyledonous plants.

It was considered to be plausible that each pathogen species evolved its own specific means to suppress and overcome general or specialized host defense mechanisms. The resistance phenotypes mediated by mlo appear to be highly specific for powdery mildew fungi (Ascomycota-Pezizomycotina-Leotiomycetes-Erysiphales). Up to present it is known from the literature that the mlo mutation does not confer resistance to any pathogen other than powdery mildew fungi.

For example, the mlo mutation does not confer any resistance to other fungi of the division of Ascomycota, which are closely related to powdery mildew fungi. No effect of mlo is observed after inoculation of barley with the take-all fungus (Gaeumannomyces graminis: Ascomycota-Pezizomycotina-Sordariomycetes-Sordariomycetes incertae sedis-Magnaporthaceae; Jorgensen J. H. Induced Mutations Against Plant Diseases, Proc. Symp. Wien, 1977. Wien: Int. Atomic Energy Agency, pp. 533-547). Compared with mlo wild-type plants, barley mlo mutants do not differ in the infection phenotype to a range of other phytopathogens, as for example barley leaf rust (Puccinia striiformis; Basidomycota-Urediniomycetes-Urediniomycetidae-Uredinales-Pucciniaceae) or stripe rust (Puccinia hordei; Basidomycota-Urediniomycetes-Urediniomycetidae-Uredinales-Pucciniaceae).

Moreover the mlo mutation is responsible for an enhanced susceptibility of barley to the hemibiotrophic rice blast fungus Magnaporthe grisea (Ascomycota-Pezizomycotina-Sordariomycetes-Sordariomycetes incertae sedis-Magnaporthaceae; Jarosch B. Mol. Plant-Microbe Interact. 12 (6): 508-514, 1999) and to the necrotrophic fungus Bipolaris sorokiniana (Ascomycota-Pezizomycotina-Dothideomycetes-Pleosporales-Pleosporaceae). In summary, beside powdery mildew fungi, no pathogen is known that is negatively influenced by the mlo mutation.

Soybean rust (SR), also known as Asian soybean rust (Basidiomycota-Urediniomycetes-Urediniomycetidae-Uredinales-Phakopsoraceae), is a disease that affects soybeans and other legumes. It is caused by two types of fungi, Phakopsora pachyrhizi and Phakopsora meibomiae, the latter being the weaker pathogen of the two.

The infection process of soybean rust starts when urediospores germinate to produce a single germ tube, form appressoria and infect always by direct, cuticular penetration. Penetration starts with the formation of an appressorial cone which is continuous with the cell wall of the penetration hypha. The penetration hypha enters the epidermal cell, transverses it and reaches the intercellular space of the mesophyll where the first septum is formed, separating the penetration hypha from the primary hypha. The first haustorium is visible between 24 and 48 hours after inoculation. Haustoria are formed in the mesophyll and epidermal cells.

Under optimal conditions it takes spores 6-7 days to mature. Then, after infection of healthy soybean plants, new spores are produced for about 10 days. These new spores can re-infect the same plant or be carried to other susceptible plants. Soybean rust causes lesions on cotyledons, stems, petioles, leaves, and pods of soybean and other host plants. The main effects on the soybean plant are destruction of photosynthetic tissue which in turn causes premature defoliation, early maturation, and severe yield reductions through reduction in the number of pods and seeds, and decreased seed weight.

Currently there is no resistance to soybean rust in any of the U.S. commercial soybean cultivars. Specific resistance to P. pachyrhizi is known, and four single dominant genes have been identified as Rpp1, Rpp2, Rpp3, and Rpp4. These four genes condition resistance to a limited set of rust isolates. Single gene resistance has not been durable, and the usefulness of the single genes was lost soon after the sources were identified.

The object of the present invention is to provide a method of increasing resistance against soybean rust in transgenic plants and/or plant cells. This object is achieved by the subject-matter of the main claim. The features of the other independent claims serve to solve this and further objects shown in the description. Preferred embodiments of the invention are defined by the features of the sub-claims.

### DETAILED DESCRIPTION OF THE INVENTION

Surprisingly, the inventors found an influence of an mlo mutation in the resistance reaction against soybean rust. This interaction phenotype is totally unexpected because of several reasons:

1. With the exception of the powdery mildew resistance, no other resistance phenotype has been described until now that is based on MLO influence, i.e. MLO overexpression or MLO underexpression. For the fungi Magnaporthe grisea and Bipolaris sorokiniana, even an enhanced susceptibility has been observed in mlo mutants.
2. The phenotype of mlo-barley is indistinguishable from wild type plants after infection with other rust fungi which are closely related to soybean rust. i.e. stripe rust (Puccinia hordei, Basidiomycota; Urediniomycetes; Urediniomycetidae; Uredinales; Pucciniaceae) and barley leaf rust (Puccinia striiformis, Basidiomycota; Urediniomycetes; Urediniomycetidae; Uredinales; Pucciniaceae).
3. Furthermore, MLO does not influence other pathogenic fungi which use infection processes similar to soybean rust, e.g. direct penetration of the epidermal cells and/or intercellular growth in the mesophyll. For example rice blast fungus Magnaporthe oryzae also penetrates directly the epidermis to grow within the leaf.
4. Finally, the powdery mildew fungus is from the division of Ascomycota, whereas the Asian soybean rust is from the division of Basidomycota.

Therefore, in a first aspect, the present invention relates to a method of increasing resistance against soybean rust in transgenic plants and/or plant cells, characterized in that the content and/or the activity of at least one MLO protein is altered in comparison to wild type plants or plant cells, respectively.

Within the scope of the present invention, “transgenic” plant cells (or plants) are cells into which a nucleic acid molecule has been introduced. This molecule can be a DNA/cDNA molecule or an RNA molecule, it can be double-stranded or single-stranded, and examples of such molecules are double-stranded RNA molecules or vectors, e.g. plasmids, cosmids, recombinant viruses or minichromosomes. The nucleic acid molecule can comprise sequences that derive from the species of the host cell or from another organism/species. Furthermore, those sequences can be natural or modified or synthetic.

According to the present invention, a “plant” can be any monocotyledonous or dicotyledonous plant. It is preferably a monocotyledonous or dicotyledonous agricultural, food or feed plant. Preferably, the monocotyledonous plant is selected from the group consisting of Hordeum (barley), Avena (oat), Triticum (wheat), Secale (rye), Oryza (rice), Sorghum (millet), Zea (corn), Panicum, Pennisetum, Setaria and suchlike. Preferably, the dicotyledonous plant is selected from the group consisting of soybean, cotton, rapeseed, tomato, sugar beet, potato, sunflower, pea, ornamental plants, tobacco, clover (Trifolium spec.), Kudzu (Pueraria lobata), trees and legumes such as Alfalfa. Further agricultural plants can include fruit (in particular apples, pears, cherries, grapes, citrus fruits, pineapples and bananas), oil palms, tea, cocoa and coffee trees, tobacco, sisal, as well as medical plants such as rauwolfia and digitales. Particularly favored are the cereals wheat, rye, oat, barley, rice, maize and millet, sugar beet, rape, soybean, tomato, potato and tobacco. Other agricultural plants can be taken from U.S. Pat. No. 6,137,030. The most preferred plant is soybean.

The plant cells according to the invention include differentiated and undifferentiated plant cells including protoplasts which were produced by the method according to the invention and which have integrated the nucleic acid molecules described in the following into the plant genome, or have received these as autonomously replicating molecules.

In the scope of the present invention, the soybean rust pathogen is either Phakopsora pachyrhizi or Phakopsora meibomiae. Preferably, the pathogen is Phakopsora pachyrhizi.

Pathogen “resistance” means the lessening or weakening of a plant's pathogenic symptoms following an attack by a pathogen. The symptoms may be of various kinds, but preferably comprise those which directly or indirectly lead to an impairment of the quality of the plant, the size of the harvest, suitability for use as animal fodder or food for human consumption, or which hamper the sowing, cultivation, harvesting or processing of the crop.

According to the invention, the term “increased resistance” (against soybean rust) is understood to mean that the transgenic plants, or plant cells, according to the invention are less vigorously, and/or less frequently, affected by soybean rust than non-transformed wild type plants, or plant cells, which were otherwise treated in the same way (such as climate and cultivation conditions, pathogen type, etc.). According to the invention, the term “wild type” is to be understood as the respective non genetically modified parent organism. The penetration efficiency as well as the rate of papillae formation offer a possibility to quantify the reaction of the plant to the pathogen infestation (see examples). The term “increased resistance” also comprises what is known as transient pathogen resistance, i.e. the transgenic plants, or plant cells, according to the invention have an increased pathogen resistance as compared to the respective wild type only for a limited period of time.

Transient silencing or transient resistance can be advantageous because it is a valuable addition to other methods which allow the production of plants with increased soybean rust resistance, but which affect the phenotype. Plants which are produced by a method according to the invention and which show transient resistance during development of the infection do not exhibit any significant change of phenotype, and therefore methods according to the invention which give rise to transient resistance can help, along with other methods, to produce plants which are characterized by more enduring and more stable resistance, without having any negative effect upon the phenotype restriction caused by the methods.

The infestation with soybean rust is preferably reduced by at least 10% or 20%, more preferably by at least 30% or 40%, especially preferably by at least 50% or 60%, particularly preferably by at least 70% or 80%, and most preferably by at least 90%, 95% or 100%, which is manifested in a reduction of the development of pathogenic symptoms.

According to the present invention, an “MLO protein” is a protein having an amino acid sequence as depicted in SEQ ID NOs: 2, 4, 7, 9, 11, 14, 16, 18, 20, 22, 24, 26, 28, 30, 32, 34, 36, 38, 40, 42, 44, 46 and 48 or a protein having a sequence which is essentially homologous to said sequences or a protein which is functionally equivalent to said MLO proteins.

The MLO proteins specified by the above-mentioned sequences originate from the following plant species: Hordeum vulgare, Glycine max, Oryza sativa, Linum usitatissimum, Triticum aestivum and Arabidopsis thaliana. However, many other species like Zea mays, Saccharum officinarum, Antirrhinum majus, Solanum tuberosum, Gossypium raimondii, Pinus taeda, Aquilegia Formosa, Aquilegia pubescens, Coffea canephora, Lactuca serriola, Lactuca sativa, Zingiber officinale, Fragaria vesca, Helianthus petiolaris, Brassica rapa, Lotus japonicus, Physcomitrella patens, Capsicum annuum, Lycopersicon esculentum and Nicotiana tabacum contain MLO proteins which are also comprised within the scope of the present invention. The following Table 1 will give an overview of the results of database researches on MLO proteins:

| | | Accession | Patent Number/ |
|---|---|---|---|
| Hit_ID | Organism | Number | GI number |
| FastAlert_N\|EP1586645.43647 | Arabidopsis thaliana | | EP1586645 |
| FastAlert_N\|JP2005185101.15616 | Oryza sativa | | JP2005185101 |
| FastAlert_N\|US2004123343.11552 | Oryza sativa | | US2004123343 |
| FastAlert_N\|US2004214272.113197 | Zea mays | | US2004214272 |
| FastAlert_N\|US2006107345.16980 | | | US2006107345 |
| FastAlert_N\|US2006135758.8510 | | | US2006135758 |
| FastAlert_N\|US2006141495.4465 | | | US2006141495 |
| FastAlert_N\|US2006143729.3731 | | | US2006143729 |
| FastAlert_N\|US2006150283.101384 | | | US2006150283 |
| FastAlert_N\|US6680427.1 | | | U.S. Pat. No. 6,680,427 |
| GENBANK_EST2\|BX837230 | Arabidopsis thaliana | BX837230 | 42531313 |
| GENBANK_EST2\|CA084154 | Saccharum | CA084154 | 34937465 |
| | officinarum | | |
| GENBANK_EST2\|CB642264 | Oryza sativa | CB642264 | 29637255 |
| | (japonica cultivar- | | |
| | group) | | |
| GENBANK_EST3\|AJ803024 | Antirrhinum majus | AJ803024 | 51118352 |
| GENBANK_EST3\|CK276064 | Solanum tuberosum | CK276064 | 39833042 |
| GENBANK_EST3\|CO085008 | Gossypium | CO085008 | 48775642 |
| | raimondii | | |
| GENBANK_EST4\|DR092880 | Pinus taeda | DR092880 | 67551853 |
| GENBANK_EST4\|DR801464 | Zea mays | DR801464 | 71329486 |
| GENBANK_EST4\|DR917775 | Aquilegia formosa x | DR917775 | 71687138 |
| | Aquilegia pubescens | | |
| GENBANK_EST4\|DT755246 | Aquilegia formosa x | DT755246 | 74554469 |
| | Aquilegia pubescens | | |
| GENBANK_EST4\|DV705346 | Coffea canephora | DV705346 | 82485174 |
| GENBANK_EST4\|DV707424 | Coffea canephora | DV707424 | 82487252 |
| GENBANK_EST4\|DV710345 | Coffea canephora | DV710345 | 82490173 |
| GENBANK_EST4\|DW118461 | Lactuca serriola | DW118461 | 83916381 |
| GENBANK_EST5\|DY356131 | Zingiber officinale | DY356131 | 87089344 |
| GENBANK_EST5\|DY669424 | Fragaria vesca | DY669424 | 89545769 |
| GENBANK_EST5\|DY943422 | Helianthus petiolaris | DY943422 | 90481564 |
| GENBANK_EST5\|DY945447 | Helianthus petiolaris | DY945447 | 90483589 |
| GENBANK_EST5\|DY950977 | Helianthus petiolaris | DY950977 | 90489119 |
| GENBANK_EST5\|DY963085 | Lactuca sativa | DY963085 | 90501227 |
| GENBANK_EST5\|EB425411 | Nicotiana tabacum | EB425411 | 92011825 |
| GENBANK_EST5\|EB441983 | Nicotiana tabacum | EB441983 | 92030278 |
| GENBANK_GSS2\|CL945757 | Oryza sativa (indica | CL945757 | 52357766 |
| | cultivar-group) | | |
| GENBANK_GSS2\|CL964365 | Oryza sativa (indica | CL964365 | 52383436 |
| | cultivar-group) | | |
| GENBANK_GSS2\|CL966901 | Oryza sativa (indica | CL966901 | 52388451 |
| | cultivar-group) | | |
| GENBANK_GSS2\|CL969027 | Oryza sativa (indica | CL969027 | 52392684 |
| | cultivar-group) | | |
| GENBANK_GSS2\|CL969945 | Oryza sativa (indica | CL969945 | 52394507 |
| | cultivar-group) | | |
| GENBANK_GSS2\|CL970460 | Oryza sativa (indica | CL970460 | 52395529 |
| | cultivar-group) | | |
| GENBANK_GSS2\|CL971387 | Oryza sativa (indica | CL971387 | 52397377 |
| | cultivar-group) | | |
| GENBANK_GSS2\|CL978285 | Oryza sativa (indica | CL978285 | 52411073 |
| | cultivar-group) | | |
| GENBANK_HTC\|AY103929 | Zea mays | AY103929 | 21207007 |
| GENBANK_HTC\|AY104078 | Zea mays | AY104078 | 21207156 |
| GENBANK_HTC\|AY105309 | Zea mays | AY105309 | 21208387 |
| GENBANK_HTC\|AY108340 | Zea mays | AY108340 | 21211418 |
| GENBANK_HTC\|BX819297 | Arabidopsis thaliana | BX819297 | 42469457 |
| GENBANK_HTC\|BX819596 | Arabidopsis thaliana | BX819596 | 42466898 |
| GENBANK_HTC\|BX820553 | Arabidopsis thaliana | BX820553 | 42469164 |
| GENBANK_HTC\|BX820977 | Arabidopsis thaliana | BX820977 | 42469308 |
| GENBANK_HTC\|BX826472 | Arabidopsis thaliana | BX826472 | 42459842 |
| GENBANK_HTC\|BX827067 | Arabidopsis thaliana | BX827067 | 42462173 |
| GENBANK_HTC\|BX831968 | Arabidopsis thaliana | BX831968 | 42458075 |
| GENBANK_HTC\|BX832580 | Arabidopsis thaliana | BX832580 | 42459139 |
| GENBANK_HTC\|BX841625 | Arabidopsis thaliana | BX841625 | 42406472 |
| GENBANK\|A92828 | Hordeum vulgare | A92828 | 6741365 |
| GENBANK\|A92837 | Oryza sativa | A92837 | 6741373 |
| GENBANK\|A92838 | Hordeum vulgare | A92838 | 6741374 |
| GENBANK\|AF361932 | Triticum aestivum | AF361932 | 15290588 |
| | | | 15290589 |
| GENBANK\|AF361933 | Triticum aestivum | AF361933 | 15290590 |
| | | | 15290591 |
| GENBANK\|AF369563 | Arabidopsis thaliana | AF369563 | 14091573 |
| | | | 14091574 |
| GENBANK\|AF369565 | Arabidopsis thaliana | AF369565 | 14091577 |
| | | | 14091578 |
| GENBANK\|AF369566 | Arabidopsis thaliana | AF369566 | 14091579 |
| | | | 14091580 |
| GENBANK\|AF369568 | Arabidopsis thaliana | AF369568 | 14091583 |
| | | | 14091584 |
| GENBANK\|AF369569 | Arabidopsis thaliana | AF369569 | 14091585 |
| | | | 14091586 |
| GENBANK\|AF369572 | Arabidopsis thaliana | AF369572 | 14091591 |
| | | | 14091592 |
| GENBANK\|AF369573 | Arabidopsis thaliana | AF369573 | 14091593 |
| | | | 14091594 |
| GENBANK\|AF369574 | Arabidopsis thaliana | AF369574 | 14091595 |
| | | | 14091596 |
| GENBANK\|AF384030 | Oryza sativa (indica | AF384030 | 15290604 |
| | cultivar-group) | | 15290605 |
| GENBANK\|AF384144 | Triticum aestivum | AF384144 | 14334166 |
| | | | 14334167 |
| GENBANK\|AF384145 | Triticum aestivum | AF384145 | 14334168 |
| | | | 14334169 |
| GENBANK\|AF388195 | Oryza sativa (indica | AF388195 | 14718603 |
| | cultivar-group) | | 14718604 |
| GENBANK\|AK066134 | Oryza sativa | AK066134 | 32976152 |
| | (japonica cultivar- | | |
| | group) | | |
| GENBANK\|AK072272 | Oryza sativa | AK072272 | 32982295 |
| | (japonica cultivar- | | |
| | group) | | |
| GENBANK\|AK072733 | Oryza sativa | AK072733 | 32982756 |
| | (japonica cultivar- | | |
| | group) | | |
| GENBANK\|AK098993 | Oryza sativa | AK098993 | 32984202 |
| | (japonica cultivar- | | |
| | group) | | |
| GENBANK\|AK111990 | Oryza sativa | AK111990 | 37988653 |
| | (japonica cultivar- | | |
| | group) | | |
| GENBANK\|AK121347 | Oryza sativa | AK121347 | 37990970 |
| | (japonica cultivar- | | |
| | group) | | |
| GENBANK\|AK121374 | Oryza sativa | AK121374 | 37990997 |
| | (japonica cultivar- | | |
| | group) | | |
| GENBANK\|AK221618 | Arabidopsis thaliana | AK221618 | 62320583 |
| | | | 62320584 |
| GENBANK\|AR172598 | Unknown. | AR172598 | 17912089 |
| GENBANK\|AR172601 | Unknown. | AR172601 | 17912092 |
| GENBANK\|AR172602 | Unknown. | AR172602 | 17912093 |
| GENBANK\|AR172603 | Unknown. | AR172603 | 17912094 |
| GENBANK\|AR454293 | Unknown. | AR454293 | 42687440 |
| GENBANK\|AR633457 | Unknown. | AR633457 | 59780849 |
| GENBANK\|AR633459 | Unknown. | AR633459 | 59780853 |
| GENBANK\|AR633462 | Unknown. | AR633462 | 59780859 |
| GENBANK\|AR633469 | Unknown. | AR633469 | 59780872 |
| GENBANK\|AX063294 | Triticum sp. | AX063294 | 12541084 |
| | | | 12541085 |
| GENBANK\|AX063296 | Triticum sp. | AX063296 | 12541086 |
| | | | 12541087 |
| GENBANK\|AX063298 | Triticum sp. | AX063298 | 12541088 |
| | | | 12541089 |
| GENBANK\|AX063300 | Arabidopsis thaliana | AX063300 | 12541090 |
| | | | 12541091 |
| GENBANK\|AX063302 | Arabidopsis thaliana | AX063302 | 12541092 |
| | | | 12541093 |
| GENBANK\|AX063304 | Arabidopsis thaliana | AX063304 | 12541094 |
| | | | 12541095 |
| GENBANK\|AX063306 | Arabidopsis thaliana | AX063306 | 12541096 |
| | | | 12541097 |
| GENBANK\|AX063308 | Arabidopsis thaliana | AX063308 | 12541098 |
| | | | 12541099 |
| GENBANK\|AX412295 | Arabidopsis thaliana | AX412295 | 21444753 |
| GENBANK\|AX506391 | Arabidopsis thaliana | AX506391 | 23387628 |
| GENBANK\|AX506652 | Arabidopsis thaliana | AX506652 | 23387889 |
| GENBANK\|AX506994 | Arabidopsis thaliana | AX506994 | 23388231 |
| GENBANK\|AX507353 | Arabidopsis thaliana | AX507353 | 23388590 |
| GENBANK\|AX507573 | Arabidopsis thaliana | AX507573 | 23388810 |
| GENBANK\|AX653006 | Oryza sativa | AX653006 | 29155820 |
| GENBANK\|AX653229 | Oryza sativa | AX653229 | 29156043 |
| GENBANK\|AX653497 | Oryza sativa | AX653497 | 29156311 |
| GENBANK\|AX653740 | Oryza sativa | AX653740 | 29156554 |
| GENBANK\|AX654786 | Oryza sativa | AX654786 | 29157600 |
| GENBANK\|AY029312 | Zea mays | AY029312 | 44458501 |
| | | | 44458502 |
| GENBANK\|AY029313 | Zea mays | AY029313 | 13784976 |
| | | | 13784977 |
| GENBANK\|AY029314 | Zea mays | AY029314 | 13784978 |
| | | | 13784979 |
| GENBANK\|AY029315 | Zea mays | AY029315 | 13784980 |
| | | | 13784981 |
| GENBANK\|AY029317 | Zea mays | AY029317 | 13784984 |
| | | | 13784985 |
| GENBANK\|AY029318 | Zea mays | AY029318 | 13784986 |
| | | | 13784987 |
| GENBANK\|AY029319 | Zea mays | AY029319 | 13784988 |
| | | | 13784989 |
| GENBANK\|AY029320 | Zea mays | AY029320 | 13784990 |
| | | | 13784991 |
| GENBANK\|AY054241 | Arabidopsis thaliana | AY054241 | 15809945 |
| | | | 15809946 |
| GENBANK\|AY057502 | Arabidopsis thaliana | AY057502 | 15982790 |
| | | | 15982791 |
| GENBANK\|AY072135 | Arabidopsis thaliana | AY072135 | 18175952 |
| | | | 18175953 |
| GENBANK\|AY086586 | Arabidopsis thaliana | AY086586 | 21405296 |
| | | | 21554658 |
| GENBANK\|AY113992 | Arabidopsis thaliana | AY113992 | 21280824 |
| | | | 21280825 |
| GENBANK\|AY581255 | Hordeum vulgare | AY581255 | 46405142 |
| | subsp. vulgare | | 46405143 |
| GENBANK\|AY584534 | Triticum aestivum | AY584534 | 46405854 |
| | | | 46405855 |
| GENBANK\|AY599871 | Physcomitrella | AY599871 | 47028562 |
| | patens | | 47028563 |
| GENBANK\|AY934528 | Capsicum annuum | AY934528 | 60617256 |
| | | | 60617257 |
| GENBANK\|AY967408 | Lycopersicon | AY967408 | 62208138 |
| | esculentum | | 62208139 |
| GENBANK\|AY967409 | Brassica rapa | AY967409 | 62208140 |
| | | | 62208141 |
| GENBANK\|AY967410 | Lotus japonicus | AY967410 | 62208142 |
| | | | 62208143 |
| GENBANK\|BT000434 | Arabidopsis thaliana | BT000434 | 23306367 |
| | | | 23306368 |
| GENBANK\|BT002581 | Arabidopsis thaliana | BT002581 | 27311950 |
| | | | 27311951 |
| GENBANK\|BT002918 | Arabidopsis thaliana | BT002918 | 27754573 |
| | | | 27754574 |
| GENBANK\|BT004356 | Arabidopsis thaliana | BT004356 | 28393884 |
| | | | 28393885 |
| GENBANK\|BT009442 | Triticum aestivum | BT009442 | 32128993 |
| GENBANK\|BT010322 | Arabidopsis thaliana | BT010322 | 33942040 |
| | | | 33942041 |
| GENBANK\|DW486556 | | | |
| GENBANK\|Z83834 | Hordeum vulgare | Z83834 | 1877220 1877221 |
| | subsp. vulgare | | |
| GENBANK\|Z95352 | Arabidopsis thaliana | Z95352 | 2765816 2765817 |
| GENESEQ_DNA\|AAA52708 | Triticum aestivum. | AAA52708 | WO200036110 |
| GENESEQ_DNA\|AAA52715 | Triticum aestivum. | AAA52715 | WO200036110 |
| GENESEQ_DNA\|AAA52718 | Triticum aestivum. | AAA52718 | WO200036110 |
| GENESEQ_DNA\|AAC44660 | Arabidopsis | AAC44660 | EP1033405 |
| | thaliana. | | |
| GENESEQ_DNA\|AAF24583 | Triticum sp. | AAF24583 | WO200078799 |
| GENESEQ_DNA\|AAF24584 | Triticum sp. | AAF24584 | WO200078799 |
| GENESEQ_DNA\|AAF24585 | Triticum sp. | AAF24585 | WO200078799 |
| GENESEQ_DNA\|AAF24586 | Arabidopsis | AAF24586 | WO200078799 |
| | thaliana. | | |
| GENESEQ_DNA\|AAF24587 | Arabidopsis | AAF24587 | WO200078799 |
| | thaliana. | | |
| GENESEQ_DNA\|AAF24588 | Arabidopsis | AAF24588 | WO200078799 |
| | thaliana. | | |
| GENESEQ_DNA\|AAF24589 | Arabidopsis | AAF24589 | WO200078799 |
| | thaliana. | | |
| GENESEQ_DNA\|AAF24590 | Arabidopsis | AAF24590 | WO200078799 |
| | thaliana. | | |
| GENESEQ_DNA\|AAS01109 | Zea mays. | AAS01109 | US6211433 |
| GENESEQ_DNA\|AAV35022 | Hordeum vulgare. | AAV35022 | WO9804586 |
| GENESEQ_DNA\|AAV35026 | Hordeum vulgare. | AAV35026 | WO9804586 |
| GENESEQ_DNA\|AAV35028 | Oryza sativa. | AAV35028 | WO9804586 |
| GENESEQ_DNA\|AAV35030 | Hordeum vulgare. | AAV35030 | WO9804586 |
| GENESEQ_DNA\|AAV35031 | Arabidopsis | AAV35031 | WO9804586 |
| | thaliana. | | |
| GENESEQ_DNA\|AAX58270 | Zea mays. | AAX58270 | WO9923235 |
| GENESEQ_DNA\|AAX58273 | Zea mays. | AAX58273 | WO9923235 |
| GENESEQ_DNA\|AAX58274 | Zea mays. | AAX58274 | WO9923235 |
| GENESEQ_DNA\|AAX58275 | Zea mays. | AAX58275 | WO9923235 |
| GENESEQ_DNA\|AAZ30409 | Triticum sp. | AAZ30409 | WO9947552 |
| GENESEQ_DNA\|AAZ30410 | Triticum sp. | AAZ30410 | WO9947552 |
| GENESEQ_DNA\|AAZ30411 | Triticum sp. | AAZ30411 | WO9947552 |
| GENESEQ_DNA\|AAZ30412 | Arabidopsis | AAZ30412 | WO9947552 |
| | thaliana. | | |
| GENESEQ_DNA\|AAZ30413 | Arabidopsis | AAZ30413 | WO9947552 |
| | thaliana. | | |
| GENESEQ_DNA\|AAZ30414 | Arabidopsis | AAZ30414 | WO9947552 |
| | thaliana. | | |
| GENESEQ_DNA\|AAZ30415 | Arabidopsis | AAZ30415 | WO9947552 |
| | thaliana. | | |
| GENESEQ_DNA\|AAZ30416 | Arabidopsis | AAZ30416 | WO9947552 |
| | thaliana. | | |
| GENESEQ_DNA\|AAZ49561 | Zea mays. | AAZ49561 | WO200001722 |
| GENESEQ_DNA\|AAZ49562 | Zea mays. | AAZ49562 | WO200001722 |
| GENESEQ_DNA\|AAZ49564 | Zea mays. | AAZ49564 | WO200001722 |
| GENESEQ_DNA\|AAZ49565 | Zea mays. | AAZ49565 | WO200001722 |
| GENESEQ_DNA\|AAZ49566 | Zea mays. | AAZ49566 | WO200001722 |
| GENESEQ_DNA\|AAZ49567 | Zea mays. | AAZ49567 | WO200001722 |
| GENESEQ_DNA\|AAZ50126 | Zea mays. | AAZ50126 | WO200001721 |
| GENESEQ_DNA\|ABZ13281 | Arabidopsis | ABZ13281 | WO200216655 |
| | thaliana. | | |
| GENESEQ_DNA\|ABZ13542 | Arabidopsis | ABZ13542 | WO200216655 |
| | thaliana. | | |
| GENESEQ_DNA\|ABZ13875 | Arabidopsis | ABZ13875 | WO200216655 |
| | thaliana. | | |
| GENESEQ_DNA\|ABZ13884 | Arabidopsis | ABZ13884 | WO200216655 |
| | thaliana. | | |
| GENESEQ_DNA\|ABZ14243 | Arabidopsis | ABZ14243 | WO200216655 |
| | thaliana. | | |
| GENESEQ_DNA\|ABZ14463 | Arabidopsis | ABZ14463 | WO200216655 |
| | thaliana. | | |
| GENESEQ_DNA\|ADA67959 | Arabidopsis | ADA67959 | WO2003000898 |
| | thaliana. | | |
| GENESEQ_DNA\|ADA68054 | Arabidopsis | ADA68054 | WO2003000898 |
| | thaliana. | | |
| GENESEQ_DNA\|ADA69553 | Oryza sativa. | ADA69553 | WO2003000898 |
| GENESEQ_DNA\|ADA69776 | Oryza sativa. | ADA69776 | WO2003000898 |
| GENESEQ_DNA\|ADA70044 | Oryza sativa. | ADA70044 | WO2003000898 |
| GENESEQ_DNA\|ADA70287 | Oryza sativa. | ADA70287 | WO2003000898 |
| GENESEQ_DNA\|ADA71333 | Oryza sativa. | ADA71333 | WO2003000898 |
| GENESEQ_DNA\|ADG87617 | Arabidopsis | ADG87617 | WO200222675 |
| | thaliana. | | |
| GENESEQ_DNA\|ADG87618 | Arabidopsis | ADG87618 | WO200222675 |
| | thaliana. | | |
| GENESEQ_DNA\|ADT16339 | Viridiplantae. | ADT16339 | US2004216190 |
| GENESEQ_DNA\|ADT18635 | Viridiplantae. | ADT18635 | US2004216190 |
| GENESEQ_DNA\|ADX12455 | Unidentified. | ADX12455 | US2004034888 |
| GENESEQ_DNA\|ADX27198 | Unidentified. | ADX27198 | US2004034888 |
| GENESEQ_DNA\|ADX30090 | Unidentified. | ADX30090 | US2004034888 |
| GENESEQ_DNA\|ADX31306 | Unidentified. | ADX31306 | US2004034888 |
| GENESEQ_DNA\|ADX46115 | Unidentified. | ADX46115 | US2004034888 |
| GENESEQ_DNA\|ADX47477 | Unidentified. | ADX47477 | US2004034888 |
| GENESEQ_DNA\|ADX54605 | Unidentified. | ADX54605 | US2004034888 |
| GENESEQ_DNA\|ADX59361 | Unidentified. | ADX59361 | US2004034888 |
| GENESEQ_DNA\|ADX62039 | Unidentified. | ADX62039 | US2004034888 |
| GENESEQ_DNA\|ADX62042 | Unidentified. | ADX62042 | US2004034888 |
| GENESEQ_DNA\|ADX63313 | Unidentified. | ADX63313 | US2004034888 |
| GENESEQ_DNA\|AEH11765 | Hordeum vulgare. | AEH11765 | WO2006042145 |
| GENESEQ_DNA\|AEH11766 | Oryza sativa. | AEH11766 | WO2006042145 |
| Hordeum_vulgare_Barley_Apr_03\|c62774660hv270303 | | | |
| Hyseq_Canola_Oct02\|bn1106c25867 | | | |
| Hyseq_Canola_Oct02\|bn1106c25990 | | | |
| REFSEQ_NUCLEOTIDE\|NM_001036501 | Arabidopsis thaliana | NM_001036501 | 79324986 |
| REFSEQ_NUCLEOTIDE\|NM_001036993 | Arabidopsis thaliana | NM_001036993 | 79330794 |
| REFSEQ_NUCLEOTIDE\|NM_100975 | Arabidopsis thaliana | NM_100975 | 30682023 |
| REFSEQ_NUCLEOTIDE\|NM_101004 | Arabidopsis thaliana | NM_101004 | 18391262 |
| REFSEQ_NUCLEOTIDE\|NM_102433 | Arabidopsis thaliana | NM_102433 | 18396018 |
| REFSEQ_NUCLEOTIDE\|NM_103440 | Arabidopsis thaliana | NM_103440 | 79358659 |
| REFSEQ_NUCLEOTIDE\|NM_104836 | Arabidopsis thaliana | NM_104836 | 18407233 |
| REFSEQ_NUCLEOTIDE\|NM_114398 | Arabidopsis thaliana | NM_114398 | 18407954 |
| REFSEQ_NUCLEOTIDE\|NM_116494 | Arabidopsis thaliana | NM_116494 | 30679208 |
| REFSEQ_NUCLEOTIDE\|NM_118558 | Arabidopsis thaliana | NM_118558 | 42567096 |
| REFSEQ_NUCLEOTIDE\|NM_124755 | Arabidopsis thaliana | NM_124755 | 30696372 |
| REFSEQ_NUCLEOTIDE\|NM_125994 | Arabidopsis thaliana | NM_125994 | 18425014 |
| REFSEQ_NUCLEOTIDE\|NM_127298 | Arabidopsis thaliana | NM_127298 | 42569101 |
| REFSEQ_NUCLEOTIDE\|NM_127302 | Arabidopsis thaliana | NM_127302 | 30679992 |
| REFSEQ_NUCLEOTIDE\|NM_128925 | Arabidopsis thaliana | NM_128925 | 18403339 |
| REFSEQ_NUCLEOTIDE\|NM_129478 | Arabidopsis thaliana | NM_129478 | 30687810 |
| REFSEQ_NUCLEOTIDE\|NM_129974 | Arabidopsis thaliana | NM_129974 | 18406453 |
| REFSEQ_NUCLEOTIDE\|NM_190204 | Oryza sativa | NM_190204 | 34907491 |
| | (japonica cultivar- | | |
| | group) | | |
| REFSEQ_NUCLEOTIDE\|NM_197580 | Oryza sativa | NM_197580 | 37536519 |
| | (japonica cultivar- | | |
| | group) | | |
| REFSEQ_NUCLEOTIDE\|NM_201957 | Arabidopsis thaliana | NM_201957 | 42571224 |
| REFSEQ_NUCLEOTIDE\|XM_464475 | Oryza sativa | XM_464475 | 50905972 |
| | (japonica cultivar- | | |
| | group) | | |
| REFSEQ_NUCLEOTIDE\|XM_472638 | Oryza sativa | XM_472638 | 50924555 |
| | (japonica cultivar- | | |
| | group) | | |
| REFSEQ_NUCLEOTIDE\|XM_474381 | Oryza sativa | XM_474381 | 50929706 |
| | (japonica cultivar- | | |
| | group) | | |
| REFSEQ_NUCLEOTIDE\|XM_493809 | Oryza sativa | XM_493809 | 50948902 |
| | (japonica cultivar- | | |
| | group) | | |
| Triticum_aestivum_Wheat_Apr03\|c55126395 | | | |

In the scope of the present invention, the term “homologous” is used in reference to amino acid sequences or nucleic acid sequences, meaning that they share a certain degree of “homology”, i.e. “identity” or “similarity”, with another amino acid sequence or nucleic acid sequence, respectively.

Many algorithms exist to determine this degree of homology or similarity. Preferably the homology can be determined by means of the Lasergene software of the company DNA star Inc., Madison, Wis. (USA), using the CLUSTAL method (Higgins et al., 1989, Comput. Appl. Biosci., 5 (2), 151). Other programs that a skilled person can use for the comparison of sequences and that are based on algorithms are, e.g., the algorithms of Needleman and Wunsch or Smith and Waterman. Further useful programs are the Pile Aupa program (J. Mol. Evolution. (1987), 25, 351-360; Higgins et al., (1989), Cabgos, 5, 151-153) or the Gap and Best Fit program (Needleman and Wunsch, (1970), J. Mol. Biol., 48, 443-453, as well as Smith and Waterman (1981), Adv., Appl. Math., 2, 482-489) or the programs of the GCG software package of the Genetics Computer Group (575 Science Drive, Madison, Wis., USA 53711). Sequence alignments can also be performed with the Clustal W program from the internet page http://www.ebi.ac.uk/clustalw or with the NCBI Blast Sequence alignment program from the internet page http://www.ncbi.nlm.nih.gov/BLAST/ or http://www.ncbi.nlm.nih.gov/blast/bl2seq/wblast2.cgi.

The skilled person can find adequate nucleic or amino acid sequences in databases which are available in the internet, e.g. http://www.ncbi.nlm.nih.gov/entrez or http://www.tigr.org. In addition to the known MLO sequences, which are disclosed in the present invention, further sequences can be found in those databases in the future and can be used in the context of the present invention. Also, the skilled person is aware of the techniques which allow him to isolate homologous sequences from other organisms. He can perform homology comparisons (via CLUSTAL, BLAST, NCBI) and then isolate the identified homologous nucleotide sequences by means of standard laboratory methods, e.g. primer design, PCR, hybridization or screening of cDNA libraries with adequate probes (cf. e.g. Sambrook and Russell (2001) Molecular Cloning: A Laboratory Manual, 3^{rd} edition, Cold Spring Harbour Laboratory Press, Cold Spring Harbour, N.Y.). The function of the identified proteins can then be determined.

An amino acid sequence which is “essentially homologous” (=essentially similar) to an MLO amino acid sequence means, in the scope of the present invention, that the sequence is at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% similar to the amino acid sequence of any of the MLO proteins depicted in SEQ ID NOs: 2, 4, 7, 9, 11, 14, 16, 18, 20, 22, 24, 26, 28, 30, 32, 34, 36, 38, 40, 42, 44, 46 and 48 or their functionally equivalent parts or fragments. Preferably, the homology is determined over the whole sequence length of those proteins. The same definition applies analogously to a nucleic acid sequence.

If some of the above-mentioned amino acid sequences are only partial sequences of the full length MLO protein (such as SEQ ID NO: 7), the term “essentially homologous amino acid sequence” also refers to the full length sequence or to new parts of the full length sequence which can be identified in the future.

According to the present invention, a protein which is “functionally equivalent” to an MLO protein is a protein which has the same cellular functions, the same binding properties and/or the same structural properties as any of the MLO proteins having an amino acid sequence as depicted in SEQ ID NOs: 2, 4, 7, 9, 11, 14, 16, 18, 20, 22, 24, 26, 28, 30, 32, 34, 36, 38, 40, 42, 44, 46 and 48. The cellular functions are especially meant to be the interactions of the protein with its pathogenic or physiologic binding partners, i.e. in the case of the MLO proteins their interactions with calmodulin and/or ROR2. Another possible physiologic binding partner of the MLO protein is the ATPase protein family. Any other possible interaction partner which will be discovered in the future is also meant to be included within the scope of the present invention. A functionally equivalent protein can also be a part (fragment) of said MLO proteins, e.g. a protein having amino acid deletions or additions at the N-terminus and/or at the C-terminus. It can also be a protein having one or more amino acid exchanges, insertions or deletions which do not lead to altered cellular functions, binding properties and/or structural properties.

Functional point mutations are, e.g., achieved by a conservative amino acid exchange, i.e. an amino acid is exchanged for another which has comparable physicochemical properties, such as hydrophobic, hydrophilic, positively charged, negatively charged amino acids etc. One example for a conservative amino acid exchange is the replacement of valine for alanine (or vice versa). The skilled person has to keep in mind the region where the exchange is being realized, i.e. if it is a region which is essential for the interaction of the MLO protein with its binding partners. For example, the MLO C-terminus comprises a calmodulin binding site. A sequence alignment with known MLO sequences can give an indication whether a region is essential for the binding behavior of the protein. In contrast to a conservative amino acid exchange, the skilled person will assume that the exchange of, e.g. a positively charged amino acid for a negatively charged amino acid (for example lysine—glutamic acid) will lead to a functional or structural change of the MLO protein. The same considerations apply to the generation of functional insertion or deletion mutants of mlo. The skilled person will pay attention to the issue whether the inserted or deleted amino acids or amino acid ranges are located within a region which is or is not essential for the binding properties of Mlo.

In the context of the present invention, a “fragment” of an MLO is a part of an MLO protein, wherein the original MLO protein has e.g. an amino acid sequence as depicted in SEQ ID NOs: 2, 4, 7, 9, 11, 14, 16, 18, 20, 22, 24, 26, 28, 30, 32, 34, 36, 38, 40, 42, 44, 46 and 48. Usually, the fragment lacks amino acids at the N-terminus or the C-terminus.

In the case that the MLO fragment needs to be functional, the fragment has preferably at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% of the length of the “whole” MLO protein.

Some of the methods described below, however, don't require that the nucleic acid sequence encoding a “fragment” of an MLO protein has to encode a functional protein. In those cases, the “fragment” or “part” of the nucleic acid can be as short as 20 nucleotides, in some cases even shorter. Details of the length of the (amino acid or nucleic acid) fragments will be described below.

Preferably, the MLO used for the present invention is a plant MLO, more preferably a plant MLO selected from the group consisting of Hordeum vulgare (barley) MLO, Oryza sativa (rice) MLO, Arabidopsis thaliana MLO, especially preferably AtMlo1, AtMlo2, AtMlo3, AtMlo4, AtMlo5, AtMlo6, AtMlo7, AtMlo8, AtMlo9, AtMlo10, AtMlo11, AtMlo12, AtMlo13, AtMlo14 or AtMlo15, Linum usitatissimum (flax) MLO, Triticum aestivum (wheat) MLO, Glycine max (soy) Mlo, especially preferably GmMlo1, GmMlo2, GmMlo3.1 or GmMlo3.2, or an MLO which is essentially functionally equivalent to any one of said MLO proteins.

Particularly preferably, the MLO used for the present invention is an MLO selected from the group consisting of an MLO having an amino acid sequence as depicted in any of SEQ ID NOs: 2, 4, 7, 9, 11, 14, 16, 18, 20, 22, 24, 26, 28, 30, 32, 34, 36, 38, 40, 42, 44, 46 and 48, or an MLO having an amino acid sequence which is essentially functionally equivalent to any of the MLO sequences depicted in SEQ ID NOs: 2, 4, 7, 9, 11, 14, 16, 18, 20, 22, 24, 26, 28, 30, 32, 34, 36, 38, 40, 42, 44, 46 and 48.

In the scope of the present invention, the “content” of an MLO protein is considered to be the amount of MLO protein as it can be determined for the wild type of a plant or plant cell with and/or without pathogen inoculation. Several methods which are appropriate to determine the amount of MLO in plant cells will be described in the following. All of those techniques are routine laboratory methods well-known to the skilled person. The exact protocols can be learned from any standard laboratory textbook.

The amount of MLO RNA (being an indirect indication for the protein amount) can be determined by means of a Reverse Transcriptase PCR (RT-PCR): 1. Isolation of total RNA. 2. Reverse transcription to cDNA using poly-T-Primer or random hexamer Primer. 3. PCR with Mlo specific primers using cDNA as Template. (Or One-step-RT-PCR using QuiagenKit).

Another possibility to quantify the amount of MLO RNA is the Northern Blot technique. This method is based on the transfer of electrophoretically separated RNA molecules from a gel onto an absorbent sheet, which is then immersed in a labeled probe that will hybridize to an RNA of interest to reveal its presence.

The amount of protein can be determined by means of the Western Blot (immunoblot) technique: This is a method to detect protein in a given sample of tissue homogenate or extract. It uses gel electrophoresis to separate denatured proteins by mass. The proteins are then transferred out of the gel and onto a membrane (typically nitrocellulose), where they are “probed” using antibodies specific to the protein.

Immunohistochemical staining is also a valuable tool for detecting specific antigens in tissues. In order to perform the standard staining procedure, first the tissue section has to be deparaffinized and then rehydrated before applying the primary antibody. Enzyme-conjugated secondary antibodies are then applied and the specific staining can be visualized after adding the enzyme-specific substrate. Occasionally, when weak or no staining is observed, an antigen “unmasking” by enzyme digestion, may be required.

The “activity” of an MLO protein means its capacity to perform its cellular function, especially the interaction with its physiologic or pathogenic binding partners, most especially with ROR2 and/or calmodulin. The interaction of MLO with Calmodulin and/or Ror2 can be detected using standard protein-protein interaction methods. Calmodulin was shown to interact with MLO in the yeast-two-hybrid (split-ubiquitin) system (see Kim M. C. Journal of Biological Chemistry. 277(22):19304-19314, 2002 May 31; and Kim M. C. Nature. 416(6879):447-450, 2002 Mar. 28). Additionally the interaction was shown in a GST-pulldown experiment (see Kim M. C. Nature. 416(6879):447-450, 2002 Mar. 28). The interaction of the ROR2 syntaxin and MLO was shown by FRET (see Bhat R. A. Proceedings of the National Academy of Sciences of the United States of America. 102(8):3135-3140, 2005 Feb. 22).

Other methods to detect protein-protein interactions can also be applied to MLO and its binding partners. The split-ubiquitin system is an appropriate system to find new interaction partners (as described in Kim et al. 2002, vide supra), but also a transient transformation based (high throughput) FRET or Bi-Fluorescence Complementation (BiFC) screen can be used. In both cases the MLO bait is fused to a fluorescent protein (YFP f.1. for FRET and the N or C terminal half of YFP for BiFC). For the prey, a cDNA library is fused to CFP (FRET) or the complementary half of YFP (BiFC). Bait and prey are co-expressed transiently in epidermis cells or protoplasts. Flourecence is measured by CLSM or fluorescence photometer.

When the content and/or the activity of an MLO protein is altered within a plant or a plant cell, it can be either decreased or increased in comparison to the wild type. The increase of the content of MLO can be achieved by an increase of the endogenous MLO amount (i.e. the MLO that is or by the introduction of an additional amount of MLO into the plants or plant cells. The decrease of the MLO content in the plants or plant cells according to the invention is generally achieved by the decrease of the endogenous MLO amount. Accordingly, the increase of the MLO activity can be achieved by an increase of the endogenous MLO activity and/or the introduction of an additional amount of functional MLO. A decrease of the MLO activity can be achieved by the decrease of the activity of the endogenous MLO. Likewise, a decrease of the MLO activity can also mean that the activity of the endogenous MLO is unmodified, but its interaction with physiologic or pathogenic binding partners is inhibited, e.g. via the expression of a non-functional MLO or of an anti-MLO antibody or an MLO inhibitor. In other words, the interaction of an MLO protein with its binding partners is essentially suppressed and/or substantially prevented.

Accordingly, a preferred embodiment of the present invention is directed to a method of increasing resistance against soybean rust in transgenic plants and/or plant cells, characterized in that the content and/or the activity of at least one endogenous MLO protein is decreased in comparison to the wild type.

The decrease of the Mlo content and/or activity in a transgenic plant or plant cell according to the invention is preferably at least 10%, 15%, 20% or 25%, more preferably at least 30%, 35%, 40% or 45%, especially preferably at least 50%, 55%, 60% or 65%, particularly preferably at least 70%, 75%, 80% or 85%, and most preferably at least 90%, 92%, 94%, 95%, 96%, 97%, 98%, 99% or 100%.

The decrease of the content and/or the activity of an MLO protein can be achieved by different means. One preferred method is the transfer of at least one nucleic acid molecule comprising at least one sequence which is identical, homologous or complementary to the sequence(s) encoding the endogenous MLO or fragments thereof to the plant cells.

According to the present invention, a “nucleic acid molecule” can be a DNA molecule, e.g. comprising a genomic sequence or a cDNA sequence, or an RNA molecule. The molecule can be double-stranded or single-stranded. Examples of such molecules are double-stranded RNA molecules or vectors, e.g. plasmids, cosmids, recombinant viruses or minichromosomes. The nucleic acid molecule can comprise sequences that derive from the species of the host cell or from another organism/species. Furthermore, those sequences can be natural or modified or synthetic.

The “transfer” of a nucleic acid molecule into a plant or plant cell can be performed by different methods. Preferably, the transfer occurs via transformation, transfection (stable or transient), injection, biolistic methods and/or electroporation, especially when the nucleic acid molecule is DNA. The DNA can e.g. be present in the form of a vector or a linear “promoter-gene-terminator construct” without a common vector backbone. When the molecule s a double stranded RNA, the transfer can be performed by means of biolistic methods. The skilled person is familiar with those methods and will be easily able to identify the best transfer method for his actual requirements. Some of the transfer methods will be described in detail (see below).

A nucleic acid sequence which is “identical” to a sequence encoding an MLO protein (or fragments thereof) is meant to be identical over a certain region, preferably over the whole region of one of the sequences.

A nucleic acid sequence which is “homologous” to a sequence encoding an MLO protein (or fragments thereof) means, in the scope of the present invention, that the sequence is at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% similar to a nucleic acid sequence encoding any of the MLO proteins depicted in SEQ ID NOs: 2, 4, 7, 9, 11, 14, 16, 18, 20, 22, 24, 26, 28, 30, 32, 34, 36, 38, 40, 42, 44, 46 and 48 or their functionally equivalent parts or fragments. Preferably, the homology is determined over the whole sequence length of the nucleic acid molecules. The MLO encoding nucleic acid sequences can be easily deduced from said amino acid sequences by any skilled person. Some of the respective coding nucleic acid sequences are depicted in SEQ ID NOs: 1, 3, 5, 6, 8, 10, 12, 13, 15, 17, 19, 21, 23, 25, 27, 29, 31, 33, 35, 37, 39, 41, 43, 45 and 47. Those sequences are of course not limiting. The skilled person will also adapt the nucleic acid sequences to the preferred codon usage of the host cell.

The following Table 2 gives an overview of the above-mentioned amino acid and nucleic acid sequences:

| | | | nucleic acid/ | | NCBI |
|---|---|---|---|---|---|
| SEQ ID | | | amino | | accession |
| NO: | organism | name | acid | comment | number |
| 1 | Hordeum vulgare | HvMlo | na | wt, full length | |
| 2 | | HvMlo | aa | wt, full length | P93766 |
| 3 | Glycine max | GmMlo1 | na | full length | |
| 4 | | GmMlo1 | aa | full length | |
| 5 | | GmMlo2 | na | genomic, | |
| | | | | partial | |
| 6 | | GmMlo2 | na | EST, partial | |
| 7 | | GmMlo2 | aa | EST | |
| 8 | | GmMlo3.1 | na | full length | |
| 9 | | GmMlo3.1 | aa | full length | |
| 10 | | GmMlo3.2 | na | EST | |
| 11 | | GmMlo3.2 | aa | predicted | |
| 12 | Oryza sativa | OsMlo | na | partial | |
| 13 | | OsMlo | na | genomic | |
| 14 | | OsMlo | aa | | O49914 |
| 15 | Linum | LuMlo | na | | |
| 16 | usitatissimum | LuMlo | aa | | CAA06487 |
| 17 | Triticum aestivum | TaMlo | na | | |
| 18 | | TaMlo | aa | | AAS93630 |
| 19 | Arabidopsis | AtMlo1 | na | | |
| 20 | thaliana | AtMlo1 | aa | | O49621 |
| 21 | | AtMlo2 | na | | |
| 22 | | AtMlo2 | aa | | Q9SXB6 |
| 23 | | AtMlo3 | na | | |
| 24 | | AtMlo3 | aa | | Q94KB9 |
| 25 | | AtMlo4 | na | | |
| 26 | | AtMlo4 | aa | | O23693 |
| 27 | | AtMlo5 | na | | |
| 28 | | AtMlo5 | aa | | O22815 |
| 29 | | AtMlo6 | na | | |
| 30 | | AtMlo6 | aa | | Q94KB7 |
| 31 | | AtMlo7 | na | | |
| 32 | | AtMlo7 | aa | | O22752 |
| 33 | | AtMlo8 | na | | |
| 34 | | AtMlo8 | aa | | O22757 |
| 35 | | AtMlo9 | na | | |
| 36 | | AtMlo9 | aa | | Q94KB4 |
| 37 | | AtMlo10 | na | | |
| 38 | | AtMlo10 | aa | | Q9FKY5 |
| 39 | | AtMlo11 | na | | |
| 40 | | AtMlo11 | aa | | Q9FI00 |
| 41 | | AtMlo12 | na | | |
| 42 | | AtMlo12 | aa | | O80961 |
| 43 | | AtMlo13 | na | | |
| 44 | | AtMlo13 | aa | | Q94KB2 |
| 45 | | AtMlo14 | na | | |
| 46 | | AtMlo14 | aa | | Q94KB1 |
| 47 | | AtMlo15 | na | | |
| 48 | | AtMlo15 | aa | | NP_973686 |

According to one preferred embodiment of the invention, a part of the transferred nucleic acid molecule is at least 50%, more preferably at least 60%, especially preferably at least 70%, particularly preferably at least 80%, also particularly preferably at least 90%, and most preferably at least 95% homologous to the sequence encoding the endogenous MLO or fragments thereof.

A nucleic acid sequence which is “complementary” to a sequence encoding an MLO protein (or fragments thereof) means, in the scope of the present invention, that the sequence can hybridize under stringent conditions with a nucleic acid sequence encoding an MLO protein (or fragments thereof) due to hydrogen bonds between complementary bases. This hybridization has to be specific. The person skilled in the art knows that two sequences do not need to have a 100% complementarity in order to hybridize with one another. Herein, a “complementary” nucleic acid sequence is at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% complementary to a nucleic acid sequence encoding any of the MLO proteins depicted in SEQ ID NOs: 2, 4, 7, 9, 11, 14, 16, 18, 20, 22, 24, 26, 28, 30, 32, 34, 36, 38, 40, 42, 44, 46 and 48 or their functionally equivalent parts or fragments.

In the context of this invention the term “hybridization under stringent conditions” means that the hybridization is performed in vitro under conditions stringent enough to ensure a specific hybridization. Stringent in vitro hybridization conditions are known to the person skilled in the art, and can be found in the literature (e.g. Sambrook and Russell (2001) Molecular Cloning: A Laboratory Manual, 3^{rd} edition, Cold Spring Harbour Laboratory Press, Cold Spring Harbour, N.Y.). The term “specific hybridization” refers to the fact that a molecule preferably binds to a certain nucleic acid sequence, the target sequence, under stringent conditions, if the target sequence is part of a complex mixture of, for example, DNA or RNA molecules, but does not bind, or at least to a considerably lesser degree, to other sequences.

Stringent conditions depend on the conditions. Longer sequences hybridize specifically at higher temperatures. In general, stringent conditions are selected so that the hybridization temperature is approximately 5° C. below the melting point (Tₘ) for the specific sequence at a defined ionic strength and a defined pH value. Tₘ is the temperature (at a defined pH value, a defined ionic strength and a defined nucleic acid concentration) at which 50% of the molecules complementary to the target sequence hybridize to the target sequence in the equilibrium state. Typically, stringent conditions are those in which the salt concentration is at least about 0.01 to 1.0 M of sodium ion concentration (or the concentration of another salt) at a pH of between 7.0 and 8.3 and the temperature is at least 30° C. for short molecules (i.e. for example 10 to 50 nucleotides). Furthermore, stringent conditions can comprise the addition of agents, such as formamide, which destabilizes the hybrids. A preferred, non-limiting example for stringent hybridization conditions are hybridizations in 6× sodium chloride/sodium citrate (SSC) at about 45° C., followed by one or more washing steps in 0.2×SSC, 0.1% SDS at 50 to 65° C. The temperature ranges, for example, under standard hybridization conditions depending on the type of nucleic acid, between 42° C. and 58° C. in an aqueous buffer at a concentration of 0.1 to 5×SSC (pH 7.2).

If an organic solvent, e.g. 50% formamide, is present in the above-mentioned buffer, the temperature under standard conditions is about 42° C. Preferably, the hybridization conditions for DNA:DNA hybrids are for example 0.1×SSC and 20° C. to 45° C., preferably 30° C. to 45° C. Preferably, the hybridization conditions for DNA:RNA hybrids are for example 0.1×SSC and 30° C. to 55° C., preferably between 45° C. to 55° C. The hybridization temperatures mentioned above are determined for example for a nucleic acid having a length of about 100 base pairs and a G/C content of 50% in the absence of formamide. The person skilled in the art knows how the required hybridization conditions can be determined using the above mentioned, or the following, textbooks: Current Protocols in Molecular Biology, John Wiley & Sons, N.Y. (1989), Hames and Higgins (publisher) 1985, Nucleic Acids Hybridization: A Practical Approach, IRL Press at Oxford University Press, Oxford; Brown (publisher) 1991, Essential Molecular Biology: A Practical Approach, IRL Press at Oxford University Press, Oxford.

Typical hybridization and wash buffers have, e.g. the following composition (this example is not limiting):

Prehybridization solution:
0.5% SDS
5×SSC
50 mM NaPO₄, pH 6.8
0.1% Na pyrophosphate
5×Denhardt's solution
100 μg/ml salmon sperm
   Hybridisation solution:
Prehybridization solution
1×10⁶ cpm/ml probe (5-10 min 95° C.)

### 20×SSC:

3 M NaCl
0.3 M sodium citrate
ad pH 7 with HCl
   50×Denhardt's reagent:
5 g Ficoll
5 g polyvinyl pyrrolidone
5 g bovine serum albumine
ad 500 ml A. dest.

A typical method for hybridization is as follows (this example is not limiting):

| Optional: | wash blot 30 min in 1 x SSC/0.1% SDS at 65° C. | | |
|---|---|---|---|
| Prehybridization: | at least 2 hrs at 50-55° C. | | |
| Hybridisation: | over night at 55-60° C. | | |
| Wash: | 05 min | 2x SSC/0.1% SDS | hybridization temp. |
| | 30 min | 2x SSC/0.1% SDS | hybridization temp. |
| | 30 min | 1x SSC/0.1% SDS | hybridization temp. |
| | 45 min | 0.2x SSC/0.1% SDS | 65° C. |
| | 5 min | 0.1x SSC | room temperature |

The person skilled in the art knows that the specified solutions and the protocol shown can or must be modified dependent upon the application.

According to one preferred embodiment of the invention, a part of the transferred nucleic acid molecule is at least 50%, preferably at least 60%, more preferably at least 70%, especially preferably at least 80%, particularly preferably at least 90%, and most preferably at least 95% complementary to the sequence encoding the endogenous MLO or fragments thereof.

According to another preferred embodiment of the present invention, the part of the transferred nucleic acid molecule which is identical, homologous or complementary to the sequences encoding the endogenous MLO or fragments thereof comprises 20 to 1000 nucleotides, preferably 20 to 750 nucleotides, more preferably 20 to 500 nucleotides, especially preferably 20 to 250 nucleotides, particularly preferably 20 to 150 nucleotides, also particularly preferably 20 to 100 nucleotides and most preferably about 20 to 50 nucleotides.

The decrease of the content and/or the activity of the at least one endogenous MLO can be achieved by different methods, e.g. by RNA interference (RNAi), an antisense construct, a co-suppression construct, post-transcriptional gene silencing (PTGS), a ribonuclease P construct, homologous recombination, a ribozyme construct or virus induced gene silencing (VIGS). The methods will be explained in the following.

An increased resistance against soybean rust in transgenic plants or plant cells having a decreased MLO content/activity can be achieved, e.g., by the process of “silencing”. During this process, a nucleic acid which encodes at least one MLO or fragments thereof and/or a nucleic acid which is complementary thereto is transferred to a plant cell. In order to ensure that the plant cell is transgenic for the transferred nucleic acid, usually the nucleic acid to be transferred is part of a vector, e.g. a plasmid, which is able to stably replicate within the cell or which assures the integration of the transferred nucleic acid into the plant genome.

Preferably the silencing of mlo is realized by means of the RNAi method. In this method, a vector is transferred to a plant cell which comprises the following elements in 5′-3′ orientation: a promoter sequence which is functionally active in plants, operatively linked thereto an antisense sequence which is complementary to the sequence encoding the at least one MLO or fragments thereof (or a homologous of this antisense sequence), wherein the sequence has 3′ exon sequences at its 3′ end which are recognizable by the spliceosome, operatively linked thereto an intron, operatively linked thereto a sense sequence which is identical or homologous to the sequence encoding the at least one MLO or fragments thereof, wherein the sequence has 5′ exon sequences at its 5′ end which are recognizable by the spliceosome, and optionally, operatively linked thereto a termination sequence which is functionally active in plants. Of course, the position of the sense and the antisense sequence can be interchanged. It is obvious to the skilled person that in this case, the respective 3′ splicing site and 5′ splicing site need to be adapted.

When those vectors are stably transferred to plant cells, the transcription leads to the generation of a pre-mRNA containing a first exon comprising the antisense sequence, an intron, and a second exon comprising the sense sequence. The intron is then removed via the splicing process, which results in a continuous RNA molecule having regions which are complementary to each other. This molecule will develop a double stranded structure (Smith et al., 2000, Nature, 407:319-320).

Those double stranded RNS molecules are able to silence specifically the mlo mRNA via induction of the PTGS (post transcriptional gene silencing) system. As a consequence, the MLO protein cannot be expressed anymore. The choice of the antisense and sense sequences allows to determine which kind of mlo should be suppressed. The skilled person is able to identify the sequences which are characteristic for the protein. He further knows that a multitude of MLO proteins can be silenced when many corresponding characteristic sequences are used.

This RNAi method can comprise the following steps:
a) Construction of a vector comprising the following nucleic acid sequences in 5′-3′ orientation:
   a promoter sequence which is functionally active in plants,
   operatively linked thereto an antisense sequence which is complementary to the sequence encoding the at least one MLO or fragments thereof, or a homologous of this antisense sequence, wherein the sequence has 3′ exon sequences at its 3′ end which are recognizable by the spliceosome,
   operatively linked thereto an intron,
   operatively linked thereto a sense sequence which is identical or homologous to the sequence encoding the at least one MLO or fragments thereof, wherein the sequence has 5′ exon sequences at its 5′ end which are recognizable by the spliceosome,
   optionally, operatively linked thereto a termination sequence which is functionally active in plants,
b) transfer of the vector from step a) to a plant cell and optionally integration into the plant genome.

The skilled person knows which vectors to choose for the implementation of the RNAi method or the PTGS method. Those vectors can e.g. be constructed in a way to allow the sense and antisense sequences to be transcribed from any appropriate promoter, to hybridize within the cell and to induce the PTGS system (Tuschl, 2002, Nat. Biotechnol. 20, 446-448; Miyagishi et al., 2002, Nat. Biotechnol., 20, 497-500; Lee et al., 2002, Nat. Biotechnol., 20, 500-505). Other vectors combine the sense and antisense sequence by means of a “loop” sequence and are transcribed from any appropriate promoter. The back-folding of the loop allows the sense and antisense sequence to hybridize, to form double stranded RNA and to induce the PTGS system (Tuschl, 2002, vide supra; Paul et al., 2002, Nat. Biotechnol., 20, 505-508; Paddison P. J. Genes Dev. 2002 Apr. 15; 16(8):948-58; Brummelkamp et al., 2002, Science, 296, 550-553).

In another RNAi method, pre-synthesized double stranded RNA molecules comprising the above-mentioned sense and antisense sequences are transferred directly into the plant cells, e.g. by means of biolistic methods. Accordingly, this RNAi method can comprise the following steps:
a) Construction of a double stranded RNA molecule having a length of 15 to 100 nucleotides, preferably of 20 to 75 nucleotides, more preferably of 20 to 50 nucleotides, especially preferably of 20 to 40 nucleotides, particularly preferably of 20 to 30 nucleotides and most preferably of 20 to 25 or 21, 22 or 23 nucleotides, comprising a nucleic acid sequence having a sense strand which is identical or homologous to a fragment of the sequence(s) encoding the at least one endogenous MLO,
b) transfer of the molecule from step a) to a plant cell.

In another preferred embodiment, the vectors which are used for the transfer of nucleic acids comprise, in 5′-3′ orientation: a promoter sequence, a sense sequence which is identical or homologous to the sequence encoding the at least one endogenous MLO or fragments thereof, wherein the sequence has self-complementary regions, and optionally a termination sequence. The transcription of those vectors in the plant cell results in the generation of RNA molecules which contain sequence regions being able to hybridize with themselves. This can lead to the formation of double stranded RNA molecules inside of the cell, which can induce the PTGS system and which results in the specific degradation of mlo mRNA. This method for the silencing of plant proteins, also called co-suppression, requires that the mRNA of the MLO to be suppressed contains regions which are complementary to each other. Such regions can be identified by the skilled person by a visual inspection of the respective coding DNA sequence or by means of sequence programs like DNAStar from DNAStar Inc., Madison, USA.

This co-suppression method can comprise the following steps:
a) Construction of a vector comprising the following nucleic acid sequences in 5′-3′ orientation:
   a promoter sequence which is functionally active in plants,
   operatively linked thereto a sense sequence which is identical or homologous to the sequence encoding the at least one endogenous MLO or fragments thereof, wherein the sequence has self-complementary regions,
   optionally, operatively linked thereto a termination sequence which is functionally active in plants,
b) transfer of the vector from step a) to a plant cell and optionally integration into the plant genome.

In another preferred embodiment of the present invention, the vectors which are used for the transfer of the nucleic acids comprise, in 5′-3′ orientation: a promoter sequence, operatively linked thereto an antisense sequence which is complementary to the sequence encoding the at least one endogenous MLO or fragments thereof (or a homologous of this antisense sequence), and optionally a termination sequence. The transcription of those vectors in plant cells results in the generation of an RNA molecule, the sequence of which is complementary to the mRNA encoding an MLO or parts thereof. Hybridization of the antisense sequence with the endogenous mRNA sequences of mlo in vivo can then lead to the suppression of the mlo expression in plant cells.

This antisense method can comprise the following steps:
a) construction of a vector comprising the following nucleic acid sequences in 5′-3′ orientation:
   a promoter sequence which is functionally active in plants,
   operatively linked thereto an antisense sequence which is complementary to the sequence encoding the at least one endogenous MLO or fragments thereof, or a homologous of this antisense sequence,
   optionally, operatively linked thereto a termination sequence which is functionally active in plants,
b) transfer of the vector from step a) to a plant cell and optionally integration into the plant genome.

In another preferred embodiment of the present invention, the vectors which are used for the transfer of the nucleic acids comprise in 5′-3′ orientation: a promoter sequence, operatively linked thereto a DNA sequence encoding a ribozyme which specifically recognizes the mRNA of the at least one mlo, and optionally a termination sequence. It is well known to the skilled person how to produce ribozymes which have an endonuclease activity which is directed against a specific mRNA. In detail, this method is e.g. described in Steinecke P et al. (EMBO J. 1992 April; 11(4):1525-30). In the context of the present invention, the term “ribozyme” also comprises those RNA sequences which include in addition to the ribozyme itself leader sequences which are complementary to the mRNA of the mlo or fragments thereof and which are therefore able to guide the mRNA specific ribozyme even more efficiently to the mRNA substrate.

This ribozyme method can comprise the following steps:
a) construction of a vector comprising the following nucleic acid sequences in 5′-3′ orientation:
   a promoter sequence which is functionally active in plants,
   operatively linked thereto a DNA sequence encoding a ribozyme which specifically recognizes the mRNA of the at least one endogenous mlo,
   optionally, operatively linked thereto a termination sequence which is functionally active in plants,
b) transfer of the vector from step a) to a plant cell and optionally integration into the plant genome.

Another alternative for increasing the resistance against soybean rust in transgenic plants or plant cells is the transfer of nucleic acids via vectors which comprise in 5′-3′ orientation: a promoter sequence, operatively linked thereto a DNA antisense sequence which is complementary to the sequence encoding the mRNA of the at least one mlo or fragments thereof, operatively linked thereto a sequence encoding a ribonuclease P (RNAse P), and optionally, operatively liked thereto a termination sequence. The transcription of these vectors in the cell results in RNA molecules which include a leading sequence (the antisense sequence), which guides the RNAse P to the mlo mRNA, whereupon the degradation of the mRNA by the RNAse P occurs (see U.S. Pat. No. 5,168,053). Preferably, the leading sequence comprises 10 to 15 nucleotides which are complementary to the DNA sequence of the mlo, and one 3′-NCCA nucleotide sequence, wherein the N is preferably a purine. The transcripts of the external leading sequence bind to the target mRNA via formation of base pairs, which allows the degradation of the mRNA by the RNAse P at the 5′ nucleotide of the paired region. This degraded mRNA cannot be translated into a functional protein.

This RNAse P method can comprise the following steps:
a) construction of a vector comprising the following nucleic acid sequences in 5′-3′ orientation:
   a promoter sequence which is functionally active in plants,
   operatively linked thereto a DNA sequence which is complementary to the sequence encoding the mRNA of the at least one MLO or fragments thereof,
   operatively linked thereto a sequence encoding a ribonuclease P,
   optionally, operatively linked thereto a termination sequence which is functionally active in plants,
b) transfer of the vector from step a) to a plant cell and optionally integration into the plant genome.

Furthermore, vectors can be used for the method according to the invention, which comprise the following sequence in 5′-3′ orientation: a DNA sequence which is identical or homologous to the sequence encoding the 5′ end of the at least one endogenous MLO, a promoter sequence, operatively linked thereto a DNA sequence encoding a resistance or reporter gene, optionally a termination sequence, and a DNA sequence which is identical or homologous to the sequence encoding the 3′ end of the at least one endogenous MLO. Those vectors can be used in order to induce a specific knock-out of the mlo of interest by means of homologous recombination. The sequence of the resistance or reporter gene is inserted in those plant cells in which the homologous recombination has occurred, so that no functional mlo mRNA can be produced in the cell. The plant cells in which the recombination has occurred can be identified by selection of the resistance or reporter gene. The skilled person knows how to produce those vectors for genetic knock-out via homologous recombination, which elements they have to comprise (promoters, enhancers, flanking sequences) and how to identify the respective plant cells. Usually, antibiotic resistance genes are used as resistance genes (Amp, Kan etc.). Of course, all other possible resistance genes can be used which allow the selection of the cells in which the recombination has occurred. In addition to the classical resistance genes, other reporter genes can be used for the detection and/or selection of the plants and plant cells in which the homologous recombination has occurred, such as GUS, GFP etc.

This homologous recombination method can comprise the following steps:
a) construction of a vector comprising the following nucleic acid sequences in 5′-3′ orientation:
   a DNA sequence which is identical or homologous to the sequence encoding the 5′ end of the at least one endogenous MLO,
   a promoter sequence which is functionally active in plants,
   operatively linked thereto a DNA sequence encoding a resistance or reporter gene,
   optionally, operatively linked thereto a termination sequence which is functionally active in plants,
   a DNA sequence which is identical or homologous to the sequence encoding the 3′ end of the at least one endogenous MLO,
b) transfer of the vector from step a) to a plant cell and optionally integration into the plant genome.

According to the present invention, nucleic acid sequences encoding an MLO or fragments thereof can be the complete coding DNA sequence for MLO or the complete mRNA sequence or fragments thereof. Since some of the above-mentioned methods for the production of transgenic plants, being directed to a significant reduction of mlo expression, are based on a specific hybridization of an endogenous mlo mRNA and the sequences which are generated during the transcription of the above-mentioned vectors (e.g. the antisense strategy), the skilled person knows that the transferred nucleic acids do not necessarily have to contain the complete sequence encoding the MLO, irrespective if it is a sense or an antisense sequence. In fact, relatively short regions of the sequences encoding the MLO are sufficient for a specific hybridization and an efficient silencing.

Those sequences of the vectors which correspond to the sequence regions of the mlo mRNA and which are transcribed to generate double stranded RNA molecules can have a length of about 25 nucleotides, preferably 21, 22 or 23 nucleotides. The sequences which are transferred for the antisense strategy usually comprise between 20 and 1000 nucleotides, preferably between 20 and 800 nucleotides, more preferably between 400 and 800 nucleotides, especially preferably between 500 and 750 nucleotides. But it is also possible to use sequences comprising between 20 and 500, between 20 and 300, between 20 and 150, between 20 and 100 or between 20 and 50 nucleotides. The skilled person knows that for the RNAi or the PTGS method, the sense and antisense RNAs which are used for the generation of double stranded RNA molecules can also comprise about 21, 22 or 23 nucleotides with a characteristic 3′ overhang (Tuschl, 2002, Nat. Biotechnol. 20, 446-448).

When nucleic acids are transferred to plant cells, and the transcription of those sequences in the cell results in sequences which are complementary to the mlo mRNA (e.g. using the antisense strategy), those transferred sequences do not need to be 100% complementary to the mRNA. It will be sufficient if the sequences are at least 50%, preferably at least 60%, more preferably at least 70%, especially preferably at least 80%, particularly at least 90% and most preferably at least 95% complementary. The differences can be the result of insertions, deletions and/or substitutions, preferably substitutions. The skilled person knows however that with decreasing complementarity, the probability to silence several mlo mRNAs will increase.

In general, only those complementary sequences can be used for the present invention which are able to specifically hybridize with regions of the mlo mRNA. Sequences which hybridize in vivo with RNA regions of proteins other than MLO and which cause their silencing are not adequate for the present invention. Depending on the selected sequence and on the degree of complementarity, a multitude of MLO proteins or only a few MLO proteins will be silenced. It is also possible that the expression of only one specific mlo is inhibited. The length of complementary sequences is preferably between 20 and 1000 nucleotides, more preferably between 20 and 750 nucleotides, especially between 20 and 500 nucleotides, particularly preferably between 20 and 300 nucleotides and most preferably between 20 and 150, 20 and 75 or 20 and 50 nucleotides. It is also possible that the sequences only comprise about 20 or 25 nucleotides.

Some of the above-mentioned methods can also be performed with sequences which are not part of the coding region of the mlo mRNA or which are not complementary thereto. It can e.g. be sufficient that those sequences derive from the 5′ or 3′ untranslated region, if those regulatory sequences are characteristic for the mRNA of the respective MLO. Those sequences can be used especially when the silencing is induced via double stranded RNA constructs or when the translation of an mRNA is inhibited by antisense constructs. Therefore, in the context of the invention, the term “mRNA” not only comprises coding regions, but also regulatory regions which occur in the pre-mRNA or in the mature mRNA and which are characteristic for the mlo mRNA. The same applies for the DNA sequence, e.g. for untranscribed sequences, promoter sequences, upstream activating sequences, introns etc.

If vectors are used whose transcription results in the generation of RNA molecules which have a leading sequence and an RNAse P, the leading sequence has to be sufficiently complementary in order to specifically recognize the mlo mRNA. The conditions allow the skilled person to choose which part of the mlo mRNA is recognized by the leading sequence. Preferably the leading sequences comprise about 20 nucleotides, they should however not be shorter than about 15 nucleotides. Having a 100% complementarity of the leading sequence, 12 nucleotides can be sufficient. Of course, the leading sequence can comprise up to about 100 nucleotides or even more, because this will increase the specificity for the respective mRNA.

In the context of the present invention, “sense sequences” (or sense strands) are those sequences which correspond to the coding strand of the mlo gene(s) or fragments thereof. Those sequences do not necessarily need to be 100% identical with the sequences encoding the Mlo of interest. It will be sufficient that the sequences are similar (homologous) enough to the sequences encoding the MLO(s) that their expression in plant cells results in an efficient and specific silencing of the mlo(s), e.g. via RNA interference or co-suppression. It will be sufficient if those sequences are at least 50%, preferably at least 60%, more preferably at least 70%, especially preferably at least 80%, particularly at least 90% and most preferably at least 95% homologous. The differences can be the result of insertions, deletions, additions and/or substitutions. When sequences have those degrees of identity, they are usually called to be homologous (see above). The skilled person knows however that with decreasing identity or homology, the probability to silence several mlo mRNAs will increase. Sequences having a very low degree of similarity or homology, i.e. sequences that will also silence other proteins than MLO, are not sufficiently specific and therefore not suitable for the present invention.

Accordingly, “antisense sequences” (or antisense strands) are those sequences which correspond to the non-coding DNA strand of the genes of the MLO of interest. Those sequences do not necessarily need to be 100% identical with the sequence of the non-coding DNA strands of the genes of interest, but can have the above mentioned degrees of homology. For example, the antisense sequence can be at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% homologous to the non-coding mlo strand (or complementary to the coding strand). As mentioned above, it is sufficient that those antisense sequences are able to hybridize specifically with the respective mlo mRNA. The hybridization can take place either in vivo under cellular conditions or in vitro. The hybridization of an antisense sequence with an endogenous mRNA sequence usually takes place in vivo under cellular conditions.

The terms “sense” and “antisense” are well known to the person skilled in the art. The person skilled in the art of silencing genes in plants knows from the literature how long the nucleic acid molecules, which are used for silencing, have to be and what degree of homology or complementarity they have to exhibit in relation to the sequences of interest. In the context of the present invention, an antisense sequence which does not specifically hybridize with the coding sense sequences of mlo, i.e. which also hybridize with the coding sense sequences of other proteins, cannot be used.

The antisense strategy can be coupled with a ribozyme method. Ribozymes are catalytically active RNA sequences which, being coupled to the antisense sequences, catalytically cleave their target sequences (Tanner et al., (1999) FEMS Microbiol Rev. 23 (3), 257-75). This can increase the efficiency of an antisense strategy.

Other methods of reducing the expression of mlo particularly in plants comprise the over-expression of mlo nucleic acid sequences or their homologues, resulting in co-suppression (Jorgensen et al., (1996) Plant Mol. Biol. 31 (5), 957-973) or the induction of the specific RNA degradation by means of a viral expression system (amplicon) (Angell et al., (1999) Plant J. 20 (3), 357-362). Those methods are also referred to as PTGS (see above).

Other methods are the introduction of nonsense mutations in the endogenous gene via transfer of RNA/DNA oligonucleotides into the plant (Zhu et al., (2000) Nat. Biotechnol. 18 (5), 555-558) or the generation of knockout mutants by means of T-DNA mutagenesis (Koncz et al., (1992) Plant Mol. Biol. 20 (5) 963-976) or homologous recombination (Hohn et al., (1999) Proc. Natl. Acad. Sci. USA. 96, 8321-8323).

Furthermore a gene repression (but also the gene overexpression) can also be performed by means of specific DNA binding factors, e.g. factors of the type of zinc finger transcription factors. Also, factors can be introduced into a cell which inhibit the target protein. The protein binding factors can be for example aptamers (Famulok et al., (1999) Curr Top Microbiol Immunol. 243, 123-36). They are expressed via vector-based overexpression, and their design and selection can be easily performed by the skilled person.

An overview over the above-mentioned methods can be found, e.g., in Waterhouse et al., (2001), Nature 411, 834-842; Tuschl (2002), Nat. Biotechnol. 20, 446-448; Paddison et al., (2002), Genes Dev., 16, 948-958; Brummelkamp et al., (2002), Science 296, 550-553.

Another aspect of the present invention is a method of increasing resistance against soybean rust in transgenic plants and/or plant cells, wherein the content and/or the activity of at least one endogenous MLO is decreased by the expression of at least one non-functional MLO or a fragment thereof which has at least one point mutation, deletion and/or insertion. The non-functional MLO proteins have lost completely or to a very important degree their capacity to interact with the common physiological or pathogenic binding partners. Those non-functional mutants can comprise one or more amino acid insertions, deletions or point mutations. They are useful for the production of transgenic plants or plant cells in which the content of endogenous MLO protein is not altered, but the activity of the endogenous MLO is blocked by means of the overexpression of said non-functional MLO mutants. Furthermore, those resistant plants have the advantage to exhibit an essentially normal phenotype.

Non-functional MLO proteins or mutants have essentially the same nucleic acid and amino acid sequences as their functional counterparts. However, they comprise one or more insertions, deletions or point mutations of nucleotides or amino acids, which cause a dramatic decrease of the capacity of the mutated MLO protein to interact with its binding partners. The skilled person has a series of methods at hand which allow him to insert point mutations, deletions or insertions into the nucleic acid sequences encoding the functional or non-functional MLO proteins (Sambrook (2001), Molecular Cloning: A Laboratory Manual, 3rd edition, Coldspring Harbour Laboratory Press; “PCR technology: Principle and Applications for DNA Amplification”, H. Ehrlich, id, Stockton Press).

The reduced binding efficiency of those MLO mutants to the physiological and/or pathogenic binding partners in comparison to the wild type (non mutated) MLO proteins is preferably in the range of over 1% to 90%, more preferably over 1% to 70%, especially preferably over 1% to 50%, particularly preferably over 1% to 30%, and most preferably over 1% to 10%.

Although the non-functional MLO mutants show one or more point mutations, deletions and/or insertions, the term “non-functional” MLO (also called inactive MLO) does not comprise proteins which have no essential sequence homology to the functional MLO proteins as depicted in SEQ ID NOs: 2, 4, 7, 9, 11, 14, 16, 18, 20, 22, 24, 26, 28, 30, 32, 34, 36, 38, 40, 42, 44, 46 and 48.

Preferably, the at least one point mutation, deletion and/or insertion of the non-functional MLO prevent the cellular function of MLO, and especially inhibit the interaction of MLO with its pathogenic or physiologic binding partners, especially with ROR2 and/or calmodulin.

The non-functional MLO mutant being expressed or overexpressed in the transgenic plants according to the invention does not necessarily have to be the same MLO than the endogenous MLO of the host cell, but can also derive from another organism/species. The important characteristic of the non-functional MLO mutant is their competition with the activity of the endogenous MLO. Of course, a high degree of sequence homology between those two proteins will favor a high competitive activity of the non-functional MLO.

According to a preferred embodiment of the present invention, the non-functional MLO is a dominant negative MLO. The “dominant negative method” can comprise the following steps:
a) Construction of a vector comprising the following nucleic acid sequences in 5′-3′ orientation:
   a promoter sequence which is functionally active in plants,
   operatively linked thereto a DNA sequence encoding a dominant negative mutant of the at least one endogenous Mlo,
   optionally, operatively linked thereto a termination sequence which is functionally active in plants,
b) transfer of the vector from step a) to a plant cell and optionally integration into the plant genome.

The person skilled in the art can identify dominant negative mutants by means of routine methods. He can, e.g., introduce mutations into a wild type MLO sequence and perform in vitro binding assays of the obtained mutants with binding partners such as ROR2 or calmodulin. In the same way, the skilled person can test whether the MLO non-functional mutants compete with their wild type counterparts in terms of interactions with known binding partners.

A “dominant negative mutant”, in the scope of the present invention, is every mutant (insertion, deletion, point mutation) which is capable of inhibiting the interaction of an MLO with its pathogenic or physiologic binding partners, such as ROR2 and/or calmodulin.

Transgenic plants or plant cells having an increased resistance against soybean rust can also be produced by a method which is characterized in that the content and/or the activity of at least one endogenous Mlo is decreased by the expression of at least one recombinant antibody which is specific for at least one endogenous Mlo and which prevents the cellular function of the Mlo, and which especially inhibits the interaction of the Mlo with its pathogenic or physiologic binding partners, especially with Ror2 and/or calmodulin.

The skilled person knows from the literature how those antibodies, which are e.g. specific for a certain MLO domain, can be produced, isolated and identified. According to the present invention, the term “recombinant antibody” comprises all of the different forms and types of antibodies, such as described in Skerra A. (Curr Opin Immunol. 1993 April; 5(2):256-62). Examples are Fab fragments, Fv fragments, scFv antibodies, scFv homodimers, VH chains etc. A review is given by Conrad U. and Fiedler U. (Plant Mol. Biol. 1998 September; 38(1-2):101-9). Standard protocols for the production of monoclonal, polyclonal or recombinant antibodies can be found in: “Guide to Protein Purification”, Meth. Enzymol. 182, pp. 663-679 (1990), M. P. Deutscher, ed. The expression of antibodies is also described in Fiedler et al., (1997) Immunotechnology 3, 205-216 and Maynard and Georgiou (2000) Annu. Rev. Biomed. Eng. 2, 339-76.

Preferred in the present invention are scFv antibodies which consist of the variable region of a light chain and the variable region of a heavy chain, being fused with one another by a flexible linker peptide (see, e.g. Breitling et al. (1999) Recombinant Antibodies, John Wiley & Sons, New York). ScFv antibodies have the same antigen specificity and activity as “normal” antibodies, but do not need to be assembled from single chains.

Usually, the production of a recombinant antibody starts with hybridoma cell lines expressing monoclonal antibodies. The cDNAs encoding the light and the heavy chain are isolated, and in a next step the coding regions for the variable regions of the light and the heavy chain are fused to one molecule. Another method of obtaining recombinant antibodies is based on the screening of recombinant antibody libraries, so-called phage display libraries (see Hoogenboom et al. (2000) Immunology Today 21, 371-378; Winter et al. (1994) Annu. Rev. Immunol. 12, 433-455; De Wildt et al. (2000) Nat. Biotechnol. 18, 989-994). This method allows the enrichment, selection and isolation of the desired antibody against an MLO protein.

One method of expression of an anti-MLO antibody can comprise the following steps:
a) construction of a vector comprising the following nucleic acid sequences in 5′-3′ orientation:
   a promoter sequence which is functionally active in plants,
   operatively linked thereto a DNA sequence encoding a recombinant antibody which is specific for the at least one endogenous Mlo and which prevents the cellular function of Mlo,
   optionally, operatively linked thereto a termination sequence which is functionally active in plants,
b) transfer of the vector from step a) to a plant cell and optionally integration into the plant genome.

Transgenic plants or plant cells having an increased resistance against soybean rust can also be produced by a method which is characterized in that the content and/or the activity of at least one endogenous MLO is decreased by the expression of at least one MLO inhibitor which prevents the cellular function of at least one MLO, and which especially inhibits the interaction of the MLO with its pathogenic or physiologic binding partners, especially with ROR2 and/or calmodulin. These inhibitors can be e.g. peptides which bind in the respective binding pockets of the MLO proteins for the interaction with the physiologic binding components or factors.

This method resembles to the antibody strategy, in that an inhibitor of an MLO protein, being expressed or overexpressed in the plant cell, will block the MLO activity (e.g. sterically) by binding to the MLO. One method of expression of an MLO inhibitor can comprise the following steps:
a) construction of a vector comprising the following nucleic acid sequences in 5′-3′ orientation:
   a promoter sequence which is functionally active in plants,
   operatively linked thereto a DNA sequence encoding an MLO inhibitor which prevents the cellular function of MLO,
   optionally, operatively linked thereto a termination sequence which is functionally active in plants,
b) transfer of the vector from step a) to a plant cell and optionally integration into the plant genome.

In addition to the transfer of a nucleic acid molecule, other methods can be used for increasing the resistance of against soybean rust in transgenic plants or plant cells which have a decreased content and/or activity of at least one endogenous MLO. For example, the MLO content and/or activity can be decreased by mutagenesis, preferably by chemical mutagenesis or radiation induced mutagenesis. The mutagenesis can, e.g. be performed by means of ethyl methane sulfonate (EMS), gamma irradiation and/or fast neutron irradiation.

Induced mutations can also be caused by other chemicals like Nitrosoguanidine (NTG), base analogues (e.g. BrdU), simple chemicals (e.g. acids), alkylating agents (e.g. N-ethyl-N-nitrosourea, ENU), methylating agents (EMS), polycyclic hydrocarbons (e.g. benzpyrenes), DNA intercalating agents (e.g. ethidium bromide), DNA crosslinkers (e.g. platinum), oxygen radicals, or by UV irradiation (non-ionizing) or ionizing radiation. Alkylating agents can mutate both replicating and non-replicating DNA. In contrast, a base analog can only mutate the DNA when the analog is incorporated in replicating the DNA. Each of these classes of chemical mutagens has certain effects that then lead to transitions, transversions, or deletions. UV radiation excites electrons to a higher energy level. DNA absorbs one form, ultraviolet light. Two nucleotide bases in DNA—cytosine and thymine—are most vulnerable to excitation that can change base-pairing properties. UV light can induce adjacent thymine bases in a DNA strand to pair with each other, as a bulky dimer.

The mlo knockout in barley was found by the inventors to confer increased resistance against soybean rust. However, it is conceivable that also a positive resistance effect occurs via MLO. This means that an MLO overexpression, the MLO being derived e.g. from barley or Arabidopsis, could also lead to an increased resistance, especially of soybean. There are examples of proteins which confer resistance in a non-host interaction (as in the present case: barley—soybean rust), whereas in the case of a host interaction (soybean—soybean rust), those proteins confer susceptibility. For example, in susceptible interactions most fungal Avr proteins are pathogenicity factors, but in host-resistant plants the Avr protein is recognized by plant R-proteins. That means Avr proteins have different “functions” in resistant and compatible interactions.

Hence, an MLO overexpression could also confer resistance in soybean. Therefore, another aspect of the present invention is directed to a method of increasing resistance against soybean rust in transgenic plants and/or plant cells, characterized in that the content and/or the activity of the at least one MLO is increased in comparison to the wild type.

The increase is preferably at least 10% or 20%, also preferably at least 30% or 40%, more preferably at least 50% or 60%, also more preferably at least 70% or 80%, especially preferably at least 90%, 95% or 100%, particularly preferably at least by a factor of 2 or 5, also particularly preferably at least by a factor of 10 or 50, and most preferably at least by a factor of 100 or 1000.

In a preferred embodiment of the invention, this increase of the content and/or the activity of at least one MLO in comparison to the wild type can be performed by the transfer of at least one nucleic acid molecule encoding at least one MLO and/or a functionally equivalent fragment thereof and/or a functionally equivalent derivative thereof to the plants or plant cells.

In principle, the nucleic acid molecule can encode any known MLO from any organism (as well as functionally equivalent fragments and/or derivatives thereof). In case that the mlo sequence is of genomic origin from a eukaryotic cell and comprises introns, and in case that the host plant or plant cell is not able or cannot be enabled to splice those introns, it is preferable to use the corresponding cDNA sequence.

The following definitions of the terms “functionally equivalent fragment” and “functionally equivalent derivative” refer to the method of increasing the content and/or the activity of at least one MLO in comparison to the wild type. Therefore the fragments and mutants have to be functional, in contrast to the method of decreasing the content and/or the activity of at least one MLO, where the MLO fragments or mutants can also be non-functional.

In the context of the present invention, a nucleic acid molecule encoding a “functionally equivalent fragment” of an MLO is a fragment or part of a nucleic acid which encodes an MLO protein, having for example an amino acid sequence as depicted in SEQ ID NOs: 2, 4, 7, 9, 11, 14, 16, 18, 20, 22, 24, 26, 28, 30, 32, 34, 36, 38, 40, 42, 44, 46 and 48. The MLO fragment encoded by this nucleic acid has the same cellular functions, the same binding properties and/or the same structural properties as any of the MLO proteins having an amino acid sequence as depicted in SEQ ID NOs: 2, 4, 7, 9, 11, 14, 16, 18, 20, 22, 24, 26, 28, 30, 32, 34, 36, 38, 40, 42, 44, 46 and 48 (see above, definition of “functionally equivalent”). Usually, the fragment lacks amino acids at the N-terminus and/or at the C-terminus.

Preferably, the fragment has at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% of the length of the “whole” MLO protein.

In the context of the present invention, a nucleic acid molecule encoding a “functionally equivalent derivative” of an MLO protein is a derivative or “homologous” or “mutant” of a nucleic acid which encodes an MLO protein, wherein the MLO protein has e.g. an amino acid sequence as depicted in SEQ ID NOs: 2, 4, 7, 9, 11, 14, 16, 18, 20, 22, 24, 26, 28, 30, 32, 34, 36, 38, 40, 42, 44, 46 and 48. The MLO derivative encoded by this nucleic acid has the same cellular functions, the same binding properties and/or the same structural properties as any of the MLO proteins having an amino acid sequence as depicted in S SEQ ID NOs: 2, 4, 7, 9, 11, 14, 16, 18, 20, 22, 24, 26, 28, 30, 32, 34, 36, 38, 40, 42, 44, 46 and 48. Usually, the derivative has one or more amino acid exchanges, insertions or deletions which do not lead to altered cellular functions, binding properties and/or structural properties.

Preferably, the derivative is at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% similar of the “whole” MLO protein.

In a preferred embodiment of the invention, a nucleic acid sequence encoding at least one MLO protein and/or a functionally equivalent fragment thereof and/or a functionally equivalent derivative thereof is transferred to a plant or plant cell. This transfer leads to an increase of the expression or the activity of MLO in comparison to the wild type and therefore to an increase of the resistance against soybean rust in the transgenic cells. The use of vectors comprising those nucleic acid sequences as well as promoter and optional termination sequences are well known to the skilled person. Such a method typically comprises the following steps:
a) Construction of a vector comprising the following nucleic acid sequences in 5′-3′ orientation:
   a promoter sequence which is functionally active in plants,
   operatively linked thereto a DNA sequence encoding at least one MLO and/or a functionally equivalent fragment thereof and/or a functionally equivalent derivative thereof,
   optionally, operatively linked thereto a termination sequence which is functionally active in plants,
b) transfer of the vector from step a) to a plant cell and optionally integration into the plant genome.

The skilled person knows how to transfer a vector from step a) to the plant cells and which characteristics the vector needs in order to be integrated into the plant genome. If the MLO content in transgenic plants or plant cells is increased by means of the transfer of a nucleic acid molecule encoding an Mlo from a different organism, it is preferable that the amino acid sequence is re-translated according to the genetic code into a nucleic acid sequence which mainly comprises codons which are preferably used by the host organism due to its “codon usage”. The codon usage can be determined by means of computer based analyses of other genes known from the respective host organism.

The increase of the content and/or the activity of at least one endogenous Mlo can also be performed by influencing the transcription, the translation and/or the posttranslational modifications of the endogenous Mlo. This means for example that the gene expression of the endogenous Mlo is increased or that inhibiting regulatory mechanisms on the level of transcription, translation or proteins (e.g. post-translational modifications) are turned off.

According to the invention, an increase of the gene expression can e.g. be achieved by influencing the promoter sequence of the endogenous Mlo gene. Such a modification, which preferably leads to an enhancement of the endogenous Mlo expression, can be achieved by deletion or insertion of DNA sequences. The modification of the promoter sequence usually leads to a modification of the amount of expressed Mlo and consequently to a modification of the MLO activity which can be determined in a plant cell.

Furthermore, a modified or increased expression of at least one endogenous Mlo gene can be achieved when a regulatory protein which does not occur in the transformed cell or plant interacts with the promoter of the endogenous Mlo gene. Such a regulator can be a chimeric protein containing a DNA binding domain and a transcription activation domain, as described e.g. in WO 96/06166.

Another possibility for increasing the content and/or the activity of an endogenous Mlo is based on the upregulation of transcription factors which are involved in the transcription of endogenous Mlo genes, e.g. by means of overexpression of those transcription factors. Methods of upregulating transcription factors are well known to the skilled person.

An increase of endogenous MLO can also be achieved when the post-transcriptional modifications of Mlo are influenced. For example, the activity of enzymes like kinases or phosphatases which are involved in this process can be regulated by means of procedures like overexpression or “gene silencing”.

Finally the expression of endogenous Mlo can be regulated via the expression of aptamers which specifically bind to the promoter sequences of Mlo. Depending on whether the aptamers bind to stimulating or repressing promoter regions, the content and therefore also the activity of endogenous Mlo is increased.

In a preferred embodiment of the present invention, the vector which is transferred to a plant or plant cell comprises further regulatory and/or functional sequences in addition to the promoter sequence and the optional termination sequence. More preferably, those regulatory and/or functional sequences are sequences which allow a propagation of the vector in bacteria and/or allow a transient and/or permanent replication in plant cells and/or are selected from the group consisting of enhancers, replication signals and selection markers.

The vectors according to the invention can also include other e.g. enhancer elements as regulatory elements. In addition they can contain resistance genes, replication signals and other DNA regions which allow the propagation of the vectors in bacteria such as E. coli. The regulatory elements also include sequences which bring about stabilization of the vectors in the host cells. In particular, these regulatory elements include sequences which allow stable integration of the vector into the plant's host genome or an autonomous replication of the vector in the plant cells. The person skilled in the art is acquainted with this type of regulatory elements.

With the so-called termination sequences one means sequences which ensure that the transcription or the translation is properly terminated. If the transferred nucleic acids are to be translated, they are typically stop codons and corresponding regulatory sequences; if the transferred nucleic acids are only to be transcribed, they are generally poly-A sequences.

Preferably, the vector is selected from the group consisting of plasmids, cosmids, (recombinant) viruses and other current vectors known in the field of gene technology, with which nucleic acid molecules can be transferred to plants or plant cells. The term “vector” also comprises so-called minochromosomes which are linear or circular DNA fragments which contain centromer sequences of the respective plant in addition to the transgene. Minichromosomes are stable in the nucleus and are passed on to the daughter cells during cell division. They are transferred by standard methods of transformation. Most preferably, the vector is selected from the group consisting of pBR322, pUC vectors, M13 mp vectors or vectors being derived from the Ti plasmid or the Ri plasmid of agrobacteria.

In order to prepare the introduction of foreign genes into higher plants or the cells of the same, a large number of cloning vectors are available which contain a replication signal for E. coli and a marker gene for the selection of transformed bacterial cells. Examples of such vectors are pBR322, pUC series, M13 mp series, pACYC184, etc. The required sequence can be introduced into the vector at an appropriate restriction site. The plasmid obtained is used for the transformation of E. coli cells. Transformed E. coli cells are cultivated in an appropriate medium, and finally harvested and lysed. The plasmid is recovered. As an analysis method for characterizing the plasmid DNA obtained, methods such as restriction analyses, gel electrophoreses and other biochemical/molecular biological methods are generally used. Following each manipulation the plasmid DNA can be cleaved and the DNA fragments obtained can be combined with other DNA sequences. Each plasmid DNA sequence can be cloned into the same or other plasmids. Standard cloning methods can be taken from Sambrook et al., 2001 (Molecular cloning: A laboratory manual, 3^{rd} edition, Cold Spring Harbor Laboratory Press).

The nucleic acid sequences to be transferred are preferably under the control of promoters which are functional in plants. In a preferred embodiment of the present invention, the promoter sequences are selected from the group consisting of constitutive promoters, preferably the 35S promoter, the actin promoter or the ubiquitin promoter, tissue specific promoters, preferably the phosphoenolpyruvate promoter or the fructose-1,6-bisphosphatase promoter, leaf specific promoters, epidermis specific promoters, development specific promoters, light specific promoters, lesion specific promoters or pathogen induced promoters, especially fungus induced promoters.

The promoters can be constitutive, induceable, tissue- or development-specific promoters. Moreover, they can also be pathogen-specific promoters. In this way e.g. transgenic plants can be produced which, under normal circumstances, express the MLO proteins, but if attacked by a pathogen, silence the genes for MLO proteins by means of the pathogen-specific promoter in the cells first affected.

Typically, the constitutive 35S promoter will be used as a promoter for vectors. Moreover, other promoters can, of course, be used, which are obtained from different sources, such as plants or plant viruses or fungi, and which are suitable for the expression of genes in plants. The choice of promoter and of other regulatory sequences determines the local and temporal expression pattern and also the silencing of the MLO proteins in transgenic plants.

Besides additional constitutive promoters, such as the actin promoter (McElroy et al., 1990, Plant Cell, 2:163) and the ubiquitin promoter (Binet et al., 1991, Plant Science, 79:87), the tissue-specific promoters of the phosphoenol pyruvate carboxylase from corn (Hudspeth et al., 1989, Plant Mol. Biol., 12:579) or of the fructose 1,6-bisphosphatase from potato (WO 98/18940), which determine the leaf-specific expression, can also be considered. Lesion induced, light induced or pathogen induced (especially fungus induced) promoters, leaf specific, epidermis specific and development-dependent promoters or control sequences can also be used (Xu et al., 1993, Plant Mol. Biol. 22:573; Logemann et al., 1989, Plant Cell, 1:151; Stockhaus et al., 1989, Plant Cell, 1:805; Puente et al., 1996, EMBO J., 15:3732; Gough et al., 1995, Mol. Gen. Genet., 247:323). A summary of useable control sequences can be found, e.g. in Zuo et al., 2000, Curr. Opin. Biotech., 11:146.

Appropriate promoters also include promoters which guarantee an expression solely in photosynthetically active tissues, e.g. the ST-LS1 promoter (Stockhaus et al. (1987) Proc. Natl. Acad. Sci. USA 84: 7943-7947; Stockhaus et al. (1989) EMBO J. 8:2445-2451). Promoters can also be used which are active during the plant transformation, the plant regeneration or specific stages of these processes, such as cell division-specific promoters such as the Histon H3 promoter (Kapros et al. (1993) In Vitro Cell Cev. Biol. Plant 29:27-32) or the chemically induceable Tet-repressor system (Gatz et al. (1991) Mol. Gen. Genet. 227:229-237). Other suitable promoters can be taken from the literature, e.g. Ward (1993, Plant Mol. Biol. 22:361-366). The same applies for induceable and cell- or tissue-specific promoters, such as meristem-specific promoters, which have also been described in the literature and are suitable within the framework of the invention.

Other induceable promoters include pathogen-inducible promoters such as the ACMV virion sense promoter (Hong et al., 1996, Virology, 220:119-227) which is induced by the gene product AC2. Fungus induced promoters are also especially suitable for the present invention. Moreover, all promoters of such proteins which are induced in pathogen-infested tissues, such as phenylalanine ammonium lyase, chalcone synthase, hydroxyproline-rich glycoprotein, extensin, pathogenesis-related proteins (e.g. PR-1a) and wound-induceable protease inhibitors (U.S. Pat. No. 6,013,864), are suitable. Furthermore, leaf specific promoters, such as promoters from photosynthetic tissue (e.g. CAP promoter, RBCS promoter, GAPA promoter, GAPB promoter, ST-LS1 promoter etc.) are especially suitable for the present invention.

Moreover, the average person skilled in the art is able to isolate additional suitable promoters by means of routine methods. The person skilled in the art, with the help of established molecular biology methods, e.g. hybridization experiments or DNA-protein-binding studies, can thus identify leaf-specific regulatory nucleic acid elements. In so doing, e.g. in a first step the whole poly(A)⁺-RNA is isolated from the leaf tissue of the required organism from which the regulatory sequences are to be isolated, and a cDNA library is generated. In a second step, and with the help of cDNA clones which are based on poly(A)⁺-RNA molecules from a non-leaf tissue, those clones, the corresponding poly(A)⁺-RNA molecules of which only accumulate in the tissue of the leaf, are identified from the first library by means of hybridization. Finally, with the help of these cDNAs identified in this way, promoters are isolated which are equipped with leaf-specific regulatory elements. Other methods based on PCR are available to the person skilled in the art for the isolation of appropriate leaf-specific promoters.

Another embodiment uses the promoter of the class I patatin gene B33 from potato. Other favoured promoters are those which are particularly active in fruits. These include, for example, the promoter of a polygalacturonase gene, e.g. from tomato, which mediates expression during the maturation of tomato fruits (Nicho lass et al.) (1995) Plant Mol. Biol. 28:423-435; this state of the art describes the analysis of promoter/GUS fusion constructs), the promoter of an ACC oxidase, e.g. from apple, which mediates maturity and fruit specificity in transgenic tomatoes (Atkinson et al. (1998) Plant Mol. Biol. 38:449-460; this state of the art also discloses promoter/GUS expression analyses), or the 2A11 promoter from tomato (van Haaren et al. (1991) Plant Mol. Biol. 17:615-630, also describes promoter/GUS fusions).

Also in the case of fruit-specific promoters, the person skilled in the art can take other suitable promoters from the literature, or as described above for leaf-specific promoters, isolate them by means of routine methods.

The person skilled in the art knows that the use of inducible promoters allows the production of plants and plant cells which only transiently express, and so only transiently silence the sequences according to the invention. Such a transient expression allows the production of plants which only show transient pathogen resistance. Such a transient resistance can e.g. be desirable if there is the risk of pathogen contamination and the plants therefore need to be resistant to the pathogen only for a particular length of time. The person skilled in the art is aware of other situations in which transient resistance is desirable. The person skilled in the art is also aware that, by the use of vectors which do not stably replicate in plant cells and which carry the respective sequences for the silencing of MLO proteins, he can achieve transient expression and therefore also transient silencing and transient resistance.

For the introduction of DNA into a plant host cell, there are a number of well-known techniques available, whereby the person skilled in the art can determine the appropriate method in each case without any problem. These techniques include the transformation of plant cells with T-DNA by using Agrobacterium tumefaciens or Agrobacterium rhizogenes as a transformation agent, the fusion of protoplasts, the direct gene transfer of isolated DNA into protoplasts, the electroporation of DNA, the introduction of DNA by means of the biolistic method, as well as other possibilities. In so doing, both stable and transient transformants can be generated.

With the injection and electroporation of DNA into plant-cells there are no special requirements per se for the plasmids used. The same applies for direct gene transfer. Simple plasmids, such as pUC derivates can be used. If, however, whole plants are to be regenerated from such transformed cells, the presence of a selectable marker gene is necessary. The person skilled in the art is acquainted with the current selection markers, and he will have no problem in selecting an appropriate marker. Standard selection markers are those which mediate resistance to a biocide or an antibiotic such as kanamycin, G418, bleomycin, hygromycin, methotrexat, glyphosat, streptomycin, sulfonyl urea, gentamycin or phosphinotricin and suchlike, to the transformed plant cell.

Dependent upon the method of introduction of the desired gene into the plant cell, other DNA sequences may be required. For example, if the Ti or Ri plasmid is used for the transformation of the plant cell, at least the right flanking region, often however the right and the left flanking region of the T-DNA contained in the Ti or Ri plasmid must be linked as a flanking region with the gene to be introduced.

If agrobacteria are used for the transformations, the DNA to be introduced must be cloned in special plasmids, either in an intermediary or in a binary vector. Based on sequences which are homologous to sequences in the T-DNA, the intermediary vectors can be integrated into the Ti or Ri plasmid of the agrobacteria by homologous recombination. This plasmid also contains the vir region necessary for the transfer of the T-DNA. Intermediary vectors cannot replicate in agrobacteria. By means of a helper plasmid, the intermediary vector can be transferred to Agrobacterium tumefaciens (conjugation). Binary vectors can replicate in E. coli as well as in agrobacteria. They contain a selection marker gene and a linker or polylinker which are framed by the right and left T-DNA border regions. They can be transformed directly into the agrobacteria (Holsters et al. (1978), Molecular and General Genetics 163, 181-187). The agrobacterium serving as a host cell should contain a plasmid which carries a vir region. The vir region is necessary for the transfer of the T-DNA into the plant cell. T-DNA can also be present. This type of transformed agrobacterium is used for the transformation of plant cells.

The use of T-DNA for the transformation of plant cells has been intensively investigated and is described sufficiently in EP 120 515.

For the transfer of DNA into the plant cell, plant explants can be cultivated specifically for this purpose with Agrobacterium tumefaciens or Agrobacterium rhizogenes. From the infected plant material (for example, pieces of leaf, stem segments, roots, but also protoplasts or suspension-cultivated plant cells) whole plants can be regenerated in an appropriate medium which can contain antibiotics or biocides for the selection of transformed cells. The regeneration of the plants takes place according to standard regeneration methods and using the common nutrient solutions. The plants and plant cells obtained in this way can by examined for the presence of the DNA introduced.

The person skilled in the art is acquainted with other possibilities for the introduction of foreign DNA using the biolistic method or by protoplast transformation (see L. Willmitzer (1993) Transgenic Plants in: Biotechnology, A Multi-Volume Comprehensive Treatise (publisher: H. J. Rehm et al.), volume 2, 627-659, VCH Weinheim, Germany).

Whereas the transformation of dicotyledenous plants or their cells by means of Ti plasmid vector systems with the help of Agrobacterium tumefaciens is well established, new work points to the fact that monocotyledonous plants or their cells are also very accessible to transformation by means of vectors based on agrobacteria (see e.g. Chan et al. (1993), Plant Mol. Biol. 22, 491-506).

Alternative systems for the transformation of monocotyledonous plants or their cells are transformation by means of the biolistic approach (Wan and Lemaux (1994) Plant Physiol. 104, 37-48; Vasil et al. (1993) Bio/Technology 11, 1553-1558; Ritala et al. (1994) Plant Mol. Bio. 24, 317-325; Spencer et al. (1990), Theor. Appl. Genet. 79, 625-631), protoplast transformation, electroporation of partially permeabilised cells as well as the introduction of DNA by means of glass tissues.

The transformed cells grow within the plant in the normal way (see also McCormick et al. (1986), Plant Cell Reports 5, 81-84). The resulting plants can be raised in the normal way and be crossed with plants which have the same transformed genetic disposition or other genetic dispositions. The resulting hybrid individuals have the respective phenotypical properties.

Two or more generations should be raised in order to ensure that the phenotypical feature remains stable and is inherited. Seeds should be harvested as well so as to ensure that the respective phenotype or other characteristics are maintained.

Similarly, by using the standard methods, transgenic lines can be determined which are homozygous for the new nucleic acid molecules and their phenotypical characteristics with regard to a present or non-present pathogen responsiveness is investigated and compared with that from hemizygous lines.

Of course, plant cells which contain the nucleic acid molecules according to the invention and plant cells (including protoplasts, calli, suspension cultures and suchlike) can further be cultivated.

The vectors described above can be transferred to plant cells in various ways. Whether the vectors are in linear or circular form depends upon the application in question. The person skilled in the art knows whether and when he can use respective linearized vectors or not. For example, the person skilled in the art knows that, for the production of specific knockouts of genes for MLO proteins by homologous recombination, it can suffice to linearize the corresponding vectors and inject them into the plants or plant cells.

Furthermore, the invention is also directed to a transgenic plant or plant cell having an increased resistance against soybean rust, characterized in that the content and/or the activity of at least one MLO protein is altered in comparison to wild type plants or plant cells, respectively, This plant can, e.g., be produced by any one of the methods which have been described above. “Transgenic plants” and the transgenic plant cells can signify any monocotyledonous or dicotyledonous plant or plant cell, preferably agricultural plants or cells from agricultural plants. The invention is further directed to transgenic parts of this plant such as leaves and blossoms, transgenic propagation material such as protoplasts, calli, fruit, seeds, tubers, rootstocks, germs, pollen, cuttings, and transgenic progeny of the plant.

According to one preferred embodiment, the content and/or the activity of at least one MLO protein is decreased in comparison to wild type plants or plant cells.

According to another preferred embodiment, the content and/or the activity of at least one MLO protein is increased in comparison to wild type plants or plant cells.

Further subject-matter of the present invention involves plant cells and plants in which the endogenous genes of MLO proteins have mutations, i.e. substitutions, insertions and/or deletions, which lead to the fact that the endogenous MLO proteins expressed are no longer able, or only able under certain circumstances, to interact with their endogenous cellular or pathologic binding partners. Plants or plant cells, which contain these endogenous gene copies for MLO proteins showing mutations, can be distinguished by increased transient or permanent resistance against soybean rust. Such plants and plant cells which, unlike the plants and plant cells specified above, are not transgenic can be produced by conventional mutagenesis.

Modulation of the expression of the endogenous plant MLO proteins can e.g. mean that by means of mutations in regulatory DNA elements of the genes of the MLO proteins, such as promoters, enhancers or generally so-called “upstream activating sequences”, the expression of endogenous MLO proteins is down-regulated.

Within the framework of the present invention, the modulation of the binding characteristics of the MLO proteins means that the above-specified types of mutation lead to a change of the binding characteristics of the endogenous MLO proteins their endogenous cellular or pathologic binding partners. A combination of the modulation of the expression and the binding characteristics of the MLO proteins is also possible.

For example, plants or plant cells can have mutations in the gene sequences for MLO proteins which lead to the reduction of the expression of these proteins. Other plants or plant cells have mutations which lead to the dominant negative mutants described above. In both cases, plants with increased resistance against soybean rust are obtained.

The person skilled in the art is aware that e.g. plants and plant cells can also be produced by mutagenesis which, because of mutations in enhancer and/or promoter sequences of the genes for plant MLO proteins, show a reduction of the expression of these proteins, and at the same time show mutations in the coding regions of the genes encoding MLO proteins, which give rise to the fact that the remaining expressed MLO proteins can no longer, or only to a limited extent, interact with the fungal and/or other cellular binding partners. On the other hand, respective mutations in enhancer and/or promoter sequences and in the coding sequences can have the effect that a dominant negative mutant of plant MLO proteins, as described above, which is no longer, or only to a very limited extent, able to interact with fungal and/or normal cellular interaction partners, is overexpressed, and so the competition reaction described above comes about.

Preferably, the non-transgenic plants and plant cells according to the invention, which are distinguished by a modulation of the expression and/or the binding characteristics of the endogenous MLO proteins and have permanent or transient resistance against soybean rust, are produced by means of the so-called “TILLING” method (Targeting Induced Local Lesion in Genomes). This method has been described in detail in Colbert et al. (2001, Plant Physiology, 126, 480-484), McCallum et al. (2000, Nat. Biotechnol., 18, 455-457) and McCallum et al. (2000, Plant Physiology, 123, 439-442). The above-specified references are introduced here as explicit disclosure with regard to the “TILLING” method.

The TILLING method is a strategy of the so-called reverse genetics which combines the production of high frequencies of point mutations in mutagenized plant collections, e.g. by means of chemical mutagenesis with ethyl methane sulphonate (EMS), with the fast systematic identification of mutations in target sequences. First of all, the target sequence is amplified via PCR in DNA pools of mutagenized M2 populations. Denaturation and annealing reactions of the heteroallelic PCR products allow the formation of heteroduplexes, wherein one DNA strand originates from the mutated and the other from the “wild-type” PCR product. A so-called mismatch then takes place at the site of the point mutation, which can be identified either by means of denaturating HPLC (DHPLC, McCallum et al., 2000, Plant Physiol., 123, 439-442) or with the Cell mismatch detection system (Oleykowsky et al., 1998, Nucl. Acids Res. 26, 4597-4602). Cell is an endonuclease which recognizes the mismatches in heteroduplex DNA and specifically cleaves the DNA at these sites. The cleavage products can then be separated and detected by means of automated sequencing gel electrophoresis (Colbert et al., 2001, vide supra). Following identification of target gene-specific mutations in a pool, individual DNA samples are analyzed accordingly in order to isolate the plant with the mutation. In this way, the identification of the mutagenized plant cells or plants can be made with the plants and plants cells according to the invention after the production of the mutagenized plant populations by the use of primer sequences targeted at MLO proteins. The TILLING method is generally applicable for all plants and so the cultivated and agricultural plants specified above are suitable for the method according to the invention.

Therefore the present invention is also directed to a soybean rust resistant plant or plant cell, characterized in that it has been produced by the TILLING method and that it contains mutations in the coding and/or regulatory sequences of at least one gene encoding an MLO protein which cause an alteration in the content and/or the activity of the at least one MLO protein in comparison to wild type plants or plant cells.

The present invention is further directed to the use of at least one nucleic acid molecule, comprising:
a) at least one sequence which is identical, homologous or complementary to a sequence encoding an endogenous Mlo or fragments thereof,
b) at least one sequence encoding a non-functional Mlo or a fragment thereof which has at least one point mutation, deletion and/or insertion,
c) at least one sequence encoding a recombinant antibody which is specific for an endogenous Mlo and which prevents the cellular function of the Mlo,
d) at least one sequence encoding an Mlo inhibitor which prevents the cellular function an Mlo, and/or
e) at least one sequence encoding an Mlo and/or a functionally equivalent fragment thereof and/or a functionally equivalent derivative thereof
   for increasing the resistance against soybean rust in transgenic plants and/or plant cells.

The invention is also directed to an expression vector, comprising the following nucleic acid sequences in 5′-3′ orientation:
a) a promoter sequence which is functionally active in plants,
b) operatively linked thereto a sequence
   being identical, homologous or complementary to a sequence encoding an endogenous Mlo or fragments thereof,
   encoding a non-functional Mlo or a fragment thereof which has at least one point mutation, deletion and/or insertion,
   encoding a recombinant antibody which is specific for an endogenous Mlo and which prevents the cellular function of the Mlo,
   encoding an Mlo inhibitor which prevents the cellular function an Mlo, and/or
   encoding an Mlo and/or a functionally equivalent fragment thereof and/or a functionally equivalent derivative thereof,
c) optionally, operatively linked thereto a termination sequence which is functionally active in plants.

Finally, the invention is also directed to an isolated nucleic acid molecule comprising at least one nucleic acid sequence, selected from the group consisting of:
a) a nucleotide sequence according to SEQ ID NOs: 3, 5, 6, 8 or 10 or fragments thereof,
b) a nucleotide sequence which encodes a polypeptide having an amino acid sequence according to any one of SEQ ID NOs: 4, 7, 9 or 11 or fragments thereof,
c) a nucleotide sequence which is essentially homologous to any one of the nucleotide sequences of a) or b),
d) a nucleotide sequence which can hybridize under stringent conditions with any one of the nucleotide sequences of a), b) or c),
   wherein the nucleic acid sequence encodes an MLO protein.

The skilled person is aware of the fact that the term “nucleotide sequence” of item a) preferably refers to the coding parts of SEQ ID NOs: 3, 5, 6, 8 or 10, i.e. the parts which encode an MLO protein, and not the regulatory parts which are usually located upstream and downstream of the coding region. However, the 5′ and 3′ untranslated regions of SEQ ID NOs: 3, 5, 6, 8 or 10 can also be included in the nucleic acid molecule.

An “isolated nucleic acid molecule” means a molecule which is separated from the other nucleic acid molecule which are present in the natural source of the nucleic acid. Preferably, an isolated nucleic acid molecule comprises no sequences which are naturally flanking the genomic DNA of the organism from which the molecule originates. In some embodiments, the isolated nucleic acid molecule can include, e.g., less than about 5 kb, 4 kb, 3 kb, 2 kb, 1 kb, 0.5 kb or 0.1 kb of nucleic acid sequences which are naturally flanking the genomic DNA of the cell from which the molecule originates.

An “MLO protein” has the biologic activity that the alteration of its content and/or activity within a plant or plant cell leads to in increased resistance of the plant or plant cell against soybean rust. The biological activity (or “cellular function”) is especially meant to be the interactions of the MLO protein with its pathogenic or physiologic binding partners, i.e. preferably its interactions with calmodulin and/or ROR2. Therefore, a “fragment” of a nucleotide sequence which is part of the above-mentioned isolated nucleic acid molecule is limited to those fragments which encode an MLO protein having this biologic activity. Usually, the fragments lack nucleotides at the 5′ end or at the 3′ end. Preferably the fragment has at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% of the length of the “whole” MLO encoding sequence.

A nucleotide sequence which is “essentially homologous” to another nucleotide sequence means, in the scope of the present invention, that the sequence is at least 40% or 50%, preferably at least 55% or 60%, more preferably at least 65% or 70%, especially preferably at least 75% or 80%, particularly preferably at least 85% or 90%, and most preferably at least 92%, 94%, 95%, 96%, 97%, 98% or 99% similar to the other sequence or fragments. Preferably, this homology is determined over the whole sequence length of the nucleotide sequence.

Of course, the nucleotide sequences which are “essentially homologous” or which can “hybridize under stringent conditions” (see items c) and d) above) also need to encode a functional MLO protein according to the invention, i.e. an MLO protein having a biologic activity as described above.

### EXAMPLES

Barley mlo mutation mlo5 was used in the present invention. Mlo5 is a complete null mutant which was obtained by the exchange of Met-1 (ATG) to Ile. The sequence of the wild-type MLO protein is depicted in SEQ ID NO: 2.

Barley cultivar “Ingrid” wild type and mlo5 mutant were used. The seeds were provided by the division APR/HS of the BASF AG (Agrarzentrum, Limburgerhof). The breeding was performed for 7 days in climatic exposure test cabinets at controlled conditions, i.e. a temperature of 22° C. and a day/night rhythm of 12 hours. The plants were inoculated with P. pachyrhizi 7 days after the sowings.

The soybean rust fungus was a wild isolate from Brazil.

In order to obtain appropriate spore material for the inoculation, soybean leaves which had been infected with soybean rust 15-20 days ago, were taken 2-3 days before the inoculation and transferred to agar plates (1% agar in H₂O). The leaves were placed with their upper side onto the agar, which allows the fungus to grow through the tissue and to produce very young spores. For the inoculation solution, the spores were knocked off the leaves and were added to a Tween-H₂O solution. The counting of spores was performed under a light microscope by means of a Thoma counting chamber. For the inoculation of the plants, the spore suspension was added into a compressed-air operated spray flask and applied uniformly onto the plants or the leaves until the leaf surface was well moisturized. For the microscopy, a density of 10×10⁵ spores/ml was used. The inoculated plants were placed for 24 hours in a greenhouse chamber with an average of 22° C. and >90% of air humidity. The inoculated leaves were incubated under the same conditions in a closed Petri dish on 0.5% plant agar. The following cultivation was performed in a chamber with an average of 25° C. and 70% of air humidity.

For the evaluation of the pathogen development, the inoculated leaves of barley “Ingrid” wild-type and barley “Ingrid” mlo5 were stained with aniline blue. The same protocol can also be used for soybean.

The aniline blue staining serves for the detection of fluorescent substances. During the defense reactions in host interactions and non-host interactions, substances such as phenols, callose or lignin accumulate or are produced and are incorporated at the cell wall either locally in papillae or in the whole cell (hypersensitive reaction, HR). Complexes are formed in association with aniline blue, which lead e.g. in the case of callose to yellow fluorescence. The leaf material was transferred to falcon tubes or dishes containing destaining solution II (ethanol/acetic acid 6/1) and was incubated in a water bath at 90° C. for 10-15 minutes. The destaining solution II was removed immediately thereafter, and the leaves were washed 2× with water. For the staining, the leaves were incubated for 1.5-2 hours in staining solution II (0.05% aniline blue=methyl blue, 0.067 M di-potassium hydrogen phosphate) and analyzed by microscopy immediately thereafter.

The different interaction types were evaluated (counted) by microscopy. An Olympus UV microscope BX61 (incident light) and a UV Longpath filter (excitation: 375/15, Beam splitter: 405 LP) were used. After aniline blue staining, the spores appear blue under UV light. The papillae can be recognized beneath the fungal appressorium by a green/yellow staining The hypersensitive reaction (HR) is characterized by a whole cell fluorescence.

The results are shown in Table 3 as well as in FIG. 1.

Two main stages of plant resistance against fungal growth can be discriminated. 1. The formation of a papilla as a mechanical barrier on the inner side of the cell wall under the appressorium prevents the infection of the cell and the further growth of the fungus. 2. The death of the infected cell, called hypersensitive reaction (HR), is another means of resistance against the fungus.

FIG. 1 shows a significant increase of the rate of papillae formation in the barley “Ingrid” mlo5 compared to the wild-type.

TABLE 3
Barley-lines infected with soybean rust fungus

| | | | | | | | papillae | |
|---|---|---|---|---|---|---|---|---|
| | barley | | spores with | appressorium | HR | appressorium | formation | HR |
| exp.# | Ingrid | spores | appressorium | with papillae | reaction | formation [%] | [%] | [%] |
| 1 | wt | 570 | 479 | 181 | 142 | 84.0 | 37.8 | 29.6 |
| | mlo5 | 606 | 536 | 274 | 92 | 88.4 | 51.1 | 17.2 |
| 2 | wt | 763 | 688 | 352 | 123 | 90.2 | 51.2 | 17.9 |
| | mlo5 | 795 | 657 | 445 | 127 | 82.6 | 67.7 | 19.3 |
| 3 | wt | 638 | 523 | 212 | 74 | 82.0 | 40.5 | 14.1 |
| | mlo5 | 534 | 470 | 328 | 37 | 88.0 | 69.8 | 7.9 |
| 4 | wt | 933 | 870 | 358 | 60 | 93.2 | 41.1 | 6.9 |
| | mlo5 | 894 | 793 | 510 | 26 | 88.7 | 64.3 | 3.3 |
| 5 | wt | 692 | 643 | 258 | 44 | 92.9 | 40.1 | 6.8 |
| | mlo5 | 745 | 667 | 476 | 60 | 89.5 | 71.4 | 9.0 |
| 6 | wt | 491 | 355 | 112 | 162 | 72.3 | 31.5 | 45.6 |
| | mlo5 | 707 | 500 | 230 | 148 | 70.7 | 46.0 | 29.6 |
| total | wt | 4087 | 3558 | 1473 | 605 | 87.1 | 41.4 | 17.0 |
| | mlo5 | 4281 | 3623 | 2263 | 490 | 84.6 | 62.5 | 13.5 |

The standard error for the papillae formation percentage is 0.02592647 in wt barley and 0.04322237 in mlo5 barley. The value for the T test is 0.001735842.

### DESCRIPTION OF THE FIGURES

FIG. 1 Rate of papillae formation (%) of barley “Ingrid” wild type and barley “Ingrid” mlo5 mutant after infection with the soybean rust fungus.

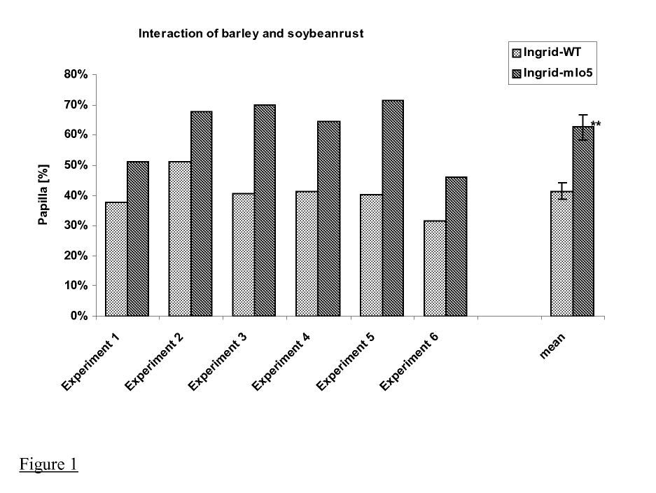

FIG. 2a GmMlo1—Soy (full-length) nucleic acid sequence (SEQ ID NO: 3) GmMlo1—Soy (full-length) amino acid sequence (SEQ ID NO: 4)

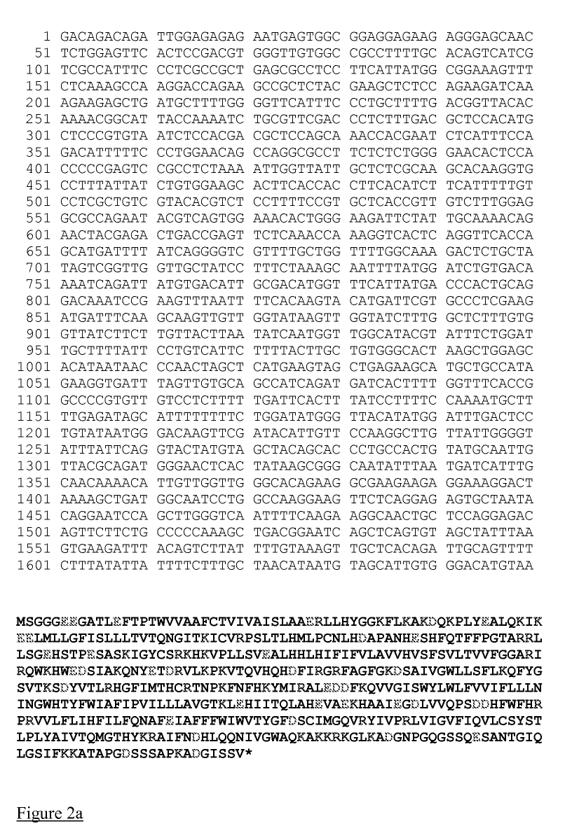

FIG. 2b GmMlo2 (genomic)—Soy partial nucleic acid sequence (SEQ ID NO: 5) GmMlo2 (EST)—Soy partial nucleic acid sequence (SEQ ID NO: 6) GmMlo2 (EST)—Soy partial amino acid sequence (SEQ ID NO: 7)

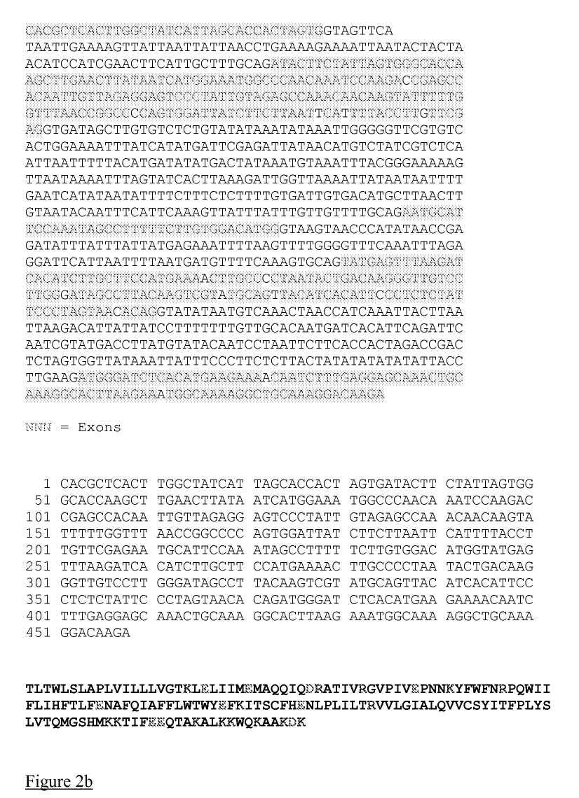

FIG. 2c GmMlo3.1—Soy (full length) nucleic acid sequence (SEQ ID NO: 8) GmMlo3.1—Soy (full length) amino acid sequence (SEQ ID NO: 9)

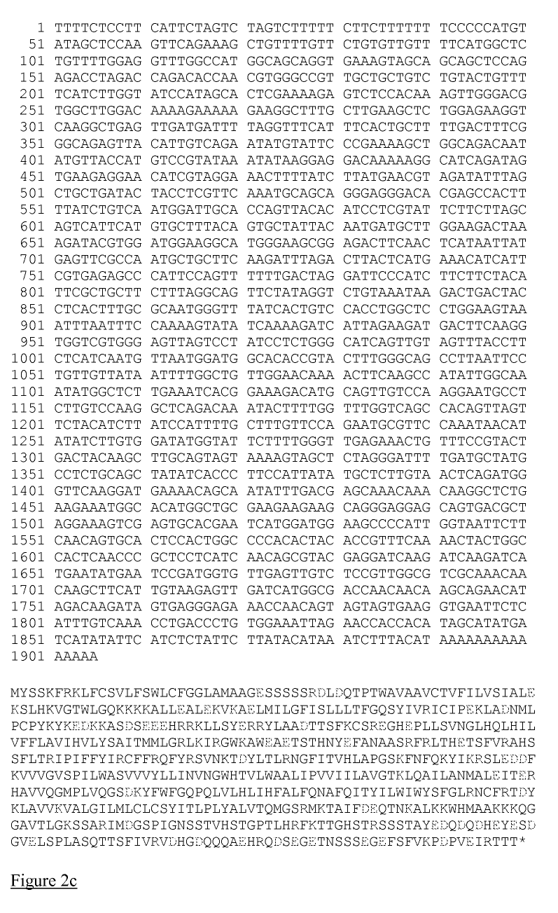

FIG. 2d GmMlo3.2 (EST)—Soy nucleic acid sequence (SEQ ID NO: 10) GmMlo3.2—Soy theoretical amino acid sequence (SEQ ID NO: 11)

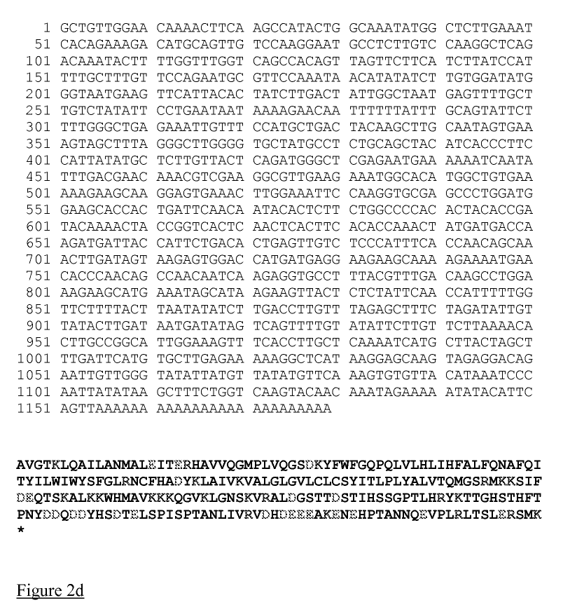

1-53. (canceled)

54. A method of increasing resistance against soybean rust in transgenic plants and/or plant cells, comprising altering the content and/or the activity of at least one MLO protein in a transgenic and/or plant cell in comparison to a wild type plant or plant cell, respectively.

55. The method of claim 54, wherein the content and/or the activity of at least one endogenous MLO is decreased in comparison to the wild type.

56. The method of claim 55, wherein the content and/or the activity of at least one endogenous MLO is decreased by transferring at least one nucleic acid molecule comprising at least one sequence which is identical, homologous or complementary to a sequence encoding the endogenous MLO or fragment thereof to the plant cell.

57. The method of claim 56, wherein a part of the transferred nucleic acid molecule is at least 50% homologous to a sequence encoding the endogenous MLO or fragment thereof.

58. The method of claim 56, wherein the decrease of the content and/or the activity of the at least one endogenous MLO is achieved by RNA interference (RNAi), an antisense construct, a co-suppression construct, post-transcriptional gene silencing (PTGS), a ribonuclease P construct, homologous recombination, a ribozyme construct or virus induced gene silencing (VIGS).

59. The method of claim 55, wherein the content and/or the activity of at least one endogenous MLO is decreased by the expression of at least one non-functional MLO or a fragment thereof which has at least one point mutation, deletion and/or insertion.

60. The method of claim 59, wherein the at least one point mutation, deletion and/or insertion of the non-functional MLO prevent the cellular function of MLO.

61. The method of claim 55, wherein the content and/or the activity of at least one endogenous MLO is decreased by the expression of at least one recombinant antibody which is specific for at least one endogenous MLO and which prevents the cellular function of the MLO.

62. The method of claim 55, wherein the content and/or the activity of at least one endogenous MLO is decreased by the expression of at least one MLO inhibitor which prevents the cellular function of at least one MLO.

63. The method of claim 54, wherein the MLO is a plant MLO.

64. The method of claim 54, wherein the MLO comprises an amino acid sequence selected from the group consisting of the amino acid sequence as depicted in SEQ ID NO: 2, 4, 7, 9, 11, 14, 16, 18, 20, 22, 24, 26, 28, 30, 32, 34, 36, 38, 40, 42, 44, 46, and 48, or an MLO having an amino acid sequence which is essentially a functionally equivalent thereof.

65. The method of claim 54, wherein the plant is a dicotyledonous plant.

66. The method of claim 54, wherein the plant is a monocotyledonous plant.

67. A transgenic plant or plant cell having an increased resistance against soybean rust or transgenic part or transgenic propagation material thereof, wherein the content and/or the activity of at least one MLO protein is altered in comparison to a wild type plant or plant cell, respectively.

68. The transgenic plant or plant cell of claim 67, wherein the content and/or the activity of at least one endogenous MLO protein is decreased in comparison to a wild type plant or plant cell, respectively.

69. The transgenic plant or plant cell of 68, wherein the content and/or the activity of at least one MLO protein is increased in comparison to a wild type plant or plant cell, respectively.

70. A transgenic plant or plant cell having an increased resistance against soybean rust produced by the method of claim 54 or a transgenic part or transgenic propagation material thereof.

71. The method of claim 59, wherein the at least one point mutation, deletion and/or insertion of the nun-functional MLO inhibit the interaction of MLO with its pathogenic or physiologic binding partner.

72. The method of claim 71, wherein the pathogenic or physiologic binding partner is Ror2 and/or calmodulin.

73. The method of claim 55, wherein the content and/or the activity of at least one endogenous MLO is decreased by the expression of at least one recombinant antibody which is specific for at least one endogenous MLO and which inhibits the interaction of the MLO with its pathogenic or physiologic binding partner.

74. The method of claim 73, wherein the pathogenic or physiologic binding partner is Ror2 and/or calmodulin.

75. The method of claim 55, wherein the content and/or the activity of at least one endogenous MLO is decreased by the expression of at least one MLO inhibitor which inhibits the interaction of the MLO with its pathogenic or physiologic binding partner.

76. The method of claim 75, wherein the pathogenic or physiologic binding partner is Ror2 and/or calmodulin.

77. The method of claim 63, wherein the plant MLO is selected from the group consisting of Hordeum vulgare (barley) MLO, Oryza sativa (rice) MLO, Arabidopsis thaliana MLO, Linum usitatissimum (flax) MLO, Triticum aestivum (wheat) MLO, and Glycine max (soy) MLO, or an MLO which is essentially functionally equivalent to any one of said MLO proteins.

78. The method of claim 65, wherein the dicotyledonous plant is soybean, alfalfa, cotton, rapeseed, tomato, sugar beet, potato, sunflower, pea, an ornamental plant, tobacco, clover (Trifolium sp.), Kudzu (Pueraria lobata), a tree, or a legume.

79. The method of claim 66, wherein the monocotyledonous plant is Hordeum (barley), Avena (oat), Triticum (wheat), Secale (rye), Oryza (rice), Sorghum (millet), Zea (corn), Panicum, Pennisetum, or Setaria.

80. The transgenic plant or plant cell of claim 67, wherein the transgenic part or transgenic propagation material thereof is a leaf, blossom, protoplast, callus, fruit, seed, tuber, rootstock, germ, pollen, cutting, or transgenic progeny of the plant.

81. The transgenic plant or plant cell of claim 70, wherein the transgenic part or transgenic propagation material thereof a leaf, blossom, protoplast, callus, fruit, seed, tuber, rootstock, germ, pollen, cutting, or transgenic progeny of the plant.


SEQUENCE LISTING (48 sequences)

SEQ ID NO: 1 - DNA, 1602 bp, Hordeum vulgare
atgtcggacaaaaaaggggtgccggcgcgggagctgccggagacgccgtcgtgggcggtggcggtggtcttcgccgccatggtgctcgtgtccgtcctcatggaacacggcctccacaagctcggccattggttccagcaccggcacaagaaggccctgtgggaggcgctggagaagatgaaggcggagctcatgctggtgggcttcatatccctgctcctcatcgtcacgcaggaccccatcatcgccaagatatgcatctccgaggatgccgccgacgtcatgtggccctgcaagcgcggcaccgagggccgcaagcccagcaagtacgttgactactgcccggagggcaaggtggcgctcatgtccacgggcagcttgcaccagctgcacgtcttcatcttcgtgctcgcggtcttccatgtcacctacagcgtcatcaccatagctctaagccgtctcaaaatgagaacatggaagaaatgggagacagagaccacctccttggaataccagttcgcaaatgatcctgcacggttccggttcacgcaccagacgtcgttcgtgaagcgccacctgggcctctccagcacccctggcatcagatgggtggtggccttcttcaggcagttcttcaggtcagtcaccaaggtggactacctgaccttgagggcaggcttcatcaacgcgcatttgtcgcaaaacagcaagttcgacttccacaagtacatcaagaggtcgatggaggacgacttcaaggtcgtcgtcggcatcagcctcccgctgtggggtgtggcgatcctcaccctcttccttgacatcaatggggttggcacgctcatctggatttctttcatccctctcgtgatcctcttgtgtgttggaaccaagctggagatgatcatcatggagatggccctggagatccaggaccgggcgagcgtcatcaagggggcccccgtggtcgagcccagcaacaagttcttctggttccaccgccccgactgggtcctcttcttcatacacctgacgttgttccagaacgcgtttcagatggcgcattttgtgtggacagtggccacgcccggcttgaagaaatgctaccacacgcagatcgggctgagcatcatgaaggtggtggtggggctagctctccagttcctctgcagctatatgaccttccccctctacgcgctcgtcacacagatgggatcaaacatgaagaggtccatcttcgacgagcagacgtccaaggcgctcaccaactggcggaacacggccaaggagaagaagaaagtccgagacacggacatgctgatggctcagatgatcggcgacgcaacaccgagccgaggctcgtcgccgatgccgagccggggctcatcacccgtgcacctgcttcacaagggcatggggcggtcggacgacccccagagcgcgcccacctcgccaaggacccagcaggaggctagggacatgtacccggttgtggtggcgcacccggtgcacagactaaatcctaacgacaggaggaggtccgcctcgtcgtcggccctcgaagccgacatccccagtgcagatttttccttcagccagggatga

SEQ ID NO: 2 - protein, 533 aa, Hordeum vulgare
MSDKKGVPARELPETPSWAVAVVFAAMVLVSVLMEHGLHKLGHWFQHRHKKALWEALEKMKAELMLVGFISLLLIVTQDPIIAKICISEDAADVMWPCKRGTEGRKPSKYVDYCPEGKVALMSTGSLHQLHVFIFVLAVFHVTYSVITIALSRLKMRTWKKWETETTSLEYQFANDPARFRFTHQTSFVKRHLGLSSTPGIRWVVAFFRQFFRSVTKVDYLTLRAGFINAHLSQNSKFDFHKYIKRSMEDDFKVVVGISLPLWGVAILTLFLDINGVGTLIWISFIPLVILLCVGTKLEMIIMEMALEIQDRASVIKGAPVVEPSNKFFWFHRPDWVLFFIHLTLFQNAFQMAHFVWTVATPGLKKCYHTQIGLSIMKVVVGLALQFLCSYMTFPLYALVTQMGSNMKRSIFDEQTSKALTNWRNTAKEKKKVRDTDMLMAQMIGDATPSRGSSPMPSRGSSPVHLLHKGMGRSDDPQSAPTSPRTQQEARDMYPVVVAHPVHRLNPNDRRRSASSSALEADIPSADFSFSQG

SEQ ID NO: 3 - DNA, 1650 bp, Glycine max
gacagacagattggagagagaatgagtggcggaggagaagagggagcaactctggagttcactccgacgtgggttgtggccgccttttgcacagtcatcgtcgccatttccctcgccgctgagcgcctccttcattatggcggaaagtttctcaaagccaaggaccagaagccgctctacgaagctctccagaagatcaaagaagagctgatgcttttggggttcatttccctgcttttgacggttacacaaaacggcattaccaaaatctgcgttcgaccctctttgacgctccacatgctcccgtgtaatctccacgacgctccagcaaaccacgaatctcatttccagacatttttccctggaacagccaggcgccttctctctggggaacactccacccccgagtccgcctctaaaattggttattgctctcgcaagcacaaggtgcctttattatctgtggaagcacttcaccaccttcacatcttcatttttgtcctcgctgtcgtacacgtctccttttccgtgctcaccgttgtctttggaggcgccagaatacgtcagtggaaacactgggaagattctattgcaaaacagaactacgagactgaccgagttctcaaaccaaaggtcactcaggttcaccagcatgattttatcaggggtcgttttgctggttttggcaaagactctgctatagtcggttggttgctatcctttctaaagcaattttatggatctgtgacaaaatcagattatgtgacattgcgacatggtttcattatgacccactgcaggacaaatccgaagtttaattttcacaagtacatgattcgtgccctcgaagatgatttcaagcaagttgttggtataagttggtatctttggctctttgtggttatcttcttgttacttaatatcaatggttggcatacgtatttctggattgcttttattcctgtcattcttttacttgctgtgggcactaagctggagcacataataacccaactagctcatgaagtagctgagaagcatgctgccatagaaggtgatttagttgtgcagccatcagatgatcacttttggtttcaccggccccgtgttgtcctctttttgattcactttatccttttccaaaatgcttttgagatagcattttttttctggatatgggttacatatggatttgactcctgtataatgggacaagttcgatacattgttccaaggcttgttattggggtatttattcaggtactatgtagctacagcaccctgccactgtatgcaattgttacgcagatgggaactcactataagcgggcaatatttaatgatcatttgcaacaaaacattgttggttgggcacagaaggcgaagaagaggaaaggactaaaagctgatggcaatcctggccaaggaagttctcaggagagtgctaatacaggaatccagcttgggtcaattttcaagaaggcaactgctccaggagacagttcttctgcccccaaagctgacggaatcagctcagtgtagctatttaagtgaagatttacagtcttattttgtaaagttgctcacagattgcagttttctttatattattttctttgctaacataatgtagcattgtgggacatgtaa

SEQ ID NO: 4 - protein, 506 aa, Glycine max
MSGGGEEGATLEFTPTWVVAAFCTVIVAISLAAERLLHYGGKFLKAKDQKPLYEALQKIKEELMLLGFISLLLTVTQNGITKICVRPSLTLHMLPCNLHDAPANHESHFQTFFPGTARRLLSGEHSTPESASKIGYCSRKHKVPLLSVEALHHLHIFIFVLAVVHVSFSVLTVVFGGARIRQWKHWEDSIAKQNYETDRVLKPKVTQVHQHDFIRGRFAGFGKDSAIVGWLLSFLKQFYGSVTKSDYVTLRHGFIMTHCRTNPKFNFHKYMIRALEDDFKQVVGISWYLWLFVVIFLLLNINGWHTYFWIAFIPVILLLAVGTKLEHIITQLAHEVAEKHAAIEGDLVVQPSDDHFWFHRPRVVLFLIHFILFQNAFEIAFFFWIWVTYGFDSCIMGQVRYIVPRLVIGVFIQVLCSYSTLPLYAIVTQMGTHYKRAIFNDHLQQNIVGWAQKAKKRKGLKADGNPGQGSSQESANTGIQLGSIFKKATAPGDSSSAPKADGISSV

SEQ ID NO: 5 - DNA, 1133 bp, Glycine max
cacgctcacttggctatcattagcaccactagtggtagttcataattgaaaagttattaattattaacctgaaaagaaaattaatactactaacatccatcgaacttcattgctttgcagatacttctattagtgggcaccaagcttgaacttataatcatggaaatggcccaacaaatccaagaccgagccacaattgttagaggagtccctattgtagagccaaacaacaagtatttttggtttaaccggccccagtggattatcttcttaattcattttaccttgttcgaggtgatagcttgtgtctctgtatataaatataaattgggggttcgtgtcactggaaaatttatcatatgattcgagattataacatgtctatcgtctcaattaatttttacatgatatatgactataaatgtaaatttacgggaaaaagttaataaaatttagtatcacttaaagattggttaaaattataataattttgaatcatataatattttctttctcttttgtgattgtgacatgcttaacttgtaatacaatttcattcaaagttatttatttgttgttttgcagaatgcattccaaatagcctttttcttgtggacatgggtaagtaacccatataaccgagatatttatttattatgagaaattttaagttttggggtttcaaatttagaggattcattaattttaatgatgttttcaaagtgcagtatgagtttaagatcacatcttgcttccatgaaaacttgcccctaatactgacaagggttgtccttgggatagccttacaagtcgtatgcagttacatcacattccctctctattccctagtaacacaggtatataatgtcaaactaaccatcaaattacttaattaagacattattatcctttttttgttgcacaatgatcacattcagattcaatcgtatgaccttatgtatacaatcctaattcttcaccactagaccgactctagtggttataaattatttcccttctcttactatatatatatattaccttgaagatgggatctcacatgaagaaaacaatctttgaggagcaaactgcaaaggcacttaagaaatggcaaaaggctgcaaaggacaaga

SEQ ID NO: 6 - DNA, 458 bp, Glycine max
cacgctcacttggctatcattagcaccactagtgatacttctattagtgggcaccaagcttgaacttataatcatggaaatggcccaacaaatccaagaccgagccacaattgttagaggagtccctattgtagagccaaacaacaagtatttttggtttaaccggccccagtggattatcttcttaattcattttaccttgttcgagaatgcattccaaatagcctttttcttgtggacatggtatgagtttaagatcacatcttgcttccatgaaaacttgcccctaatactgacaagggttgtccttgggatagccttacaagtcgtatgcagttacatcacattccctctctattccctagtaacacagatgggatctcacatgaagaaaacaatctttgaggagcaaactgcaaaggcacttaagaaatggcaaaaggctgcaaaggacaaga

SEQ ID NO: 7 - protein, 152 aa, Glycine max
TLTWLSLAPLVILLLVGTKLELIIMEMAQQIQDRATIVRGVPIVEPNNKYFWFNRPQWIIFLIHFTLFENAFQIAFFLWTWYEFKITSCFHENLPLILTRVVLGIALQVVCSYITFPLYSLVTQMGSHMKKTIFEEQTAKALKKWQKAAKDK

SEQ ID NO: 8 - DNA, 1905 bp, Glycine max
ttttctccttcattctagtctagtctttttcttctttttttcccccatgtatagctccaagttcagaaagctgttttgttctgtgttgttttcatggctctgttttggaggtttggccatggcagcaggtgaaagtagcagcagctccagagacctagaccagacaccaacgtgggccgttgctgctgtctgtactgttttcatcttggtatccatagcactcgaaaagagtctccacaaagttgggacgtggcttggacaaaagaaaaagaaggctttgcttgaagctctggagaaggtcaaggctgagttgatgattttaggtttcatttcactgcttttgactttcgggcagagttacattgtcagaatatgtattcccgaaaagctggcagacaatatgttaccatgtccgtataaatataaggaggacaaaaaggcatcagatagtgaagaggaacatcgtaggaaacttttatcttatgaacgtagatatttagctgctgatactacctcgttcaaatgcagcagggagggacacgagccacttttatctgtcaatggattgcaccagttacacatcctcgtattcttcttagcagtcattcatgtgctttacagtgctattacaatgatgcttggaagactaaagatacgtggatggaaggcatgggaagcggagacttcaactcataattatgagttcgccaatgctgcttcaagatttagacttactcatgaaacatcattcgtgagagcccattccagttttttgactaggattcccatcttcttctacattcgctgcttctttaggcagttctataggtctgtaaataagactgactacctcactttgcgcaatgggtttatcactgtccacctggctcctggaagtaaatttaatttccaaaagtatatcaaaagatcattagaagatgacttcaaggtggtcgtgggagttagtcctatcctctgggcatcagttgtagtttaccttctcatcaatgttaatggatggcacaccgtactttgggcagccttaattcctgttgttataattttggctgttggaacaaaacttcaagccatattggcaaatatggctcttgaaatcacggaaagacatgcagttgtccaaggaatgcctcttgtccaaggctcagacaaatacttttggtttggtcagccacagttagttctacatcttatccattttgctttgttccagaatgcgttccaaataacatatatcttgtggatatggtattcttttgggttgagaaactgtttccgtactgactacaagcttgcagtagtaaaagtagctctagggattttgatgctatgcctctgcagctatatcacccttccattatatgctcttgtaactcagatgggttcaaggatgaaaacagcaatatttgacgagcaaacaaacaaggctctgaagaaatggcacatggctgcgaagaagaagcagggaggagcagtgacgctaggaaagtcgagtgcacgaatcatggatggaagccccattggtaattcttcaacagtgcactccactggccccacactacaccgtttcaaaactactggccactcaacccgctcctcatcaacagcgtacgaggatcaagatcaagatcatgaatatgaatccgatggtgttgagttgtctccgttggcgtcgcaaacaacaagcttcattgtaagagttgatcatggcgaccaacaacaagcagaacatagacaagatagtgagggagaaaccaacagtagtagtgaaggtgaattctcatttgtcaaacctgaccctgtggaaattagaaccaccacatagcatatgatcatatattcatctctattcttatacataaatctttacataaaaaaaaaaaaaaa

SEQ ID NO: 9 - protein, 598 aa, Glycine max
MYSSKFRKLFCSVLFSWLCFGGLAMAAGESSSSSRDLDQTPTWAVAAVCTVFILVSIALEKSLHKVGTWLGQKKKKALLEALEKVKAELMILGFISLLLTFGQSYIVRICIPEKLADNMLPCPYKYKEDKKASDSEEEHRRKLLSYERRYLAADTTSFKCSREGHEPLLSVNGLHQLHILVFFLAVIHVLYSAITMMLGRLKIRGWKAWEAETSTHNYEFANAASRFRLTHETSFVRAHSSFLTRIPIFFYIRCFFRQFYRSVNKTDYLTLRNGFITVHLAPGSKFNFQKYIKRSLEDDFKVVVGVSPILWASVVVYLLINVNGWHTVLWAALIPVVIILAVGTKLQAILANMALEITERHAVVQGMPLVQGSDKYFWFGQPQLVLHLIHFALFQNAFQITYILWIWYSFGLRNCFRTDYKLAVVKVALGILMLCLCSYITLPLYALVTQMGSRMKTAIFDEQTNKALKKWHMAAKKKQGGAVTLGKSSARIMDGSPIGNSSTVHSTGPTLHRFKTTGHSTRSSSTAYEDQDQDHEYESDGVELSPLASQTTSFIVRVDHGDQQQAEHRQDSEGETNSSSEGEFSFVKPDPVEIRTTT

SEQ ID NO: 10 - DNA, 1179 bp, Glycine max
gctgttggaacaaaacttcaagccatactggcaaatatggctcttgaaatcacagaaagacatgcagttgtccaaggaatgcctcttgtccaaggctcagacaaatacttttggtttggtcagccacagttagttcttcatcttatccattttgctttgttccagaatgcgttccaaataacatatatcttgtggatatgggtaatgaagttcattacactatcttgactattggctaatgagttttgcttgtctatattcctgaataataaaagaacaattttttatttgcagtattcttttgggctgagaaattgtttccatgctgactacaagcttgcaatagtgaaagtagctttagggcttggggtgctatgcctctgcagctacatcacccttccattatatgctcttgttactcagatgggctcgagaatgaaaaaatcaatatttgacgaacaaacgtcgaaggcgttgaagaaatggcacatggctgtgaaaaagaagcaaggagtgaaacttggaaattccaaggtgcgagccctggatggaagcaccactgattcaacaatacactcttctggccccacactacaccgatacaaaactaccggtcactcaactcacttcacaccaaactatgatgaccaagatgattaccattctgacactgagttgtctcccatttcaccaacagcaaacttgatagtaagagtggaccatgatgaggaagaagcaaaagaaaatgaacacccaacagccaacaatcaagaggtgcctttacgtttgacaagcctggaaagaagcatgaaatagcataagaagttactctctattcaaccatttttggttcttttacttaatatatcttgaccttgtttagagctttctagatattgttatacttgataatgatatagtcagttttgtatattcttgttcttaaaacacttgccggcattggaaagtttcaccttgctcaaaatcatgcttactagctttgattcatgtgcttgagaaaaaggctcataaggagcaagtagaggacagaattgttgggtatattatgttatatgttcaaagtgtgttacataaatcccaattatataagctttctggtcaagtacaacaaatagaaaaatatacattcagttaaaaaaaaaaaaaaaaaaaaaaaaa

SEQ ID NO: 11 - protein, 240 aa, Glycine max
AVGTKLQAILANMALEITERHAVVQGMPLVQGSDKYFWFGQPQLVLHLIHFALFQNAFQITYILWIWYSFGLRNCFHADYKLAIVKVALGLGVLCLCSYITLPLYALVTQMGSRMKKSIFDEQTSKALKKWHMAVKKKQGVKLGNSKVRALDGSTTDSTIHSSGPTLHRYKTTGHSTHFTPNYDDQDDYHSDTELSPISPTANLIVRVDHDEEEAKENEHPTANNQEVPLRLTSLERSMK

SEQ ID NO: 12 - DNA, 813 bp, Oryza sativa
cctcgatgcaataactaatttaactataattgatttttcttgggttttctgcagggcaaggtggcgctgatgtcggcaaagagcatgcaccagctgcacattttcatcttcgtgctcgccgtgttccatgttacctactgcgtcatcaccatgggtttagggcgcctcaaagtgagtttgtcgttctgtccctcatgcacatgttttctctagttctagcaagattgtcagtccttcaaatggattgtttcgacaagaaacccaatttattaatttgccagtaaatatataataattggtctttcttggttttagatgaagaaatgggaagaagtgggagtcacaagaccaactcattggagtaccaagttcgcaatcggtagtgaattaagaatctccctaactatttcatttcagaacctttatgataatgtcttggaagaggaggagcaaatcagctgaaaatatgatcgatccatgcagatccttcacgattcaggttcacgcatcagacgtcgttcgtgaagcggcatctgggatcattctcaagcacccctgggctcagatggatcgtgagttatcaatctccgaatacatgcttgttttttattcttgcaactggcctagctgttccaattcaatccatattttttgaaaaaaaaaaatattcatgccgtgtttgttgttaggtagcattcttcaggcagttctttgggtccgtcaccaaggtggactacctgaccatgcggcaaggcttcatcaatgtatatactaatcaaacctgaccaattcaacattgatgatgcaaacaga

SEQ ID NO: 13 - DNA, 5277 bp, Oryza sativa
atggcaggtgggagatcgggatcgcgggagttgccggagacgccgacgtgggcggtggccgtcgtctgcgccgtcctcgtgctcgtctccgtcgccatggagcacggcctccacaacctcagccatgtacccgcgcgcgcacgcggtgtgctcatctctcgagttaatttggttgttgttgttgttgtgttcttgtgacatctcaattaacatccgatcctggtcgatcgatcgccctgtggtggcgctactgcttgcattgcagtggttccgtaggcggcagaagaaggccatgggcgacgccctcgacaagatcaaagcaggtcaccctcagcctcagctcaccctcagctagctcttactccctccatccctaaatgtttgacgccgttgacttttttaaatatgtttgaccgttcgtcttattcaaaaaatttaagtaattattaattctttttctaccatttgattcattgctaaatatactattatgtatacatatagttttacatatttcaccaaagtttttaaataagacgaatggtcaaacatgtttaaaacagtcaatggcatcaaacatttagggaagaagggaatattatattgctgctcccctctagccactttgctgcctccctcgtcattttttcaagtattttacgcaagactgggtcctccaaatcaaacgtcacaaataagccgtttatagtttcctttcgctttttaaggggggactacttgtatttaatcatggaggaaactaccagtcggatgtccgattacttaaaaaaaattcgggtgactaatttttttggttgatcatcggtgaaatattaggttatatatgttaaaaaaaatcagccacaaacaatgaaatattttgtgaaacacatattagacacgttgaaacgtatcattgttacgtataaaacatctaatgttaacagattaaaacatatgtttttctttcatcagaatataatcatgcgatatattattgtaaagatataattacaacgaatacaacggtgtgatcggattatatatatatattagtagtttaagagaaaaatcattttgaagattactagatacatacacgtatagatggatgaagtggagagagattagagataagtagttatatgaattttgtgaaacacacttaagacatatgttcaaacatactgctattatgtatgaaatattgagttttaacggtttaaaacacatattcttttaattagaatgtaataatgtgatatcttgttgtaaaatttaattacatctaatataacggtgtgattagattgtatgttggataacatgcccatcggttggcttattcagggaataagccaaacggtatatttgcaaacgaaaaataatttgtaaataaaacttttatgtatgtgttcttaacgatctagcagcaaaggctgaaaaataaacttcgatgaaaaatctcaaaatcaactcttaaaattcaaattttggcttataagtatagttcctaactagtttagaagaaaaaatatttaaagcggggaagaggaaaaggaataaactaatagctaaattattgcatgcatgtagcgatttgaggacgaccgagttgttttgtctggatcagccgaccgagacagagcaatcttctttaatcataaatgaccagaaaaaccataccagttcatcacaatggaccgagtcagagtcattacatatttttcattgttgcgcacaggattcaccatgttcttatgggaaatatttttaactctcaaatggttatgattttgaactctcatttttgagagagaattaacaagcgagcgagcaatcaggccaaaaaggagaaagaaaaattatttttgttaatttgtttttttaaggtagggtggaggagtcattacatgatttttttttatattccctcgttgattatatgctgttcaaatggttatgatttttttaaaaaataacaacaatacaaattagtatgtgatagatcatttcacgagcatgtaggattaaatttaacttctgtaaattacaaaacaaacaagtttaactgttaatatacattaaatttgttttttttcaacttaggaattgaattttatgtatatatttgtaaaatgatatattaatttatttttttaaaaaaataattatttagatggcacacaaactagaaaaccaccgcagttctcatatttcttgtcctatctgcacttgcagagctgatgctgctgggcttcatatccctgcttctcaccgtggcacaggcgcccatctccaagatctgcatccccaagtcggctgccaacatcttgttgccgtgcaaggcaggccaagatgccatcgaagaagaagcagcaagtgatcgccggtccttggccggcgccggcggcggggactactgctcgaaattcgatgtgagaataacaccagctgccggcaagcacaacctcgatgcaataactaatttaactataattgatttttcttgggttttctgcagggcaaggtggcgctgatgtcggcaaagagcatgcaccagctgcacattttcatcttcgtgctcgccgtgttccatgttacctactgcgtcatcaccatgggtttagggcgcctcaaagtgagtttgtcgttctgtccctcatgcacatgttttctctagttctagcaagattgtcagtccttcaaatggattgtttcgacaagaaacccaatttattaatttgccagtaaatatataataattggtctttcttggttttagatgaagaaatggaagaagtgggagtcacagaccaactcattggagtatcagttcgcaatcggtagtgaattaagaatctccctaactatttcatttcagaacctttatgataatgtcttgaaagaggaggagcaaatcagctgaaaaatatgatcgatccatgcagatccttcacgattcaggttcacgcatcagacgtcgttcgtgaagcggcatctgggatcattctcaagcacccctgggctcagatggatcgtgagttatcaatctccgaatacatgcttgttttttattcttgcaactggcctagctgttccaattcaatccatattttttgaaaaaaaaaatattcatgccgtgtttgttgttaggtagcattcttcaggcagttctttgggtccgtcaccaaggtggactacctgaccatgcggcaaggcttcatcaatgtatatactaatcaaacctgaccaattcaacattgatgatgcaaacagagaccaggtttttttttcgagtgtgcattgagtaatggttttagcttcttctcttttgcaggcgcatttgtcgcagaatagcaagttcgacttccacaaatacatcaagaggtctttggaggacgacttcaaagttgtcgttggcatcaggtccgtcctcgctttattaattataggactcttatattcaacattttttttataaagaaacatatttagtctccagttgtgtatgtgtatgtggatcttgacacatttggctggttttgcagcctccctctgtggttcgtcggaatccttgtactcttcctcgatatccacggtaatccttgtcctatttcattctgttttttactctcaaaaccttgttctgaattggtcttataatcaccatcgattttttttcaactttttccccgcgtgtaggtcttggcacacttatttggatctcttttgttcctctcatcgtaagagcgaaatttccctgtccaaagaaacagttaacataattaattatgctttaatttatcatgaaaattaatatgatcatataactaatgaacaaacattcatgtgaatgccaccgttgtctcagatcgtcttgttagttgggaccaagctagagatggtgatcatgcagatggcccaagagatacaggacagggccactgtgatccagggagcacctgtggttgaaccaagcaacaagtacttctggttcaaccgccctgactgggtcttgttcttcatacacctgacactcttccatgtacatgtttaaaacctaaaccttgctgctcaactacaaatagtactttatctttcacaattaacacctaattaactaacatagcatccatccatttgtggctactgatcgatgggacgacggatcgatgatcaccagaacgcatttcagatggcgcatttcgtatggactatggtgtgtatgctacttgcttagttgttgccattatcagttcttaagcaaattaagtgtgatgcatgcactgactaatgagacaaaaaatgacacagcttgttcatcgatctggttgttttgtgtgtgacaggcaacacctggtctgaagaaatgcttccatgaaaatatttggctgagcatcgtggaagtcattgtggggatctctcttcaggtgctatgcagctacatcaccttcccgctctacgcgctcgtcacacaggtgaacaagccattcacaaattctattagcagtttcttaattgacgacgctgttaatttttagacacacgttttgaccatttgtcttattaaaaatatttatgtaattatcatttgagttgttttatcactaaaagtactttttaaataatttatattttgcatttgtacaattcttttaataagataatggtcaaacaagtgtccaaaagttaacagcatcatctattaagaaaaggagggggtttttttttggaattttgcaaaatttgttcaaaatcagtccaaaaccttttttttgtttcgaaatttcagtttcactaccagtccccataaaatgtcttttctttatttccacaatattgaacccatgagatgccctttgtgttggtatgtgttttggccatcacttgcagatgggatcgaacatgaagaagacaatcttcgaggagcaaacgatgaaggcgctgatgaactggaggaagaaggcgatggagaagaagaaggtccgggacgcggacgcgttcctggcgcagatgagcgtcgacttcgcgacgccggcgtcgagccggtccgcgtcgccggtgcacctgctgcaggatcacagggcgaggtcggacgacccgccgagcccaatcacggtggcctcaccaccggcaccggaggaggacatatacccggtgccggcggcggctgcgtctcgccagctgctagacgacccgccggacaggaggtggatggcatcctcgtcggccgacatcgccgattctgatttttccttcagcgcacaacggtga

SEQ ID NO: 14 - protein, 540 aa, Oryza sativa
MAGGRSGSRELPETPTWAVAVVCAVLVLVSVAMEHGLHNLSHWFRRRQKKAMGDALDKIKAELMLLGFISLLLTVAQAPISKICIPKSAANILLPCKAGQDAIEEEAASDRRSLAGAGGGDYCSKFDGKVALMSAKSMHQLHIFIFVLAVFHVTYCVITMGLGRLKMKKWKKWESQTNSLEYQFAIDPSRFRFTHQTSFVKRHLGSFSSTPGLRWIVAFFRQFFGSVTKVDYLTMRQGFINVYTNQNSKFDFHKYIKRSLEDDFKVVVGISLPLWFVGILVLFLDIHGLGTLIWISFVPLIIVLLVGTKLEMVIMQMAQEIQDRATVIQGAPVVEPSNKYFWFNRPDWVLFFIHLTLFHNAFQMAHFVWTMATPGLKKCFHENIWLSIVEVIVGISLQVLCSYITFPLYALVTQMGSNMKKTIFEEQTMKALMNWRKKAMEKKKVRDADAFLAQMSVDFATPASSRSASPVHLLQDHRARSDDPPSPITVASPPAPEEDIYPVPAAAASRQLLDDPPDRRWMASSSADIADSDFSFSAQR

SEQ ID NO: 15 - DNA, 653 bp, Linum usitatissimum
acgaattcgagctcggtacccgatcatagcggcacatttagctcctggaagtgagagcagattcgacttccagaaatacgtcaacagatcacttgaggatgacttcaaagttgttgtagggatcagtccgattctatggttttttgcagtcttgttcctgctatccaatacacatggatgggtagcatatctgtggctaccctttattccactaatcataatcctggttgttggtacaaagctacaagtgatcataacccagctaggactaagcatacaagacagaggagatgtggtgaagggtgcccctgtggttcagccaggagatgacctcttctggtttggacgccctcgtctcgttctcttcctcatccatttctgcctcttccagaatgcatttcaacttgctttcttcatatggagtgtgtatgaattcggaatcaaaacttgcttccacgagaaaaccgaggacattgtcagagcttctggattggttccgaaccgtgatacatcagcaacacaaaccacggagctgtccaaaggcaagctgatgatggctgatacttgccttcctaccgaagatttggtagggatggtagtaacagctgctcattctgggaaaaggtttttcgtagattctatacgctatgatc

SEQ ID NO: 16 - protein, 217 aa, Linum usitatissimum
RIRARYPIIAAHLAPGSESRFDFQKYVNRSLEDDFKVVVGISPILWFFAVLFLLSNTHGWVAYLWLPFIPLIIILVVGTKLQVIITQLGLSIQDRGDVVKGAPVVQPGDDLFWFGRPRLVLFLIHFCLFQNAFQLAFFIWSVYEFGIKTCFHEKTEDIVRASGLVPNRDTSATQTTELSKGKLMMADTCLPTEDLVGMVVTAAHSGKRFFVDSIRYD

SEQ ID NO: 17 - DNA, 1239 bp, Triticum aestivum
atggcggacgacgacgagtaccccccagcgaggacgctgccggagacgccgtcctgggcggtggccctcgtcttcgccgtcatgatcatcgtgtccgtcctcctggagcacgcgctccataagctcggccattggttccacaagcggcacaagaacgcgctggcggaggcgctggagaagatcaaggcggagctcatgctggtgggcttcatctcgctgctgctcgccgtgacgcaggaccccatctccgggatatgcatctccgagaaggccgccagcatcatgcggccctgcaagctgccccctggctccgtcaagagcaagtacaaagactactactgcgccaaacagggcaaggtgtcgctcatgtccacgggcagcttgcaccagctgcacatattcatcttcgtgctcgccgtcttccatgtcacctacagcgtcatcatcatggctctaagccgtctcaaaatgagaacctggaagaaatgggagacagagaccgcctccctggaataccagttcgcaaatgatcctgcgcggttccgcttcacgcaccagacgtcgttcgtgaagcggcacctgggcctctccagcacccccggcgtcagatgggtggtggccttcttcaggcagttcttcaggtcggtcaccaaggtggactacctcaccttgagggcaggcttcatcaacgcgcatttgtcgcataacagcaagttcgacttccacaagtacatcaagaggtccatggaggacgacttcaaagtcgtcgttggcatcagcctcccgctgtggtgtgtggcgatcctcaccctcttccttgacattgacgggatcggcacgctcacctggatttctttcatccctctcgtcatcctcttgtgtgttggaaccaagctggagatgatcatcatggagatggccctggagatccaggaccgggcgagcgtcatcaagggggcgcccgtggttgagcccagcaacaagttcttctggttccaccgccccgactgggtcctcttcttcatacacctgacgctattccagaacgcgtttcagatggcacatttcgtgtggacagtggccacgcccggcttgaagaaatgcttccatatgcacatcgggctgagcatcatgaaggtcgtgctggggctggctcttcagttcctctgcagctatatcaccttcccgctctacgcgctcgtcacacagatgggatcaaacatgaagaggtccatcttc

SEQ ID NO: 18 - protein, 413 aa, Triticum aestivum
MADDDEYPPARTLPETPSWAVALVFAVMIIVSVLLEHALHKLGHWFHKRHKNALAEALEKIKAELMLVGFISLLLAVTQDPISGICISEKAASIMRPCKLPPGSVKSKYKDYYCAKQGKVSLMSTGSLHQLHIFIFVLAVFHVTYSVIIMALSRLKMRTWKKWETETASLEYQFANDPARFRFTHQTSFVKRHLGLSSTPGVRWVVAFFRQFFRSVTKVDYLTLRAGFINAHLSHNSKFDFHKYIKRSMEDDFKVVVGISLPLWCVAILTLFLDIDGIGTLTWISFIPLVILLCVGTKLEMIIMEMALEIQDRASVIKGAPVVEPSNKFFWFHRPDWVLFFIHLTLFQNAFQMAHFVWTVATPGLKKCFHMHIGLSIMKVVLGLALQFLCSYITFPLYALVTQMGSNMKRSIF

SEQ ID NO: 19 - DNA, 1934 bp, Arabidopsis thaliana
attccagtgttggtacataaaagactcttcctttgtctgttttttgttcccagattcatctttacttattgactaaattctctctggtgtgagaagtaaaatgggtcacggaggagaagggatgtcgcttgaattcactccgacgtgggtcgtcgccggagtttgtacggtcatcgtcgcgatttcactggcggtggagcgtttgcttcactatttcggtactgttcttaagaagaagaagcaaaaacccctttacgaagcccttcaaaaggttaaagaagagctgatgttgttagggtttatatcgctgttactgacggtattccaagggctcatttccaaattctgtgtgaaagaaaatgtgcttatgcatatgcttccatgttctctcgattcaagacgagaagctggggcaagtgaacataaaaacgttacagcaaaagaacattttcagacttttttacctattgttggaaccactaggcgtctacttgctgaacatgctgctgtgcaagttggttactgtagcgaaaagggtaaagtaccattgctttcgcttgaggcattgcaccatctacatattttcatcttcgtcctcgccatatcccatgtgacattctgtgtccttaccgtgatttttggaagcacaaggattcaccaatggaagaaatgggaggattcgatcgcagatgagaagtttgaccccgaaacagctctcaggaaaagaagggtcactcatgtacacaaccatgcttttattaaagagcattttcttggtattggcaaagattcagtcatcctcggatggacgcaatcctttctcaagcaattctatgattctgtgacgaaatcagattacgtgactttacgtcttggtttcattatgacacattgtaagggaaaccccaagcttaatttccacaagtatatgatgcgcgctctagaggatgatttcaaacaagttgttggtattagttggtatctttggatctttgtcgtcatctttttgctgctaaatgttaacggatggcacacatatttctggatagcatttattccctttgctttgcttcttgctgtgggaacaaagttggagcatgtgattgcacagttagctcatgaagttgcagagaaacatgtagccattgaaggagacttagtggtgaaaccctcagatgagcatttctggttcagcaaacctcaaattgttctctacttgatccattttatcctcttccagaatgcttttgagattgcgtttttcttttggatttgggttacatacggcttcgactcgtgcattatgggacaggtgagatacattgttccaagattggttatcggggtcttcattcaagtgctttgcagttacagtacactgcctctttacgccatcgtctcacagatgggaagtagcttcaagaaagctatattcgaggagaatgtgcaggttggtcttgttggttgggcacagaaagtgaaacaaaagagagacctaaaagctgcagctagtaatggagacgaaggaagctctcaggctggtcctggtcctgattctggttctggttctgctcctgctgctggtcctggtgcaggttttgcaggaattcagctcagcagagtaacaagaaacaacgcaggggacacaaacaatgagattacacctgatcataacaactgagcagagatattatcttttccatttagaggatcatcatcagattttagcttcaaggtccggttttgtggtttatacataagttatagtgacttgatttttttgttttgttacaaagttaccatctttggattagaattgggaaattgaatctgtttgtatattgtattatttggaacattgtggatgcccatggatatgtttctgttcaattattttggttttgggtaatgaaatttgaaaccaacgaaaaacc

SEQ ID NO: 20 - protein, 526 aa, Arabidopsis thaliana
MGHGGEGMSLEFTPTWVVAGVCTVIVAISLAVERLLHYFGTVLKKKKQKPLYEALQKVKEELMLLGFISLLLTVFQGLISKFCVKENVLMHMLPCSLDSRREAGASEHKNVTAKEHFQTFLPIVGTTRRLLAEHAAVQVGYCSEKGKVPLLSLEALHHLHIFIFVLAISHVTFCVLTVIFGSTRIHQWKKWEDSIADEKFDPETALRKRRVTHVHNHAFIKEHFLGIGKDSVILGWTQSFLKQFYDSVTKSDYVTLRLGFIMTHCKGNPKLNFHKYMMRALEDDFKQVVGISWYLWIFVVIFLLLNVNGWHTYFWIAFIPFALLLAVGTKLEHVIAQLAHEVAEKHVAIEGDLVVKPSDEHFWFSKPQIVLYLIHFILFQNAFEIAFFFWIWVTYGFDSCIMGQVRYIVPRLVIGVFIQVLCSYSTLPLYAIVSQMGSSFKKAIFEENVQVGLVGWAQKVKQKRDLKAAASNGDEGSSQAGPGPDSGSGSAPAAGPGAGFAGIQLSRVTRNNAGDTNNEITPDHNN

SEQ ID NO: 21 - DNA, 2148 bp, Arabidopsis thaliana
gaataacgccttctttagtttacagtctctcgttgcgggtcttatcttcttcttcttcttccctctttcgtctgctctatattatgaccagattcaaaattgcgtagcgtactaattcaatatagagagaagttagatcaaaagaagaacacgaaactctgactgtgtctctctttctcttcgtgtgtttctatatttatatataaaaagacaagatctctggtctggaattagaagaatcttatttggggttttttcttaggattaagctctaatggcagatcaagtaaaagagcggactttagaggagacctctacgtgggcagttgctgttgtttgctttgtcttactctttatttcgattgtcctcgaacattctattcacaaaattggaacctggtttaaaaagaagcacaagcaggctctttttgaagctcttgaaaaggtcaaagcagagcttatgctgttgggattcatatcactactactcacaattggacaaacaccaatctcaaatatctgcatctcccagaaagttgcgtcaacaatgcacccttgcagtgctgctgaagaagctaaaaaatacggcaagaaagacgccggaaagaaagatgatggagatggagataaacccggtcgaagacttcttcttgagttagctgaatcttatatccatagaagaagtttagccaccaaaggctatgacaaatgtgcagagaaggggaaagtggcttttgtatctgcttatggaatccaccagctgcatatattcatcttcgtgctcgcggttgttcatgttgtttactgcattgttacttatgctttcggaaagatcaagatgaggacgtggaagtcgtgggaggaagagacaaagacaatagagtatcagtattccaacgatcctgagaggttcaggtttgcgagggacacatcttttgggagaagacatctcaatttctggagcaagacgagagtcacactatggattgtttgtttttttagacagttctttggatctgtcaccaaagttgattacttagcactaagacatggtttcatcatggcgcattttgctcccggtaacgaatcaagattcgatttccgcaagtatattcagagatcattagagaaagacttcaaaaccgttgttgaaatcagtccggttatctggtttgtcgctgtgctattcctcttgaccaattcatatggattacgttcttacctctggttaccattcattccactagtcgtaattctaatagttggaacaaagcttgaagtcataataacaaaattgggtctaagaatccaagagaaaggtgatgtggtgagaggcgccccagtggttcagcctggtgatgacctcttctggtttggcaagccacgcttcattcttttccttattcacttggtcctttttacgaatgcatttcaacttgcattctttgcctggagtacgtatgaattcaatctcaataattgtttccatgaaagcactgcagatgtggtcattagacttgtagttggagctgttgtgcagatactttgcagctatgtgactcttccactctatgcacttgttactcagatgggtagtaaaatgaagccaacagtattcaacgatagagtagccacggcattaaagaagtggcatcacactgcaaagaacgagacgaaacacggaagacactcgggatccaacacacctttctctagccgtccaaccacaccaacacatggctcatctccaatccatctccttcacaatttcaataaccggagcgttgaaaattacccaagttctccttctcctagatactctggtcatggtcatcatgaacaccaattttgggatcctgagtctcaacaccaagaagctgaaacttccacacatcattctcttgcgcatgaaagctcagaacctgttcttgcatctgtggaacttcctcctataaggactagcaaaagcttaagagatttttcttttaagaaatgatgattcttgtttgctatatttgatttcgtacagtgggaaatttgtcatatgaaaataatttcttgtacattactagttggataagaaataaccatatctatatggatacatgatatgttattctctggcaatgcttaagagtttcacgaatt

SEQ ID NO: 22 - protein, 573 aa, Arabidopsis thaliana
MADQVKERTLEETSTWAVAVVCFVLLFISIVLEHSIHKIGTWFKKKHKQALFEALEKVKAELMLLGFISLLLTIGQTPISNICISQKVASTMHPCSAAEEAKKYGKKDAGKKDDGDGDKPGRRLLLELAESYIHRRSLATKGYDKCAEKGKVAFVSAYGIHQLHIFIFVLAVVHVVYCIVTYAFGKIKMRTWKSWEEETKTIEYQYSNDPERFRFARDTSFGRRHLNFWSKTRVTLWIVCFFRQFFGSVTKVDYLALRHGFIMAHFAPGNESRFDFRKYIQRSLEKDFKTVVEISPVIWFVAVLFLLTNSYGLRSYLWLPFIPLVVILIVGTKLEVIITKLGLRIQEKGDVVRGAPVVQPGDDLFWFGKPRFILFLIHLVLFTNAFQLAFFAWSTYEFNLNNCFHESTADVVIRLVVGAVVQILCSYVTLPLYALVTQMGSKMKPTVFNDRVATALKKWHHTAKNETKHGRHSGSNTPFSSRPTTPTHGSSPIHLLHNFNNRSVENYPSSPSPRYSGHGHHEHQFWDPESQHQEAETSTHHSLAHESSEPVLASVELPPIRTSKSLRDFSFKK

SEQ ID NO: 23 - DNA, 1587 bp, Arabidopsis thaliana
ttctcaactaaacaaatcagtcaccgggaaaaatataaagagatgacggataaagaagaaagcaaccactcctcggaggttggcgccgtccgatccctccaagaaactcccacttgggctctggccaccgtttgtttcttcttcatcgctgtttccatttgcctcgagcgtctcatcaatcttctatccactagacttaagaaaaatagaaaaacgtcgctacttgaagctgtagagaagctcaaatcagttctaatggtgctaggattcatgtcactaatgctaaatgtaactgaaggagaagtttcgaagatatgcattcctataaagtatgccaatcgaatgttgccatgccgtaaaaccataaaatcacataatgatgttagtgaagatgatgatgatgatgatggtgataatcatgacaacagcttctttcatcaatgctcttccaagggtaagacttcactaatatcagaagaagggttgactcagttgagttacttcttctttgtgttggcatgcatgcatattctctgcaatcttgctattcttctccttggaatggccaagatgagaaaatggaattcgtgggagaaagagacccaaacagtggagtatctagcagcaaacgatccgaataggtttagaataacaagagatacaacatttgctcgacgacacctgagttcatggactgaaacatcatttcaactttggattaaatgtttcttcaggcaattttacaactcagtggcgaaagtagattacctcacacttcggcatgggttcatctttgctcatgtatcgtcaaataatgctttcaacttccaaaactatatacagagatctttacatgaagattttaaaactgtggttggtataagtcctttaatgtggctaactgtggtcatctttatgctcctcgacgtttctggttggagagtatatttttatatgtcatttgtgccactcattatagttttagtgatcgggacgaaactagagatgatagtagcgaaaatggcggtcacaattaaggagaataacagtgtaattagaggaacccctctcgttgagtcaaacgacacgcatttctggttttccaatcctcgttttctcttaagcattctacactacacgctgtttctgaacaccttcgagatggcatttattgtgtggatcacttggcaatttgggattaactcttgctaccatgataaccaagggatcataatcacacgacttgtattagcggtgacagtccagttcttgagcagctacattacattgcctctttatgccattgtaactcagatggggtcaagctacaagagagcaatcttagaagaacaactagcaaatgtgttaaggcactggcaagggatggttagagacaagaaaaagactatccaaacgccagacaccgacaacaacagtaacaataacaatggtgatattgattctggagaaagtccggttcagacagaagttgcttctgagtttagattttcgggtagacaatcgccgattttacaagagatacagatacaagagaaaactgaaaggtgatcagattgatggaaaatg

SEQ ID NO: 24 - protein, 508 aa, Arabidopsis thaliana
MTDKEESNHSSEVGAVRSLQETPTWALATVCFFFIAVSICLERLINLLSTRLKKNRKTSLLEAVEKLKSVLMVLGFMSLMLNVTEGEVSKICIPIKYANRMLPCRKTIKSHNDVSEDDDDDDGDNHDNSFFHQCSSKGKTSLISEEGLTQLSYFFFVLACMHILCNLAILLLGMAKMRKWNSWEKETQTVEYLAANDPNRFRITRDTTFARRHLSSWTETSFQLWIKCFFRQFYNSVAKVDYLTLRHGFIFAHVSSNNAFNFQNYIQRSLHEDFKTVVGISPLMWLTVVIFMLLDVSGWRVYFYMSFVPLIIVLVIGTKLEMIVAKMAVTIKENNSVIRGTPLVESNDTHFWFSNPRFLLSILHYTLFLNTFEMAFIVWITWQFGINSCYHDNQGIIITRLVLAVTVQFLSSYITLPLYAIVTQMGSSYKRAILEEQLANVLRHWQGMVRDKKKTIQTPDTDNNSNNNNGDIDSGESPVQTEVASEFRFSGRQSPILQEIQIQEKTER

SEQ ID NO: 25 - DNA, 1971 bp, Arabidopsis thaliana
gctaaactttcttaatcacagatgcagattgattctccatttttctcgggagaaaagttcctcgccggagatggagcatatgatgaaagaaggaaggtctcttgcagagacgccgacttactctgttgcttcggttgttactgttttggtctttgtttgctttctcgttgaacgcgccatttacagatttggaaagtggttaaagaagactagaagaaaggcactttttacttcacttgagaaaatgaaagaggagttgatgttgctgggacttatatcacttctgttgtcacaaagcgcgagatggatttcagaaatctgtgttaactcttcccttttcaatagtaaattctacatttgctctgaagaggactatggaatccataagaaagttcttctggaacacacctcttctacaaaccagagctccttacctcatcatggaatacatgaagcctctcatcaatgtggtcatggccgtgaaccatttgtgtcgtatgagggactcgagcaactcctaagattcttattcgtcctgggtatcactcatgttctatacagtggcattgccattggtttagccatgagcaagatttacagttggagaaaatgggaagcccaagcgatcataatggctgaatcagatatccacgcaaagaagaccaaggtgatgaagcgtcagtctacatttgttttccatcatgcctctcatccttggagtaacaatcgttttctcatttggatgctttgtttcctgcggcaatttagaggctccatacgaaagtctgactacttcgcacttcggttaggtttcctcactaaacataatttgccatttacatacaacttccatatgtatatggtacggacgatggaagatgagtttcatggcattgttggaattagctggccactttgggtttacgctatagtatgcatctgcataaatgttcatggcctgaatatgtacttttggatatcattcgttcctgccattcttgtcatgttggttggaaccaaacttgagcatgttgtctccaagcttgctctcgaggttaaggagcagcagacaggcacatctaatggggctcaagtcaaaccacgtgatgggctcttctggtttgggaaaccagaaattctgctacggttgatacaatttatcatttttcagaatgcatttgaaatggcaacattcatctggttcttgtggggaatcaaggaaagatcttgcttcatgaagaaccatgtgatgatatcaagccggctaatttctggggttctcgttcagttctggtgtagttatggcactgtgcctctcaatgtaatcgttactcagatgggatctcggcataagaaagctgtgatagcagagagcgtaagagactcacttcacagttggtgcaagagagtgaaagagaggtctaagcacacgagatcagtgtgttcccttgacacagcaacaatagacgagagagacgagatgacagtggggacattgtctaggagctcatcgatgacttcactgaatcagattaccataaactccatagaccaagcagagtctatattcggagcagcagcttcatccagcagtcctcaagatggatacacgtcgagggtggaagaatatctgtctgaaacatacaataacatcggttcgataccgcctttaaacgatgagattgagattgagattgaaggtgaagaagataatggagggagaggaagtgggagtgatgagaataacggtgatgctggagaaacacttcttgagttgtttaggaggacttgatcttgtttctgcttttagaagcttggttcagttgtaattacttgacatccatacaagaataacaattaacaaagtaaatcttttgttggtccagtaaaaaaaagtattcaatgatcaaagtacatcttttgttggttcagaggtaaatttaaaatataatagcatggattggaacgaga

SEQ ID NO: 26 - protein, 573 aa, Arabidopsis thaliana
MEHMMKEGRSLAETPTYSVASVVTVLVFVCFLVERAIYRFGKWLKKTRRKALFTSLEKMKEELMLLGLISLLLSQSARWISEICVNSSLFNSKFYICSEEDYGIHKKVLLEHTSSTNQSSLPHHGIHEASHQCGHGREPFVSYEGLEQLLRFLFVLGITHVLYSGIAIGLAMSKIYSWRKWEAQAIIMAESDIHAKKTKVMKRQSTFVFHHASHPWSNNRFLIWMLCFLRQFRGSIRKSDYFALRLGFLTKHNLPFTYNFHMYMVRTMEDEFHGIVGISWPLWVYAIVCICINVHGLNMYFWISFVPAILVMLVGTKLEHVVSKLALEVKEQQTGTSNGAQVKPRDGLFWFGKPEILLRLIQFIIFQNAFEMATFIWFLWGIKERSCFMKNHVMISSRLISGVLVQFWCSYGTVPLNVIVTQMGSRHKKAVIAESVRDSLHSWCKRVKERSKHTRSVCSLDTATIDERDEMTVGTLSRSSSMTSLNQITINSIDQAESIFGAAASSSSPQDGYTSRVEEYLSETYNNIGSIPPLNDEIEIEIEGEEDNGGRGSGSDENNGDAGETLLELFRRT

SEQ ID NO: 27 - DNA, 1666 bp, Arabidopsis thaliana
ttaccgccgtttcttggccgtagaagatataaatattgatagtcgtatataaaaaaaaaacatataagacaaggtacataagaaaaaaacaatttcgttaaagaaaaaatggctggaggaggaggtggtagcacttctggagaaggtcctagagagctcgatcagacaccgacatgggccgtctccactgtttgtggcgttatcatcttgatctctatcgttctagagctcatgattcacaaaatcggagaggttttcactgaaagaaggaagaaagctttgtacgaagcgcttcaaaagatcaagaacgagcttatggttttgggattcatttctttgttattaacatttggacaaaactacatagcaagtttgtgtgtggcgtcaagatacggccatgcgatgtccttttgtggtccatacgatggtccaagtggtgagtctaagaagccaaagactaccgaacacttagaacgtcgtgttttggctgatgctgctccagctcagtgcaagaagggctatgtaccgcttatatcactcaacgcgttgcatcaagtgcatatcttcatctttttcttggctgtgtttcatgtcatttatagtgctattaccatgatgcttggacgagcaaagattcgtggatggaaagtttgggaggaagaagtcataaatgatcatgaaatgatgaatgacccttcaaggtttaggctcacacatgagacatcatttgttagagagcatgtcaatccttgggccaaaaatagattctcattctacgttatgtgtttctttcgtcaaatgctgagatctgtcagaaaatctgattatttgacaatgcgtcatggtttcataagtgtccatttggcaccgggtatgaagtttaatttccaaaaatacatcaaaagatcattggaagacgacttcaaggtagtcgtgggaataagtcccgagctatgggcctttgtaatgctctttttgctctttgatgttcacgggtggtatgttactgctgtgatcaccatgattcctccacttttgacattagcgataggaaccaagcttcaagccatcatctcagacatggcgttggaaattcaggagagacacgccgtgatacaagggatgccacttgtcaatgtctctgatcgacatttctggtttagtcgtcccgccttagtcctccatatcatccacttcattctcttccagaatgcttttgagatcacctacttcttctggatatggtatgagttcgggttacggtcctgttttcatcaccatttcgcgctaataatcataagggttgctcttggggtgggagtacaatttctctgcagttacatcacacttcctctttacgctctcgtgactcagatgggatcaacgatgaagcgatcggtgtttgatgatcaaacgtcaaaggcattaaagaattggcataaaaatgcaaagaaaaagagcgaaactcctggtcaaacgcagcctccattgcctaatctccgacctaaaaccggcggcgatattgagtctgcttctccggccaatatcacggctagtgttgatgttaaagaaagtgatcaatctcaatctagagacctcttaagcggtccctaaaaaaccaaacgggtcaagacattactttcacttaaccggcttactctaagtc

SEQ ID NO: 28 - protein, 501 aa, Arabidopsis thaliana
MAGGGGGSTSGEGPRELDQTPTWAVSTVCGVIILISIVLELMIHKIGEVFTERRKKALYEALQKIKNELMVLGFISLLLTFGQNYIASLCVASRYGHAMSFCGPYDGPSGESKKPKTTEHLERRVLADAAPAQCKKGYVPLISLNALHQVHIFIFFLAVFHVIYSAITMMLGRAKIRGWKVWEEEVINDHEMMNDPSRFRLTHETSFVREHVNPWAKNRFSFYVMCFFRQMLRSVRKSDYLTMRHGFISVHLAPGMKFNFQKYIKRSLEDDFKVVVGISPELWAFVMLFLLFDVHGWYVTAVITMIPPLLTLAIGTKLQAIISDMALEIQERHAVIQGMPLVNVSDRHFWFSRPALVLHIIHFILFQNAFEITYFFWIWYEFGLRSCFHHHFALIIIRVALGVGVQFLCSYITLPLYALVTQMGSTMKRSVFDDQTSKALKNWHKNAKKKSETPGQTQPPLPNLRPKTGGDIESASPANITASVDVKESDQSQSRDLLSGP

SEQ ID NO: 29 - DNA, 1942 bp, Arabidopsis thaliana
ctgtgagttctaatggcggatcaagttaaagaaaagacattggaggaaacttctacatgggccgtcgcggtggtttgcttcgtcttgcttctgatttcgatcgttattgaaaaacttattcataaaattggatcctggtttaaaaagaagaacaaaaaagctctatatgaagcacttgaaaaagtgaaagcagagcttatgctgatgggattcatatcactacttctaacaattggacaaggatatatctcaaatatttgcatccctaagaacatcgcagcatcgatgcacccttgtagtgcatccgaagaagcaagaaagtatggtaagaaagatgtcccaaaggaagatgaagaagaaaacttgcgtcgaaagcttctacagttagttgattctcttattcctcgaaggagtttggctactaaaggttatgataagtgtgcagagaagggaaaagtcgcttttgtatcggcttatggcatgcatcagctgcatatattcatctttgttcttgcggtttgtcatgtgatctactgcattgttacttatgctttgggaaagaccaaaatgagaaggtggaagaagtgggaagaggagactaagactatcgaatatcagtattcacacgatcccgagaggtttaggtttgcgcgggatacatctttcgggcgtagacatctgagtttctggagcaaatcaactattacgctgtggattgtatgtttcttcaggcagttctttagatctgtaaccaaagttgattacttaacactgagacatggtttcatcatggcccatttggctcctgggagcgacgcaaggttcgatttccgaaagtatattcagagatcactagaggaagacttcaaaaccattgtcgagatcaaccctgtgatctggttcatagctgtgctattcctcctgacaaacacaaacggattgaattcttacctctggctaccgttcattcccttcatcgtgattcttattgttggaacaaaacttcaagtgataataacaaaactaggacttagaatccaagagaaaggcgacgtagtgaaaggcacaccgctagttcaacccggtgatcatttcttctggttcggtcgtccacgtttcattctcttcctcattcacttagtcctcttcacgaacgcgtttcaactagctttctttgtctggagtacgtatgaattcggtctaaagaactgtttccatgaaagcagagttgacgtgatcatcagaatctcaatcggacttttagttcagattctttgcagctacgttactcttcctctatatgctcttgttactcaaatgggttcaaagatgaaaccaacagtgttcaacgagagagtagcaacagcgttaaagagttggcatcacacagctaagaaaaatatcaaacatggaagaacttcagaatcaacaacacctttctctagtagaccaacaacaccaactcatggttcttctccgattcatctccttcgtaatgctcctcacaaacgaagcagaagcgttgacgaaagctttgcgaattcgttttctccgagaaactctgatttcgattcgtgggatcctgagtctcaacatgaaactgctgagacttcgaattcgaatcatcgttctaggtttggagaagaagaatcggagaaaaagtttgtttcttcatcagtggaacttcctcctggacctggacaaatacgaacacagcatgagattagtactataagcttaagggatttttcgtttaagcgatgatttaatgttttcttaattcattatttagtgatttgttaatatgctaaatgaaaccagttcttgtaaattttctatactatgtttgttaaaaccagcccttgaagctttaatttctcaatgtaaaagtctgatgattttggattttaagactatggagattcatgggtagttggtgaag

SEQ ID NO: 30 - protein, 583 aa, Arabidopsis thaliana
MADQVKEKTLEETSTWAVAVVCFVLLLISIVIEKLIHKIGSWFKKKNKKALYEALEKVKAELMLMGFISLLLTIGQGYISNICIPKNIAASMHPCSASEEARKYGKKDVPKEDEEENLRRKLLQLVDSLIPRRSLATKGYDKCAEKGKVAFVSAYGMHQLHIFIFVLAVCHVIYCIVTYALGKTKMRRWKKWEEETKTIEYQYSHDPERFRFARDTSFGRRHLSFWSKSTITLWIVCFFRQFFRSVTKVDYLTLRHGFIMAHLAPGSDARFDFRKYIQRSLEEDFKTIVEINPVIWFIAVLFLLTNTNGLNSYLWLPFIPFIVILIVGTKLQVIITKLGLRIQEKGDVVKGTPLVQPGDHFFWFGRPRFILFLIHLVLFTNAFQLAFFVWSTYEFGLKNCFHESRVDVIIRISIGLLVQILCSYVTLPLYALVTQMGSKMKPTVFNERVATALKSWHHTAKKNIKHGRTSESTTPFSSRPTTPTHGSSPIHLLRNAPHKRSRSVDESFANSFSPRNSDFDSWDPESQHETAETSNSNHRSRFGEEESEKKFVSSSVELPPGPGQIRTQHEISTISLRDFSFKR

SEQ ID NO: 31 - DNA, 1695 bp, Arabidopsis thaliana
atgatcacaagaagcaggtgtcgaagatctttgttatggtttctagtgttccatggcggagctacagccaccggagctccctctggtgggaaagagctttctcagacgcctacttgggcagtcgccgtcgtctgcacctttctcatcctcatttcccatctccttgaaaagggtcttcaaagactcgccaactggctatggaagaagcataaaaactctctccttgaagccttagagaaaatcaaagctgagctgatgatccttggattcatttctttattactcacttttggagaaccatatattctcaagatctgtgttcctcgaaaagctgctctctctatgttaccttgtttatctgaagacacagtgcttttccagaaacttgctccatcatctcttagcaggcatcttttggctgctggtgatacatctattaattgcaaacaaggatctgagccactcataacattgaaaggcttgcaccaacttcacatcttgttgttcttcttggccatctttcatatcgtatatagtttaatcaccatgatgcttagcaggctcaagattcgtggatggaaaaagtgggagcaagagacattatctaatgactatgagttttctattgatcattcaagacttaggctcactcatgagacttcttttgtgagagaacatacaagtttctggacaacaactcctttcttcttttacgtcggatgcttctttaggcagttctttgtatctgttgaaagaaccgactacttgactctgcgccatggattcatctctgcccatttagctccaggaagaaagttcaacttccagagatatatcaaaagatctctcgaggatgatttcaagttggtagttggaataagtccagttctttgggcatcatttgtaatcttcttgctgttcaatgttaatggctggagaacattgttttgggcatcgatacctcctctactcataatcctagctgttggaacaaagcttcaagcaattatggcaacaatggcgctagaaatcgtagagacacatgcagtagttcaggggatgcctttagtgcaaggttcagatcgatacttttggttcgactgtcctcaactacttcttcatcttatccactttgccttgtttcagaatgctttccagataacacacttcttctggatatggtattcttttggattaaaatcatgcttccataaagatttcaatcttgtagtcagcaaactctttctatgcctaggagctttgatcttatgcagctacatcactctcccattgtacgccctcgttactcagatgggttcacacatgaagaaagcagtgtttgatgagcaaatggcaaaggcattgaagaagtggcacaaagacatcaaattgaagaaaggtaaagcgaggaagctcccgagcaagacacttggtgtttcagagagtttcagcctctcttcctcttcctctgcaaccactcttcaccgttccaagaccactggtcactcttctaacatcatatactacaaacaagaagatgaagaagacgaaatgtctgatcttgaagctggagcagaggatgctattgacaggattcaacaacaggagatgcaattccacaactcttagctgctttagtttcttctgttgttacttcatagttcaaatgaaacgatgctgtttactaacttagtc

SEQ ID NO: 32 - protein, 542 aa, Arabidopsis thaliana
MITRSRCRRSLLWFLVFHGGATATGAPSGGKELSQTPTWAVAVVCTFLILISHLLEKGLQRLANWLWKKHKNSLLEALEKIKAELMILGFISLLLTFGEPYILKICVPRKAALSMLPCLSEDTVLFQKLAPSSLSRHLLAAGDTSINCKQGSEPLITLKGLHQLHILLFFLAIFHIVYSLITMMLSRLKIRGWKKWEQETLSNDYEFSIDHSRLRLTHETSFVREHTSFWTTTPFFFYVGCFFRQFFVSVERTDYLTLRHGFISAHLAPGRKFNFQRYIKRSLEDDFKLVVGISPVLWASFVIFLLFNVNGWRTLFWASIPPLLIILAVGTKLQAIMATMALEIVETHAVVQGMPLVQGSDRYFWFDCPQLLLHLIHFALFQNAFQITHFFWIWYSFGLKSCFHKDFNLVVSKLFLCLGALILCSYITLPLYALVTQMGSHMKKAVFDEQMAKALKKWHKDIKLKKGKARKLPSKTLGVSESFSLSSSSSATTLHRSKTTGHSSNIIYYKQEDEEDEMSDLEAGAEDAIDRIQQQEMQFHNS

SEQ ID NO: 33 - DNA, 2368 bp, Arabidopsis thaliana
aataaataaagagtttagtcattttatcgtaaaaccaaaagaaagagaaaaagattaatcatcatcaatctaaagatttaggaatctacaatttcccagtagacagatttcacttttgcgtcttcttcttattccatttgatctaagtttctcgaaataggtacaaagttcgagtctttttcttgtaaccgcgtgaaaccaccaagaaacctatagtaagagtttccgcaaatgtgaattaaagacagctcatcactctgtcaaacttctacagtattaagttaacgaagcaggtacagccaaactaaaataacgcggagttatgggaatcatcgacggttctttgcttcggcggttgatttgtctctgtctctggtgtcttctcggtggaggagtgacggtggttacggcggaggatgagaagaaagtggtacataaacagcttaatcaaactccgacttgggctgttgctgctgtttgtactttcttcatcgttgtttctgttcttcttgaaaaacttcttcacaaagttggaaaggttctatgggatcggcacaagacagctcttcttgacgctttggagaagatcaaagcagagctgatggttcttggattcatctctttgcttctgacatttggacaaacctacattttggatatttgtatcccttcacatgttgctcgtacgatgctcccgtgtcctgctcctaacttgaaaaaggaggatgatgacaatggtgaaagtcacaggagactcttgtcgtttgagcacagatttttatctggaggtgaagcatctcccactaaatgcacgaaggagggttatgtagagcttatctctgccgaggcactccatcagttgcacatccttatattcttcttagccattttccacgttctttacagcttcttaactatgatgcttggaaggttgaagattcgcggatggaagcattgggagaatgagacatcatcccataattacgagttttcaacagacacttccagattcaggctaactcatgaaacatcttttgtgagagcgcacaccagtttctggacccggattccattctttttctatgttggatgctttttcagacagtttttcagatccgttgggagaactgactatttgacattgagaaatggtttcatcgctgttcatttagctccaggaagtcaatttaacttccaaaaatacattaaaagatcgttggaggatgatttcaaggtagtcgttggagtcagccctgtcttgtggggatcttttgtgctattcctcctcctaaatattgacggcttcaagatgatgttcatcggcactgcaataccggttattatcattttagctgtagggacaaagcttcaagcgattatgacaaggatggctcttggtatcacagatagacatgcggtagttcaaggaatgccgcttgtacaaggcaacgatgagtatttctggttcggtcgtccccatttgattctccatctcatgcatttcgccttgtttcagaacgcatttcagatcacttatttcttctggatatggtattcctttggatcagattcttgctaccatcctaatttcaagattgcacttgtaaaagtagcgattgctttaggagtattgtgtctttgcagctacatcacacttcctctttacgcactcgtaactcagatgggttctcggatgaaaaaatcggtattcgatgaacaaacgtcaaaagcactcaagaaatggagaatggcagtgaagaagaagaaaggtgtgaaagccactactaagagactaggtggagatggaagtgcgagccctacggcatcgacagttaggtctacttcgtctgtacgttcattgcagcgttacaaaaccacaccacattcgatgagatacgaaggacttgaccctgaaacatcggatctcgacacagataatgaagctttgactcctcccaaatctcctccaagcttcgagcttgttgtgaaagttgaaccaaataagaccaataccggtgagactagccgtgacactgaaactgattctaaagagttctctttcgtcaagcctgctccgagtaatgaatcatctcaagaccggtgagactagtcaggattgtgaaggtgtagcctgcgccaagtaaagacttcttttcgagttaagcctgcgccaagtaaagaaccatcgtagtaaagatgtgtcactccagagtttttttttttttggtttcatttatctaatgctattgtaaatcaatgccgtggcgataaagtaatgtattgatgaccatattaattaaaataggctgaagaaaatgagggacaaatattaagaatcagtttagtaaaaaggttaaaagactcgtgt

SEQ ID NO: 34 - protein, 593 aa, Arabidopsis thaliana
MGIIDGSLLRRLICLCLWCLLGGGVTVVTAEDEKKVVHKQLNQTPTWAVAAVCTFFIVVSVLLEKLLHKVGKVLWDRHKTALLDALEKIKAELMVLGFISLLLTFGQTYILDICIPSHVARTMLPCPAPNLKKEDDDNGESHRRLLSFEHRFLSGGEASPTKCTKEGYVELISAEALHQLHILIFFLAIFHVLYSFLTMMLGRLKIRGWKHWENETSSHNYEFSTDTSRFRLTHETSFVRAHTSFWTRIPFFFYVGCFFRQFFRSVGRTDYLTLRNGFIAVHLAPGSQFNFQKYIKRSLEDDFKVVVGVSPVLWGSFVLFLLLNIDGFKMMFIGTAIPVIIILAVGTKLQAIMTRMALGITDRHAVVQGMPLVQGNDEYFWFGRPHLILHLMHFALFQNAFQITYFFWIWYSFGSDSCYHPNFKIALVKVAIALGVLCLCSYITLPLYALVTQMGSRMKKSVFDEQTSKALKKWRMAVKKKKGVKATTKRLGGDGSASPTASTVRSTSSVRSLQRYKTTPHSMRYEGLDPETSDLDTDNEALTPPKSPPSFELVVKVEPNKTNTGETSRDTETDSKEFSFVKPAPSNESSQDR

SEQ ID NO: 35 - DNA, 1096 bp, Arabidopsis thaliana
caatgcgtattctctactctgttcaacgtcgttctttggccgacgctcctccagtaaattgcaagaaagattatgtcgcacttatatcattaaacgcattacatcaagtgcatatattcatattcttcttggccgtgttccatgttatatatagtgctataaccatgatgcttggaagagccaagattcgtggctggaaagtatgggagcaagaggtcatccatgaacaagaaatgatgaatgatccatcaagatttagactcacacatgagacatcatttgtccgagaacatgtcaattcttgggctagcaataaattcttcttctacgttatgtgcttcttccgtcaaatacttagatcagtgaggaagtctgattacttgacaatgcgacatggattcataagtgttcatttggcacctggtatgaagtttgattttcaaaagtacatcaaaagatctttggaagacgacttcaaggtggttgtaggaataagacccgagctttgggcctttgtaatgttgtttttactcttcgatgttcacggatggtatgttactgccgtaatcaccatgattcctcctctattaacactagccataggaacaaagctacaagccattatatcgtacatggcattggagattcaagagagacatgcagtaattcaagggatgccagttgtcaacgtctcagaccaacatttttggtttgaaaaacccgatctagtacttcatatgatccacttcgttctgtttcagaatgcttttgagataacttattttttttggatatggtatgagtttgggctaaggtcctgttttcatcaccattttggccttataatcattcgtgtctgcctaggggtgggagtacaattcctatgcagttatatcacattgcccctttacgctctcgtcactcagatgggatccacaatgaagcgatcagtgtttgatgagcaaacttcaaaagcattagaacaatggcataagaaggcgaggaaaaagaatgaaaagtgagaataattggcttctccttttaatctatggatagaccaaatttaacagtctaatagtttggttacttaatagtc

SEQ ID NO: 36 - protein, 460 aa, Arabidopsis thaliana
MAGGGGGGGGEGPRQLDQTPTWAVSTVCGVIILISIILELIIHKVGEVFERKKKKALFEALEKIKNELMVLGFISLLLTFGQNYIASICVPSRYGHAMSFCGPYDGPSEDDRKKLKKTDHAMRILYSVQRRSLADAPPVNCKKDYVALISLNALHQVHIFIFFLAVFHVIYSAITMMLGRAKIRGWKVWEQEVIHEQEMMNDPSRFRLTHETSFVREHVNSWASNKFFFYVMCFFRQILRSVRKSDYLTMRHGFISVHLAPGMKFDFQKYIKRSLEDDFKVVVGIRPELWAFVMLFLLFDVHGWYVTAVITMIPPLLTLAIGTKLQAIISYMALEIQERHAVIQGMPVVNVSDQHFWFEKPDLVLHMIHFVLFQNAFEITYFFWIWYEFGLRSCFHHHFGLIIIRVCLGVGVQFLCSYITLPLYALVTQMGSTMKRSVFDEQTSKALEQWHKKARKKNEK

SEQ ID NO: 37 - DNA, 1836 bp, Arabidopsis thaliana
cacgtacgtaatcaaggaccaagggattttcttcttttggctaccatggccacaagatgcttttggtgttggaccactttgctcttctgctctcagctgcttaccggctttgcccgagcttcctctgcaggcggcgccaaagagaaaggactctcccaaactcccacctgggccgttgccctcgtctgtaccttcttcattcttgtctccgtcctcctcgagaaggctcttcacagagttgccacgtggttgtgggagaaacataagaactctctgcttgaagccttggagaaaataaaggcggagctgatgattctaggattcatttccttgttgctcaccttcggagagcagtacattctcaagatttgtattcctgaaaaggctgcagcctctatgttaccttgtccagctccttctactcatgaccaagacaagacccaccgcagacgtctagctgctgctacgacctcttcccgctgcgatgagggtcatgaaccactcatacctgccacgggtttgcaccagctacacattctattgttcttcatggctgcctttcatatcctctacagtttcatcaccatgatgcttggcagactcaagatccgtggctggaaaaagtgggagcaggagacatgttctcatgattacgagttttcaatcgatccatcaagattcagactcactcatgagacgtcctttgttagacaacattccagtttctggacaaaaatccccttcttcttttatgctgggtgcttcctacagcagtttttccgatctgtagggaggactgactacttaactctgcgccatggcttcatcgctgcccatttagctccaggaagaaagttcgacttccagaagtatatcaaaagatcattggaagacgatttcaaggtggtagttggaataagtcctcttttgtgggcatcatttgtaattttcctacttctgaatgttaatggctgggaagcattgttttgggcgtcaatcctacctgtacttatcattctagctgtcagtacgaagcttcaagcgatcctaacaagaatggctctgggaatcacggagagacacgcagttgttcaagggatacctctcgtgcatggttcagataagtacttttggtttaatcgccctcagttgctacttcatcttcttcacttcgccttatttcagaatgctttccagctaacatacttcttctgggtctggtattcctttgggctaaaatcttgctttcacacggatttcaaactagtcatcgtaaaactctctctaggcgttggagctttgattttgtgcagctacatcacacttcctttgtatgcactagttactcagatgggttcaaacatgaagaaagctgtgtttgatgagcaaatggcaaaagcgttgaagaaatggcacatgactgtgaagaagaagaaaggcaaagcgagaaagccaccaacagagacccttggtgtttctgacactgtcagcacctctacctcatcctttcacgcctctggagccactctactccgctccaagaccactggtcactcgacagcctcttatatgagtaatttcgaggaccaaagcatgtctgatcttgaagctgagccattatcccctgaaccaatagaggggcacactctcgtcagggttggtgatcagaacacagagatagaatatactggagatattagtcctggaaaccaattctcctttgtgaagaacgttcctgctaatgatattgactaatattcaaaatgaatgcagaacaaatccatcatccggtctttattttctattacatgtatgccaacaattgcttcgccaagt

SEQ ID NO: 38 - protein, 569 aa, Arabidopsis thaliana
MATRCFWCWTTLLFCSQLLTGFARASSAGGAKEKGLSQTPTWAVALVCTFFILVSVLLEKALHRVATWLWEKHKNSLLEALEKIKAELMILGFISLLLTFGEQYILKICIPEKAAASMLPCPAPSTHDQDKTHRRRLAAATTSSRCDEGHEPLIPATGLHQLHILLFFMAAFHILYSFITMMLGRLKIRGWKKWEQETCSHDYEFSIDPSRFRLTHETSFVRQHSSFWTKIPFFFYAGCFLQQFFRSVGRTDYLTLRHGFIAAHLAPGRKFDFQKYIKRSLEDDFKVVVGISPLLWASFVIFLLLNVNGWEALFWASILPVLIILAVSTKLQAILTRMALGITERHAVVQGIPLVHGSDKYFWFNRPQLLLHLLHFALFQNAFQLTYFFWVWYSFGLKSCFHTDFKLVIVKLSLGVGALILCSYITLPLYALVTQMGSNMKKAVFDEQMAKALKKWHMTVKKKKGKARKPPTETLGVSDTVSTSTSSFHASGATLLRSKTTGHSTASYMSNFEDQSMSDLEAEPLSPEPIEGHTLVRVGDQNTEIEYTGDISPGNQFSFVKNVPANDID

SEQ ID NO: 39 - DNA, 2361 bp, Arabidopsis thaliana
aaaaataaaaatacgaaaaaacaagtatttttcactttgctcaagaccatctctgcacatgaagttatgaactaagtgagaccagaagaacttctttcgatcgatttctacagtgattctgtttgctcaagactacaactttgtgtactttctctttcaacgagctgtgtatcgtgtacttcatggtgtgttcctctgcttctgattgagattgaatctcctgtggtttttcagttgctccggtgatcggctccggtggattgaaatttcaaagcttttcctaatttcttggagtctacagatttcaaagtttctgggttactaccaaagtagaatctcagtttttgtctaagctaatcagatttctgcgattctgattgttaactaatccaagaacgatcgatgggggtttaaagttgaatttgggtctagaggaggaacttgttttgtttttgtgaagaatcagactttaaaagaaatgggagaaggagaagaaaatggaaatgaagcagattcaaatgagagatctttggcattgtcacctacttggtctgttgctattgtcttgactgttttcgttgttgtttctttgatcgttgagcgctccatttatcgtctcagcacttggttgagaaaaacaaagaggaaacctatgtttgctgcattagagaagatgaaagaagagttgatgctgcttggcttcatttcacttctattaacagctacctcaagtacaatagccaatatatgtgtcccttcaagtttctacaatgacagatttcttccgtgtacacgatctgagatccaagaggagctcgaaagcggttcaactgtcaagcggaatcttttaacaaaatccctctttttcaacatctttaggagaaggttggatgtcataaaacgaaccacctgcagcgaggggcatgagccgtttgtttcatatgaaggccttgaacagttgcatcgcttcatctttataatggcagttactcatgtaacttatagctgcttgaccatgctccttgcaatcgtcaagattcatagttggaggatatgggaagatgtagctcgtctagatagacacgattgcttaactgcggtggcacgtgaaaaaatattccgaaggcaaacaacgtttgtccagtatcatacatcagcacctctggccaagaatagaattcttatatgggtgacatgtttcttccggcaattcggacgttctgttgatcgttctgactatcttactctccgcaaaggattcattgtgaatcaccacctcacattaaagtacgattttcacagctacatgattcgttctatggaagaagagttccaaaggatcgttggtgtgagtgggccgctttggggtttcgtagttgctttcatgctctttaacataaaaggatcaaatctttacttctggatagcgattattcctgtaacccttgttcttctggttggtgctaagttgcaacacgtaatagcaactttggcattggagaatgccgggttaaccgagtatccttccggagtaaagctgagacctcgagatgaacttttctggttcaataaaccagaactactcttgtcccttatccatttcattctgtttcagaattcttttgaactggcttcgttcttctggttctggtggcagtttgggtacagctcttgcttccttaagaaccattaccttgtttacttcagacttcttttagggtttgctggacagtttctatgcagctacagtacactgccactatatgcactagtcactcagatgggaacgaactataaagcggctcttatacctcagagaataagagagacgattcgcggttggggtaaagcaacgagaaggaaaagaaggcacgggttatatggcgatgattcaacagttagaacagaaacaagcacaattgcatcacttgaagaatatgatcatcaagtgcttgacgttactgaaacttctttcgaacaacaacggaaacaacaagaacaaggtactactgagcttgagttgcaaccaatccaacctcgtaatgattgtgtacccaatgatacttcaagtagagtcggaacacctcttcttcgaccttggctctccatttcttcaccaacaacgaccatagagttgagatcagaacctatggaaacactctcgagatcgtcttccttgccaagtgagaagagagtctgaatataaataagttcgtaggagtaacagtagaaaacaagtatgtctcgatgaacatagattgaaaacttacatcaatcaagtgttagcaacactaatatttgtatgtgctctacaaaaaaattatattgcaatattgagaaacatttaaatttgttagaacc

SEQ ID NO: 40 - protein, 573 aa, Arabidopsis thaliana
MGEGEENGNEADSNERSLALSPTWSVAIVLTVFVVVSLIVERSIYRLSTWLRKTKRKPMFAALEKMKEELMLLGFISLLLTATSSTIANICVPSSFYNDRFLPCTRSEIQEELESGSTVKRNLLTKSLFFNIFRRRLDVIKRTTCSEGHEPFVSYEGLEQLHRFIFIMAVTHVTYSCLTMLLAIVKIHSWRIWEDVARLDRHDCLTAVAREKIFRRQTTFVQYHTSAPLAKNRILIWVTCFFRQFGRSVDRSDYLTLRKGFIVNHHLTLKYDFHSYMIRSMEEEFQRIVGVSGPLWGFVVAFMLFNIKGSNLYFWIAIIPVTLVLLVGAKLQHVIATLALENAGLTEYPSGVKLRPRDELFWFNKPELLLSLIHFILFQNSFELASFFWFWWQFGYSSCFLKNHYLVYFRLLLGFAGQFLCSYSTLPLYALVTQMGTNYKAALIPQRIRETIRGWGKATRRKRRHGLYGDDSTVRTETSTIASLEEYDHQVLDVTETSFEQQRKQQEQGTTELELQPIQPRNDCVPNDTSSRVGTPLLRPWLSISSPTTTIELRSEPMETLSRSSSLPSEKRV

SEQ ID NO: 41 - DNA, 1843 bp, Arabidopsis thaliana
ttgagcagtgaaattaatggcaataaaagagcgatcactagaggaaacaccaacatgggctgttgctgtagtttgcttcgttctccttttcatttccatcatgatcgaatacttcttgcactttattggtcactggtttaagaagaagcacaagaaagctttatctgaagctcttgaaaaggttaaagcagaactgatgcttctgggattcatatcgcttctattggttgtattgcaaacaccagtctccgagatttgcattccaagaaatattgctgcgacttggcatccttgtagcaaccatcaagaaatcgccaaatatggtaaagattatatcgacgatggccgcaaaattcttgaagattttgactccaacgacttttacagtcctcgccgaaatttagccaccaagggttatgacaaatgcgcagaaaaggggaaagtagcattagtatctgcctatggtatccaccagctgcatatattcatctttgtgctcgctgtttttcatgttctctactgcattataacctatgctttaggaaagaccaagatgaagaaatggaagtcatgggagagagagaccaaaacaattgagtaccaatatgccaatgatccagagaggttcagatttgcaagagatacatcgttcggacgtagacatctgaatatatggagcaagtctacctttaccctctggattacatgtttcttcagacagttctttggatcagtgacaaaagtagattatcttacactaagacatggctttattatggcacatttgccagcaggaagtgccgctcgtttcgatttccaaaaatacattgaaagatctttggaacaagatttcacggtggttgtcggtataagcccactgatatggtgcattgctgtcttattcatattgaccaatacacatggatgggactcatatctttggctgcccttccttcccttgattgtgatattgatagtaggagcaaaacttcaaatgataatatcgaaattaggattaaggattcaagaaaaaggagatgtggttaaaggagctcctgtggttgaaccgggcgatgatctcttttggtttggtcgtcctcgtttcattctcttcctcattcacttggttcttttcacgaatgcatttcaactggctttcttcgtttggagcacttacgaattcacactcaaaaactgcttccaccacaaaacagaagacattgcaattaggatcaccatgggggtattaatacaagttctatgcagctacatcactctacctctctatgctcttgttactcagatgggaacatcaatgaggccgacaatattcaacgacagggtagccaatgcattgaagaaatggcaccacacagccaagaaacagacgaaacatggacactcaggatctaacacacctcactcgagccgtcctactacgccaacacatggcatgtcaccggtgcatctcctccacaactacaataaccgcagcctcgaccaacaaaccagcttcactgcttctccttctcctcctagattctctgattatagcggccaaggccatggccatcagcatttcttcgaccctgaatctcagaatcactcttaccaacgtgagatcacagattctgaattcagcaatagtcatcatccccaagttgacatggcaagtcctgttagagaagagaaggagattgttgagcatgtcaaggttgatttgtctgagtttacgttcaagaagtgataggactatactttttacattctgtttgttcattctttgtaaatgatatgaacttaggaatgaacacttttcttatttctttatctgaattttgct

SEQ ID NO: 42 - protein, 576 aa, Arabidopsis thaliana
MAIKERSLEETPTWAVAVVCFVLLFISIMIEYFLHFIGHWFKKKHKKALSEALEKVKAELMLLGFISLLLVVLQTPVSEICIPRNIAATWHPCSNHQEIAKYGKDYIDDGRKILEDFDSNDFYSPRRNLATKGYDKCAEKGKVALVSAYGIHQLHIFIFVLAVFHVLYCIITYALGKTKMKKWKSWERETKTIEYQYANDPERFRFARDTSFGRRHLNIWSKSTFTLWITCFFRQFFGSVTKVDYLTLRHGFIMAHLPAGSAARFDFQKYIERSLEQDFTVVVGISPLIWCIAVLFILTNTHGWDSYLWLPFLPLIVILIVGAKLQMIISKLGLRIQEKGDVVKGAPVVEPGDDLFWFGRPRFILFLIHLVLFTNAFQLAFFVWSTYEFTLKNCFHHKTEDIAIRITMGVLIQVLCSYITLPLYALVTQMGTSMRPTIFNDRVANALKKWHHTAKKQTKHGHSGSNTPHSSRPTTPTHGMSPVHLLHNYNNRSLDQQTSFTASPSPPRFSDYSGQGHGHQHFFDPESQNHSYQREITDSEFSNSHHPQVDMASPVREEKEIVEHVKVDLSEFTFKK

SEQ ID NO: 43 - DNA, 1699 bp, Arabidopsis thaliana
gtttattctctgccttaaataataataatattaattcagagttgtctccatcttggaggtgcagaaatggcagaagcaaggtctggttctcttgagtatacacccacatgggtcgttgcgtttatctgtttcatcattgttctcttatctcttctcgctgaacgtggtcttcatcatcttggaaagtgtctgaagcgtaggcaacaagatgccttgttcgaagccttgcagaaactcaaagaagaattgatgcttcttggatttatctccctgatgttaacggtatctcaggccgcaattcggcatatctgtgtcccaccagctcttgtaaacaacatgtttccctgtaagaagccattggaggagcatcatgcgcctaaatcatctcattcgattatcaacaatgcacgacatctcctttcaacaggagaaagtccagaccattgtgctgccaaggggcaggttccattagtatctgtggaagcgttgcatcaactccatatcttcatctttgtgctagcggtttttcacgtcatcttctgcgcctcaaccatggttcttggaggagcaagaatacaacaatggaaacattgggaggattggttcaagaaacgtccttctcaaaagggcactacaaggcgtggtcatcatgctcatgctcacgagttattcagtgcaaatcacgagttcttcgagatgcatgctggaggattttggagaagatctgttgtcatcagctgggtgagatcattcttcaagcagttttatggttctgtcaccaaatcagaatacatagctctgcgacaagcattcatcatgagtcattgccgcacaaacccatcatttgattttcacaagtacatgctaagaacactggaaatagatttcaagaaagttgtgagcataagttggtatctatggctctttgtcgtcgtctttttgctgctcaatgttggaggatggaacacttacttctggttatctttcttgcctttgattttgttactaatggtgggtgccaagttggaatatataataagtagcttagctttggatgtttccgagaagcgaagccgcgcggaagaagcagtgatcacaccttctgatgaactcttttggttccataggccaggcattgttctccaactcatccatttcattctctttcagaattcatttgagattgctttcttcttctggattttgttcacatacggaatacattcatgtatcatggagaaactaggctaccttatcccaagactcgtcatgggagtgttagtccaagtgctttgcagttacagcacattaccactatatgcccttgttacacaaatgggtagcaaattcaagaaagggatattcgacaatgtagtacagtccacactggaaggatggttagaagatacgaggaacagaggcgaatccacgagcgaggctcataggatagagatgcaacctacaactcctgaatcttataatgtccaaagtgaaaacccttaattaagttgcaatttgtgtcgtaatccaataaggctatccctttattatttcacatttagagctctcatcagaggattgtgtgcatctatgacattgacgccaacgttcctttctacttttgaggctactagattaagttgacttgttactttttataagaaagcttcgatgatacatggattgatatataattaaa

SEQ ID NO: 44 - protein, 478 aa, Arabidopsis thaliana
MAEARSGSLEYTPTWVVAFICFIIVLLSLLAERGLHHLGKCLKRRQQDALFEALQKLKEELMLLGFISLMLTVSQAAIRHICVPPALVNNMFPCKKPLEEHHAPKSSHSIINNARHLLSTGESPDHCAAKGQVPLVSVEALHQLHIFIFVLAVFHVIFCASTMVLGGARIQQWKHWEDWFKKRPSQKGTTRRGHHAHAHELFSANHEFFEMHAGGFWRRSVVISWVRSFFKQFYGSVTKSEYIALRQAFIMSHCRTNPSFDFHKYMLRTLEIDFKKVVSISWYLWLFVVVFLLLNVGGWNTYFWLSFLPLILLLMVGAKLEYIISSLALDVSEKRSRAEEAVITPSDELFWFHRPGIVLQLIHFILFQNSFEIAFFFWILFTYGIHSCIMEKLGYLIPRLVMGVLVQVLCSYSTLPLYALVTQMGSKFKKGIFDNVVQSTLEGWLEDTRNRGESTSEAHRIEMQPTTPESYNVQSENP

SEQ ID NO: 45 - DNA, 1733 bp, Arabidopsis thaliana
ttggtttcttgcacgcgaagaaagaaacatctgaaaatttgttcataaagaattcggttgaagaaatgagagaagaaacagaaccaagcgagagaacgttgggtttaacaccaacttggtcagttgctacagtattgactatctttgtatttgtttctttgatcgttgaacgttccattcatcggcttagtaactggttgcaaaagactaagagaaaacctttgtttgctgcattggagaaaatgaaagaagagctgatgctgctcggattcatatcgcttcttcttacagctacctctagcacaatagctaacatatgcgtctcttcaagtttccacaacgatagatttgtcccatgtacgccttctgagattaatgaggaacttgaaagtactatttctactgtcaagcggactcagttaacgagatctctcttcttgcacactctgaggagaagattgagtggtataggagaggatacgtgcagcgaggggcatgagccatttctttcatatgaaggcatggaacaattgcatcgcttcatatttataatggcagtcactcatgtaacttacagctgcttgacaatgcttcttgcaatcgtgaagattcatagatggaggatatgggaagacgaggttcatatggatcgaaatgattgcttaactgtggttgcacgcgaaaagattttccgaaggcaaacaacatttgtccagtatcatacatctgctcctctggtcaagaatagattacttatatgggtgatatgtttcttcagacagtttggacattctgttgttcgttctgactatcttacactccgaaagggattcatcatgaaccatcacttaacattgacatacgattttcatagctatatgatccgctctatggaagaagagttccaaaagattgttggcgtcagtgggccactttggggttttgtagttggtttcatgcttttcaacataaaaggatcaaatctttatttctggctagcgatcattccaatcactcttgttcttttggttggtgcaaagttgcaacatgtcatcgcaactttagcattggagaacgctagtataacagaatatgcttctggtataaagctgagacctcgcgatgaacttttttggttcaagaaaccggaattactcttatcccttatccacttcattcagtttcagaatgcttttgaactggcttctttcttctggttttggtggcaatttggatacaattcttgcttcctaaggaaccatttacttgtctacttgcgacttattctagggttttctggacaatttttatgtagctacagcacactgccactctatgcacttgtcactcagatgggaacaaactacaaggcagcattgttacctcaaagggtaagagagacaattaatggttggggaaaagcaacaagacggaaaagaagacatggtttatatggagatgattcaactattcgaacagaaacaagcacaattgcatctgttgatgaatacaatgaccaggtgcttgatgtttccgaaacctctccagttcaagataacgagcttgagcttcagcttattcgcggagcttgtggaaattctagtagtgttgaaacaccgatcttgcggccttgtgcttcaatatcttcaacgactttctcgaggctacagacagaaacaacagactcactctcaaggtcatcctcattgccaatgagaagagaatgttaagat

SEQ ID NO: 46 - protein, 554 aa, Arabidopsis thaliana
MREETEPSERTLGLTPTWSVATVLTIFVFVSLIVERSIHRLSNWLQKTKRKPLFAALEKMKEELMLLGFISLLLTATSSTIANICVSSSFHNDRFVPCTPSEINEELESTISTVKRTQLTRSLFLHTLRRRLSGIGEDTCSEGHEPFLSYEGMEQLHRFIFIMAVTHVTYSCLTMLLAIVKIHRWRIWEDEVHMDRNDCLTVVAREKIFRRQTTFVQYHTSAPLVKNRLLIWVICFFRQFGHSVVRSDYLTLRKGFIMNHHLTLTYDFHSYMIRSMEEEFQKIVGVSGPLWGFVVGFMLFNIKGSNLYFWLAIIPITLVLLVGAKLQHVIATLALENASITEYASGIKLRPRDELFWFKKPELLLSLIHFIQFQNAFELASFFWFWWQFGYNSCFLRNHLLVYLRLILGFSGQFLCSYSTLPLYALVTQMGTNYKAALLPQRVRETINGWGKATRRKRRHGLYGDDSTIRTETSTIASVDEYNDQVLDVSETSPVQDNELELQLIRGACGNSSSVETPILRPCASISSTTFSRLQTETTDSLSRSSSLPMRREC

SEQ ID NO: 47 - DNA, 1639 bp, Arabidopsis thaliana
ccggagcaaaaaaatggcgggaggaggaacgaccttagagtacacaccaacttgggtggttgctctcgtctgctccgtcattgtctctatctcttttgccgtcgagcgtctcattcaccgtgccggaaagcactttaagaacaacgatcagaagcagctttttggggcattacaaaagatcaaagaagaactcatgctagtagggttcatatcgttgctattatcggtcggccagtctaaaatcgcaaagatttgcatatcaaaggagttaagtgaaaagtttctcccttgtacgaaacctgcaggggctgagaagtcccttaaagactcctcccatttccagttcagcttcaccggccgtcatctcctcgccggagatgcccccgccggtgactactgctccctaaagggaaaagtaccaataatgtcattatcagctctgcacgagcttcatatattcatcttcgtattagctgttgcccacattattttctgcctcttaaccattgtttttggtaccatgaagataaagcaatggaaaaaatgggaggataaggttttagagaaggacttcgacacagaccaaatgatccagaagaaattcacacacgttcaagaacacgaattcatcaggtcacgatttcttggggttggaaaagctgatgcttccttgggatgggtgcaatcgtttatgaaacagtttcttgcgtcagtcaatgaatcagattacatcacaatgaggctaggctttgtcacgactcattgcaagaccaacccaaaattcaatttccataagtatttaatgcgtgcccttaattctgatttcaagaaagtcgtcggtatcagttggtacctttgggtatttgtggttctcttcttgcttctaaacattgttgcatggcatgtttacttctggctagcttttattcctttgatccttttacttgccgtggggacgaagttagagcatatcatcacagatttggctcatgaagttgcagagaaacacattgctgtggaaggagatttggttgtccgaccatcagatgatttgttctggtttcaaagtccccggctagttctcttcttgatccatttcattcttttccaaaactccttcgaaatcgcctacttcttctttatcctttttcaatttggttgggattcatgcatcatggatcatgtcaagttcgtaattccaagactcgtcatcggggtaataattcagcttctctgcagttacagtaccttaccgctctatgcactcgtaactcagatgggaagttcattcaaaggtgcaatattcaacgagcagacacaggaacatcttgttggatgggcaaaaatggctaaaaggggagtgaagaaaggcgcaacacaagtaggtaccagtcatgatgctactagtccaagaccgtctattcagttgaactccttgctaggaaaaggctcctctcaacaaaatcagaaccccaaggaaaaatcagagattgctcaccatgattaacaaacaaaaacttgttctgacattttttttgttgttgtgatttttgattttttgaacttgttcgactttgtttggtatatataactgattgaaaggcgttgtaacaaataaacacagcaaagatgctgaatc

SEQ ID NO: 48 - protein, 497 aa, Arabidopsis thaliana
MAGGGTTLEYTPTWVVALVCSVIVSISFAVERLIHRAGKHFKNNDQKQLFGALQKIKEELMLVGFISLLLSVGQSKIAKICISKELSEKFLPCTKPAGAEKSLKDSSHFQFSFTGRHLLAGDAPAGDYCSLKGKVPIMSLSALHELHIFIFVLAVAHIIFCLLTIVFGTMKIKQWKKWEDKVLEKDFDTDQMIQKKFTHVQEHEFIRSRFLGVGKADASLGWVQSFMKQFLASVNESDYITMRLGFVTTHCKTNPKFNFHKYLMRALNSDFKKVVGISWYLWVFVVLFLLLNIVAWHVYFWLAFIPLILLLAVGTKLEHIITDLAHEVAEKHIAVEGDLVVRPSDDLFWFQSPRLVLFLIHFILFQNSFEIAYFFFILFQFGWDSCIMDHVKFVIPRLVIGVIIQLLCSYSTLPLYALVTQMGSSFKGAIFNEQTQEHLVGWAKMAKRGVKKGATQVGTSHDATSPRPSIQLNSLLGKGSSQQNQNPKEKSEIAHHD